(19) United States
(12) Patent Application Publication
(10) Pub. No.: US 2025/0375520 A1
(43) Pub. Date: Dec. 11, 2025

(54) PRODUCTION OF IMMUNE CELLS

(71) Applicant: ADAPTIMMUNE LIMITED, Abingdon, Oxfordshire (GB)

(72) Inventors: Garth HAMILTON, Abingdon, Oxfordshire (GB); Nail FATKHUTDINOV, Abingdon, Oxfordshire (GB)

(21) Appl. No.: 18/872,766

(22) PCT Filed: Jun. 12, 2023
(86) PCT No.: PCT/EP2023/065689

§ 371 Date: Dec. 6, 2024

(30) Foreign Application Priority Data
Jun. 10, 2022 (GB) 2208545.0

(51) Int. Cl. A61K 40/11; A61K 35/17; A61K 40/32; A61K 40/42; C07K 14/725; C12N 5/074; C12N 5/0783; C12N 5/10; C12N 9/22; C12N 15/113; C12N 15/864

(52) U.S. Cl. CPC A61K 40/11; A61K 35/17; A61K 40/32; A61K 40/42; C07K 14/7051; C12N 5/0636; C12N 5/0696; C12N 5/10; C12N 9/226; C12N 15/113; C12N 15/8645; C12N 2310/20; C12N 2506/11; C12N 2510/00; C12N 2750/14143

(57) ABSTRACT

This invention relates to methods for producing immune cells expressing a therapeutic antigen receptor. An immune cell is provided that comprises a heterologous expression cassette. The heterologous expression cassette comprises (a) a coding sequence for a production TCR, (b) a constitutive promoter operably linked to the coding sequence, (c) a 5′ targeting site, and optionally (d) a 3′ targeting site. An expression construct is then introduced into the immune cell at the site of the heterologous expression cassette. The expression construct comprises a coding sequence for a therapeutic antigen receptor and the therapeutic antigen receptor is then expressed in the immune cell. Methods, reagents for use in the methods and immune cells produced by the methods are provided.

### FIELD

This invention relates to the production of immune cells, for example for use in immunotherapy.

### BACKGROUND

Immunotherapeutics are poised to transform the cancer treatment landscape with the promise of long-term survival (McDermott et al., Cancer Treat Rev. 2014 October; 40 (9): 1056-64). There is a clear unmet medical need for new immunomodulatory drugs to expand the scope of patient population eligibility and range of tumor types. In addition, new agents are needed to enhance the magnitude and duration of anti-tumor responses. The development of these agents has been possible because of the in-depth understanding of the basic principles controlling T-cell immunity over the last two decades (Sharma and Allison, Cell. 2015 Apr. 9; 161 (2): 205-14). This typically requires tumor specific immune cells, such as CD4+ and CD8+ T-cells, recognising tumor-associated peptide antigens presented by MHC molecules. Different vaccination strategies and adoptive transfer of ex-vivo expanded tumor infiltrated lymphocytes have in some cases demonstrated the ability of tumor specific immune cells to treat late-stage cancer (Rosenberg et al., Nat Med. 2004 September; 10 (9): 909-15).

However, current adoptive immune cell therapies are limited by a lack of suitable patient and tumor-specific immune cells and there is a need for therapeutically sufficient and functional antigen-specific immune cells for effective use in immunotherapy.

### SUMMARY

The present inventors have developed methods that involve generating immune cells that comprise a heterologous expression cassette for a “placeholder” production T-cell receptor (TCR). After production, the immune cells may then be primed for therapeutic use in a patient by replacing the heterologous expression cassette with an expression construct encoding a therapeutic antigen receptor, for example an antigen receptor that binds to cancer cells in the patient. These methods may be useful, for example, in the production of immune cells, such as allogeneic immune cells, for use in immunotherapy, in particular the production of “personalised” immune cells with a therapeutic antigen receptor that is selected to bind to the cancer cells of a patient.

A first aspect of the invention provides a method for producing an immune cell expressing a therapeutic antigen receptor comprising;
(i) providing an immune cell comprising a heterologous expression cassette,
wherein the heterologous expression cassette comprises;
   (a) a coding sequence for a production T cell receptor (TCR),
   (b) a constitutive promoter operably linked to the coding sequence,
   (c) a targeting site, and
(ii) introducing an expression construct comprising a coding sequence for a therapeutic antigen receptor into the immune cell at the site of the heterologous expression cassette, such that the therapeutic antigen receptor is expressed in the immune cell.

In some embodiments of the first aspect of the invention, the heterologous expression cassette may be replaced by the expression construct comprising the coding sequence for the therapeutic antigen receptor. For example, a method for producing an immune cell expressing a therapeutic antigen receptor may comprise;
(i) providing an immune cell comprising a heterologous expression cassette,
wherein the heterologous expression cassette comprises;
   (a) a coding sequence for a production T cell receptor (TCR),
   (b) a constitutive promoter operably linked to the coding sequence, and
   (c) a targeting site, and
(ii) replacing the heterologous expression cassette in the immune cell with an expression construct comprising a coding sequence for a therapeutic antigen receptor,
such that the therapeutic antigen receptor is expressed in the immune cell.

A second aspect of the invention provides a method for producing an immune cell expressing a therapeutic antigen receptor comprising;
(i) providing an induced pluripotent stem cell (iPSC) comprising a heterologous expression cassette at a site in the genome of the iPSC,
wherein the heterologous expression cassette comprises;
   (a) a coding sequence for a production T cell receptor (TCR),
   (b) a constitutive promoter operably linked to the coding sequence, and
   (c) a targeting site,
(ii) differentiating the iPSC into an immune cell, and
(iii) introducing an expression construct comprising a coding sequence for a therapeutic antigen receptor into the immune cell at the site of the heterologous expression cassette,
such that the therapeutic antigen is expressed in the immune cell.

For example, a method for producing an immune cell expressing a therapeutic antigen receptor may comprise;
(i) providing an induced pluripotent stem cell (iPSC) comprising a heterologous expression cassette at a site in the genome of the iPSC,
wherein the heterologous expression cassette comprises;
   (a) a coding sequence for a production T cell receptor (TCR),
   (b) a constitutive promoter operably linked to the coding sequence, and
   (c) a targeting site,
(ii) differentiating the iPSC into an immune cell, and
(iii) replacing the heterologous expression cassette in the immune cell with an expression construct comprising a coding sequence for a therapeutic antigen receptor,
such that the coding sequence for the therapeutic antigen is expressed in the immune cell,
thereby producing an immune cell expressing the therapeutic antigen receptor.

The iPSC may be provided in methods of the second aspect by transfecting an IPSC with a nucleic acid comprising the heterologous expression cassette, such that the heterologous expression cassette is integrated into the genome of the IPSC.

A third aspect of the invention provides an immune cell comprising a heterologous expression cassette integrated into the genome thereof,
wherein the heterologous expression cassette comprises;
   (a) a coding sequence for a production T cell receptor (TCR),
   (b) a constitutive promoter operably linked to the coding sequence, and
   (c) a targeting site.

In some embodiments of the first to the third aspects, the therapeutic antigen receptor may specifically bind to cancer cells.

A fourth aspect of the invention provides an IPSC comprising a heterologous expression cassette integrated into the genome thereof,
wherein the heterologous expression cassette comprises;
   (a) a coding sequence for a production T cell receptor (TCR),
   (b) a constitutive promoter operably linked to the coding sequence, and
   (c) a targeting site.

In some embodiments, of the first to the fourth aspects, the targeting site may be a 5′ targeting site. The heterologous expression cassette may further comprise a 3′ targeting site.

The heterologous expression cassette of the first to the fourth aspects may further comprise a coding sequence for a poly (A) sequence.

A fifth aspect of the invention provides a population of immune cells produced by a method of the first or second aspect.

A sixth aspect of the invention provides a pharmaceutical composition comprising a population of immune cells of the fifth aspect and a pharmaceutically acceptable excipient.

A seventh aspect of the invention provides a method of treatment comprising administering a therapeutically effective dose of a population of immune cells of the fifth aspect to an individual in need thereof.

The individual may have a cancer condition.

Other aspects and embodiments of the invention are described in more detail below.

### BRIEF DESCRIPTION OF THE FIGURES

FIG. 1 shows a schematic view of an example of a six-stage method for generating T cells from iPSCs.

Placeholder A2M10 to A2M4 exchange was performed in 15F2_AAVS1^{-/A2M10LP} and 16D5_AAVS1^{A2M10LP/A2M10LP} early-stage iT cell progenitors on stage 4. Placeholder exchange on late stage iT cell progenitors was performed on stage 6 following activation with CD3/28.

FIG. 2 shows a schematic overview of an example of a TCR “landing pad”. A ‘placeholder’ TCR is integrated within the genome using CRISPR+AAV. This expression cassette, under control of a constitutive promoter, drives the TCR expression required to facilitate full iT-cell differentiation. The candidate therapeutic TCR replaces the ‘placeholder’ TCR during a final edit step in iT-cells. CRISPR/Cas9 targeting of TRAC leads to two (2) simultaneous events: (i) Excision of ‘placeholder’ TCR and replacement with functional candidate TCR that will be under control of an endogenous promoter and (ii) knockout of the natural endogenous TRAC locus.

FIG. 3 describes a targeting strategy for the insertion of TCR “landing pad” into the last exon of a coding gene. The targeting vector contains left and right homology arms (HA) corresponding to the 300-1000 nucleotides of genomic DNA 5′ and 3′ of the CRISPR guide RNA, truncated TRAC domain containing the 5′ landing pad excision sequence, poly A signal (PA), an exogenous promoter, 2A “like” skip sequences, the “placeholder” TCR and an additional PA sequence. The TRAC domain “placeholder” TCR contains the landing pad 3′ excision site. Following monoallelic integration into the desired genomic location, a second targeting vector is designed to be integrated following the excision of the landing pad using guide RNA's targeting the excision sites. The secondary targeting vector contains left and right homology arms (HA), 2A “like” skip sequences and the therapeutic TCR. The TCRα chain of the therapeutic TCR contains a truncated TRAC domain. The full-length TRAC domain is reconstituted when the therapeutic TCR is knocked into the landing pad.

FIG. 4 describes an alternative targeting strategy for the insertion of TCR “landing pad” into the last exon of a coding gene. In this scenario the 5′ and 3′ excision sequences correspond to region of B2M (Chr 15 44715435 to 44715475) containing a guide RNA recognition sequence. The excision sequence flanks the placeholder TCR. The flanking regions can be modified to contain any guide sequences of interest. The targeting vector contains left and right homology arms (HA) corresponding to the 300-1000 nucleotides of genomic DNA 5′ and 3′ from the integration site, poly A signal (PA), an exogenous promoter, 2A “like” skip sequences, the “placeholder” TCR and an additional PA sequence. Following monoallelic integration into the desired genomic location, a second targeting vector is designed to be integrated following the excision of the landing pad using guide RNA's targeting the excision sites. The secondary targeting vector contains left and right homology arms (HA), 2A “like” skip sequences and the therapeutic TCR.

FIG. 5 describes a targeting strategy for the insertion of TCR “landing pad” into a genomic “safe-harbour” locus. The targeting vector contains left and right homology arms (HA) corresponding to the 300-1000 nucleotides of genomic DNA 5′ and 3′ from the integration site, the truncated TRAC domain containing the 5′ landing pad excision sequence, poly A signal (PA), exogenous promoter, 2A “like” skip sequences, the “placeholder” TCR and an additional PA sequence. The TRAC domain within the “placeholder” TCR contains the landing pad 3′ excision site. Following monoallelic integration into the desired locus a second targeting vector is designed to be integrated following the excision of the landing pad using a guide RNA targeting the excision sites. The secondary targeting vector contains left and right homology arms (HA), an exogenous promoter, 2A “like” skip sequences and the therapeutic TCR. The TCRα chain of the therapeutic TCR contains a truncated TRAC domain. The full-length TRAC domain is reconstituted when the therapeutic TCR is knocked into the landing pad.

FIG. 6 describes a targeting strategy for the insertion of TCR “landing pad” into a genomic “safe-harbour” locus. In this scenario the 5′ and 3′ excision sequences correspond to region of B2M (Chr 15 44715435 to 44715475) containing a guide RNA recognition sequence. The excision sequence flanks the placeholder TCR. The flanking regions can be modified to contain any guide sequences of interest. The targeting vector contains left and right homology arms (HA) corresponding to the 300-1000 nucleotides of genomic DNA 5′ and 3′ from the integration site, excision domains, poly A signal (PA), exogenous promoter, 2A “like” skip sequences, the “placeholder” TCR and an additional PA sequence. Following monoallelic integration into the desired genomic location a second targeting vector is designed to be integrated following the excision of the landing pad using a guide RNA targeting the excision sites. The secondary targeting vector contains left and right homology arms (HA), 2A “like” skip sequences and the therapeutic TCR.

FIG. 7 describes the AAV targeting vector for the used for the insertion of the TCR landing pad with TRAC guide RNAs into PTPRC exon 33. The left and right homology arms correspond to Chromosome one 198755130-198756201 and Chromosome one 198,756, 132-198,757,230 (Ensembl release 104—May 20210) respectively. The guide RNA sequence within the LHA has been mutated to prevent cutting of the repair template. The MAGE-A10 TCR ADB796 is expressed via an exogenous promoter (EF1a short). The landing pad TCR cassette contains two guide RNA sequences derived from TRAC Exon 1. The 5′ site is site is contained within a truncated TRAC domain (Chr14: 22,547,508-22,547,560) and the 3′ site is located within the TRAC domain of the MAGE-A10 TCR ADB796. Editing with a single guide RNA permits excision of the landing pad TCR.

FIG. 8 describes the AAV targeting vector for the used for the insertion of the TCR landing pad with TRAC guide RNAs into the AAVS1 safe harbour site PPP1r12C intron 1. The left and right homology arms correspond to Chr 19:55115776-55116775 and Chr 19:55114775-55115775 (Ensembl release 104—May 2021) respectively. The MAGE-A10 TCR ADB796 is expressed via an exogenous promoter (EF1a short). The landing pad TCR cassette contains two guide RNA sequences derived from TRAC Exon 1. The 5′ site is site is contained within a truncated TRAC domain (Chr14: 22,547,508-22,547,560) and the 3′ site is located within the TRAC domain of the MAGE-A10 TCR ADB796. Editing with a single guide RNA permits excision of the landing pad TCR. In an alternative variation to that shown, the position of the alpha and beta chains may be the other way around.

FIG. 9 describes the targeting vector for the insertion of a therapeutic TCR (MAGE-A4/B2 ADB959) into the TCR landing pad within PTPRC exon 33. The left homology arm homology arm targets Chr1: 198,754,605-198,756,226 (Ensembl release 104—May 2021) and the right homology arm targets the TCRα TRAC domain BGH polyA and genomic DNA corresponding to Chr 1:198,756, 132-198,757,230 (Ensembl release 104—May 2021). The nucleotide sequence within the TRAC domain of ABD959 has been mutated to prevent cleavage by the guide RNA used to excise the placeholder TCR. The design of the targeting vector allows a 2A like skip sequence tagged TCR to be inserted into in frame with PTPRC Exon 33.

FIG. 10 describes the targeting vector for the insertion of a therapeutic TCR (MAGE-A4/B2 ADB959) into the TCR landing pad within PPP1r12C intron 1. The left homology arm homology arm targets Chr 19: 55, 115, 701-55, 117, 349 (Ensembl release 104—May 2021) and the right homology arm targets the TCRα TRAC domain BGH polyA and genomic DNA corresponding to Chr19: 55,114,725-55, 115,825 (Ensembl release 104—May 2021). The nucleotide sequence within the TRAC domain of ABD959 has been mutated to prevent cleavage by the guide RNA used to excise the placeholder TCR. The expression of ADB959 is regulated via an exogenous promoter.

FIG. 11 shows an overview of the landing pad strategy. Placeholder TCR is knocked-in to iPSC cells to support differentiation into CD8+ T cells. Following differentiation, iT cells are gene-edited to exchange the placeholder TCR for a patient specific therapeutic TCR.

FIG. 12 shows an editing strategy of the invention. FIG. 12A shows the generation of a novel universal cell bank using knock-in of the placeholder TCR (A2M10) cassette into the PPP1R12C (AAVS1) locus using the rAAV repair template ADB00794_001. B2M guide sequences for TCR excision are highlighted. TCRα and TCRβ chains are separated by P2A skip sequence. FIG. 12B shows the replacement of the placeholder TCR (A2M10) cassette with the exchange TCR (A2M4) cassette using rAAV repair template ADB01032_026. TCRα and TCRβ chains are separated by P2A skip sequence.

FIG. 13 shows the expression of A2M10 placeholder TCR in early-stage iT cell progenitor lines differentiated from a monoallelic placeholder TCR knock-in iPSC clone (15F2_AAVS1^{-/A2M10LP}) and from a biallelic placeholder TCR knock-in iPSC clone (16D5_AAVS1^{A2M10PL/A2M10LP}).

FIG. 14 shows the expression of A2M10 placeholder TCR and exchange for A2M4 TCR in early-stage 15F2_AAVS1^{-/A2M10LP} progenitor iT cells following CRISPR-Cas9-based gene editing. A) Cells were mock-electroporated and transduced with the A2M4 rAAV repair template at multiplicity of infection (MOI) of 5000 vg/cell. B) iT progenitor cells were electroporated with a ribonucleoprotein (RNP) complex targeting the B2M target sites in the placeholder TCR transgene cassette. C) iT progenitor cells were electroporated with an RNP complex targeting the B2M target sites of the placeholder transgene and transduced with rAAV encoding the A2M4 repair template at an MOI of 5000 vg/cell. D) Cells were electroporated with an RNP complex targeting the B2M target sites of the placeholder transgene, transduced by with rAAV encoding the ADB01032_026 A2M4 repair template at an MOI of 5000 vg/cell, and treated with 0.3 μM of M3814. TCR expression was analysed by flow cytometry 72 h post-electroporation.

FIG. 15 shows the quantification of the placeholder TCR gene editing outcomes in early-stage 15F2 AAVS1^{-/A2M10LP} progenitor iT cells following CRISPR-Cas9-based gene editing. A) Loss of A2M10Vβ13.2 placeholder TCR expression as a measure of CRISPR Cas9 knockout efficiency. B) Measurement of the A2M10 Vβ13.2 placeholder TCR replacement by the A2M4 Vα24 TCR. C) Frequency of A2M4 Vα24TCR expression in live progenitor iT cells. D) Frequency of A2M10 Vβ13.2 placeholder TCR expression in live progenitor iT cells.

FIG. 16 shows the expression of A2M10 placeholder TCR and A2M4 TCR in early-stage 16D5_AAVS1^{A2M10LP/A2M10LP} progenitor iT cells following CRISPR-Cas9-based gene editing. A) Cells were mock-electroporated and transduced with the A2M4 rAAV-ADB01032_026 repair template at MOI of 5000 vg/cell. B) iT progenitor cells were electroporated with an RNP complex targeting the B2M target sites in the placeholder TCR transgene cassette. C) iT progenitor cells were electroporated with an RNP complex targeting the B2M target sites of the placeholder transgene and transduced with rAAV encoding the A2M4 repair template (ADB01032_026) at an MOI of 5000 vg/cell. D) Cells were electroporated with an RNP complex targeting the B2M target sites of the placeholder transgene, transduced by with rAAV encoding the A2M4 repair template at an MOI of 5000 vg/cell, and treated with 1 μM of M3814. TCR expression was analysed by flow cytometry 72 h post-electroporation.

FIG. 17 shows the quantification of the placeholder TCR gene editing outcomes in early-stage 16D5_AAVS1^{A2M10LP/A2M10LP} progenitor iT cells following CRISPR-Cas9-based gene editing. A) Loss of A2M10 placeholder TCR expression as a measure of CRISPR Cas9 knockout efficiency. B) Measurement of the A2M10 Vβ13.2 placeholder TCR replacement by the A2M4 Vα24 TCR. C) Frequency of A2M4 Vα24 TCR expression in live progenitor iT cells. D) Frequency of A2M10 Vβ13.2 placeholder TCR expression in live progenitor iT cells.

FIG. 18 shows the expression of A2M10 placeholder TCR and A2M4 TCR in CD3/CD28 activated late-stage 15F2_AAVS1^{-/A2M10LP} progenitor iT cells following CRISPR-Cas9-based gene editing. A) Cells were mock-electroporated and transduced with the A2M4 rAAV-ADB01032_026 repair template at MOI of 5000 vg/cell. B) iT progenitor cells were electroporated with an RNP complex targeting the B2M target sites in the placeholder TCR transgene cassette. C) iT progenitor cells were electroporated with an RNP complex targeting the B2M target sites of the placeholder transgene and transduced with rAAV encoding the A2M4-ADB01032_026 repair template at an MOI of 5000 vg/cell. D) Cells were electroporated with an RNP complex targeting the B2M target sites of the placeholder transgene, transduced by with rAAV encoding the

A2M4 repair template at an MOI of 5000 vg/cell, and treated with 0.6 μM of M3814. TCR expression was analysed by flow cytometry 72 hours post-electroporation.

FIG. 19 shows the quantification of the placeholder TCR gene editing outcomes in CD3/CD28 activated late-stage 15F2_AAVS1^{-/A2M10LP} progenitor iT cells following CRISPR-Cas9-based gene editing. A) Loss of A2M10 placeholder TCR expression as a measure of CRISPR Cas9 knockout efficiency. B) Measurement of the A2M10 Vβ13.2 placeholder TCR replacement by the A2M4 Vα24 TCR. C) Frequency of A2M4 Vα24TCR expression in live progenitor iT cells. D) Frequency of A2M10 Vβ13.2 placeholder TCR expression in live progenitor iT cells.

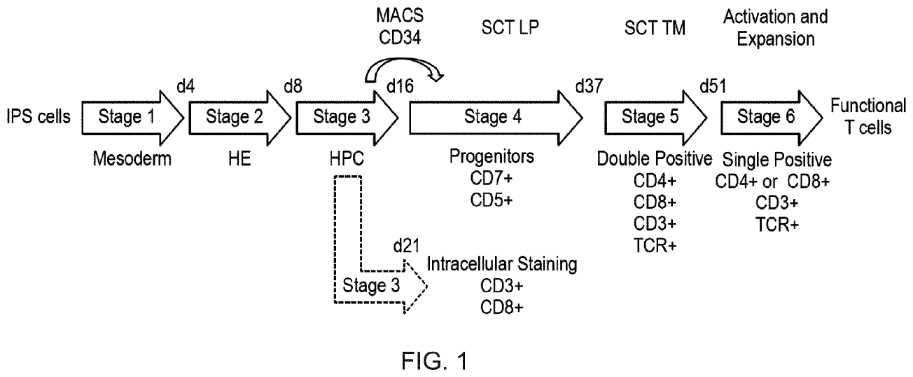

### DETAILED DESCRIPTION

This invention relates to the production of immune cells expressing a therapeutic antigen receptor, such as a T cell receptor (TCR). Immune cells are generated from iPSCs that comprise a heterologous expression cassette that expresses a “placeholder” production TCR. The expression of the production TCR in the cells avoids differentiation arrest and allows the generation of mature immune cells, for example CD3+ T cells. The immune cells may then be primed using the heterologous expression cassette as a “landing pad” for an expression construct comprising a nucleotide sequence encoding a therapeutic antigen receptor. The expression construct is inserted into the genome of the immune cells at the site of the heterologous expression cassette. For example, the expression construct may replace the heterologous expression cassette in the immune cells. The expression construct replaces the heterologous expression cassette in the immune cells, which then express the therapeutic antigen receptor. Immune cells produced as described herein may be useful in immunotherapy.

For example, methods described herein may be useful in the rapid generation of immune cells for the treatment of cancer in a patient. The therapeutic antigen receptor expressed by the immune cells may be selected as being reactive with the cancer cells in a patient. The antigen receptor may for example be a TCR or other antigen receptor expressed by tumour infiltrating lymphocytes (TILs) obtained from the patient or may be an antigen receptor known to be reactive with a tumour antigen identified as being expressed by the cancer cells in the patient. An expression construct comprising a nucleotide sequence encoding the antigen receptor may be used to replace the heterologous expression cassette to generate immune cells that specifically reactive with cancer cells in the patient and may be useful for the treatment of cancer in the patient.

Immune cells suitable for use as described herein include T cells, such as αβ+ T cells, γδ+ T cells, mucosal associated invariant (MAIT) T cells and NK T cells.

T cells (also called T lymphocytes) are white blood cells that play a central role in cell-mediated immunity. T cells can be distinguished from other lymphocytes by the presence of a T cell receptor (TCR) on the cell surface. There are several types of T cells, each type having a distinct function.

T helper cells (TH cells) are known as CD4+ T cells because they express the CD4 surface glycoprotein. CD4+ T cells play an important role in the adaptive immune system and help the activity of other immune cells by releasing T cell cytokines and helping to suppress or regulate immune responses. They are essential for the activation and growth of CD8+ T cells. CD8+ T cells (Tc cells, CTLs, killer T cells, CD8+ T cells) express the CD8 surface glycoprotein. CD8⁺ T cells act to destroy virus-infected cells and tumour cells. Most CD8⁺ T cells express TCRs that can recognise a specific antigen displayed on the surface of infected or damaged cells by a class I MHC molecule. Specific binding of the TCR and CD8 glycoprotein to the antigen and MHC molecule leads to T cell-mediated destruction of the infected or damaged cells.

T cells produced as described herein may be double positive CD4+CD8+ T cells or single positive CD4+ or CD8+ T cells. Preferred T cells include CD8+ T cells.

Preferred T cells may include TCR αβ+ T cells. TCR αβ+ T cells produced as described herein may be mature CD3+ T cells. For example, the T cells may have a αβTCR+ CD3+ CD45+ CD28+ phenotype.

In the methods described herein, immune cells are primed for therapeutic use by the insertion of an expression construct encoding a therapeutic TCR at the site of a heterologous expression cassette encoding a production TCR. For example, the heterologous expression cassette encoding the production TCR may be replaced with the expression construct encoding the therapeutic TCR.

TCRs are disulphide-linked membrane anchored heterodimeric proteins that comprise highly variable alpha (α) and beta (β) chains or delta (δ) and (γ) gamma chains expressed as a complex with invariant CD3 chain molecules. T cells expressing these types of TCRs may be referred to as αβ (or α:β) T cells and δΓ (or δ:γ) T cells.

TCRs bind specifically to major histocompatibility complexes (MHC) on the surface of cells that display a peptide fragment of a target antigen. For example, TCRs may bind specifically to a major histocompatibility complex (MHC) on the surface of cancer cells that displays a peptide fragment of a tumour antigen. Alternatively, TCRs may recognise specific antigen or peptide thereof independent of presentation by MHC. T cells comprising such TCRs may be produced according to the methods of the present invention. An MHC is a set of cell-surface proteins which allow the acquired immune system to recognise ‘foreign’ molecules. Proteins are intracellularly degraded and presented on the surface of cells by the MHC. MHCs displaying ‘foreign’ peptides, such a viral or cancer associated peptides, are recognised by T cells with the appropriate TCRs, prompting cell destruction pathways. MHCs on the surface of cancer cells may display peptide fragments of tumour antigen i.e. an antigen which is present on a cancer cell but not the corresponding non-cancerous cell. T cells which recognise these peptide fragments may exert a CD8+ effect on the cancer cell.

The production and therapeutic TCRs described herein are not naturally expressed by the iPSCs or immune cells described herein (i.e. the TCRs is exogenous or heterologous). Suitable heterologous TCRs may bind specifically to class I or II MHC molecules displaying peptide fragments of a target antigen. The production and therapeutic TCRs may be synthetic or artificial TCRs i.e. TCRs that do not exist in nature.

The production TCR and the therapeutic TCR may be encoded by heterologous nucleic acids. The term “heterologous” refers to a polypeptide or nucleic acid that is foreign to a particular biological system, such as a host cell, and is not naturally present in that system. A heterologous polypeptide or nucleic acid may be introduced to a biological system by artificial means, for example using recombinant techniques. For example, a heterologous nucleic acid encoding a polypeptide may be inserted into a suitable expression construct which is in turn used to transform a host cell to produce the polypeptide. A heterologous polypeptide or nucleic acid may be synthetic or artificial or may exist in a different biological system, such as a different species or cell type. An endogenous polypeptide or nucleic acid is native to a particular biological system, such as a host cell, and is naturally present in that system. A recombinant polypeptide is expressed from a heterologous nucleic acid that has been introduced into a cell by artificial means, for example using recombinant techniques. A recombinant polypeptide may be identical to a polypeptide that is naturally present in the cell or may be different from the polypeptides that are naturally present in that cell.

A coding sequence for a TCR, such as a production TCR, or therapeutic antigen receptor, may comprise coding sequences for the alpha (α) and beta (β) chains or delta (δ) and (γ) gamma chains that are separated by a nucleotide sequence encoding a self-cleaving peptide, such as a 2A peptide. This allows the stochiometric expression of both chains from a single transcript.

The heterologous expression cassette is a recombinant nucleic acid incorporated into the genome of the immune cell and its precursors. The heterologous expression cassette supports the production of mature immune cells by allowing the expression of the production TCR. For example, expression of the production TCR allows the differentiation of progenitor cells into T cells. Following production of mature immune cells, the heterologous expression cassette forms a “landing pad” that allows the expression construct to replace the heterologous expression cassette at same site in the genome. The expression cassette may comprise any suitable nucleic acid sequence, as described below. Preferred heterologous expression cassettes may be excised with a single guide RNA to completely remove the production TCR.

A production TCR is expressed by the immune cell and its precursors during its production. Differentiation into immune cells is arrested in cells lacking TCR expression. Expression of the production TCR may facilitate the production of mature immune cells, such as T cells. For example, the expression of the production TCR in the immune cell may induce or promote the surface expression of CD3 and allow differentiation into lymphopoietic lineages, such as CD3+ T cells. After differentiated CD3+ immune cells have been generated, the therapeutic antigen receptor may be inserted at the site of the production TCR. For example, the production TCR may be replaced in the cells with the therapeutic antigen receptor.

Suitable production TCRs include any TCR that supports T cell differentiation and surface expression of CD3 and prevents differentiation arrest. Unlike the therapeutic antigen receptor, the production TCR is not patient-specific and does not mediate any therapeutic effect of the immune cells in a patient.

In some embodiments, the production TCR may lack binding activity. For example, the production TCR may be functionally inert and may lack TCR functions other than promoting T cell differentiation and surface CD3 expression. This may be useful for example in reducing the need to isolate or purify T cells expressing the therapeutic antigen receptor following replacement of the production TCR. Suitable functionally inert production TCRs may for example lack one or both TCR variable regions. For example, a production TCR may lack the α chain variable region and/or the β chain variable region.

In some embodiments, the production TCR may bind to class 1 MHCs displaying fragments of antigens of no clinical relevance. For example, the production TCR may display no binding or substantially no binding to tumour antigens or other clinically relevant antigens and may not bind to cancer cells in a patient. In some embodiments, a production TCR may be engineered to reduce or abolish its affinity or avidity for an antigen.

Suitable production TCRs may comprise various different combinations of α and β chains or variants thereof, or gamma and delta chains, or variants thereof. The production TCRs may be human or non-human, for example murine TCRs. For example, a production TCR may comprise or consist of (i) full-length α and β chains (ii) α and β constant domains (TRAC (P01848-1) and TRBC (P01850-1)) (iii) a single chain αβ TCR (for example a TCR with the α and β chains linked by a peptide linker); (iv) a β chain and a chimeric chain comprising the variable and constant domains of an α chain fused to the transmembrane and cytoplasmic domains of a pre-α chain (v) a full-length β chain and a full-length pre-α chain (vi) a full-length β chain and a truncated pre-α chain with a 48 aa deletion at the C-terminus (Δ48) (vii) a fragment of a β chain comprising or consisting of residues 125-176 (P01850-1;TRBC1_human aa 125-176;) and a fragment of pre-α chain comprising or consisting of residues 126 to 281 (PTCRA_human aa 126-281 (A0A087WTE9-1); or (viii) the constant domain of a β chain and a full-length pre-α chain.

In some preferred embodiments, the production TCR may comprise or consist of (i) full-length α and β chains or (ii) a full-length β chain and a full-length pre-α chain.

In other preferred embodiments, the production TCR may comprise or consist of (i) a β chain and a chimeric chain comprising the variable, constant and transmembrane domains of an a chain fused to the cytoplasmic domain of a pre-α chain or (ii) a β chain and a chimeric chain comprising the variable and constant domains of an a chain fused to the transmembrane and cytoplasmic domains of a pre-α chain.

The amino acid and encoding nucleotide sequences of suitable α, pre α and β chains and domains thereof are well-known in the art.

Production TCRs suitable for use as described herein are readily available in the art and include MAGE-A10 αβ TCR clone 796 (SEQ ID NOs: 14 to 17; SEQ ID NO: 60); MR1 TCR MC.7.G5 clone-αβTCR

(TRAV38.2/DV8 TRAJ31 α-chain; TRBV25.1 TRBJ2.3 β-chain) (Crowther et al. 2020 Nature Immunology 21 178-185); invariant NKT αβ TCR (Vα24-Jα18 paired with Vβ11); γδ TCR Vγ5Vδ1 or Vγ1Vδ4 (Ribot et al. (2021) Nature Rev Immunology 21 221-232); γδ TCR Vγ9JPVδ2 (Ravens et al. (2018) Fron Immunol 9 510; Di Lorenzo et al. (2019) Sci Data 6 115; Xu et al. 2021 Cell Mol Immunol. 2021 February;18 (2): 427-439); and HLA-E restricted TCRs against viral antigens, such as CMV and HIV (Yang et al (2021) Sci Immunol 6 57; Pietra et al. (2003) PNAS USA 100 (19) 10896-10901). In some preferred embodiments, a MAGE-A10 αβ TCR clone 796 with the α chain amino acid sequence of SEQ ID NO: 14 and the β chain amino acid sequence of SEQ ID NO: 15 or a MAGE-A10 αβ TCR clone 794 with the a chain amino acid sequence of SEQ ID NO: 67 and the β chain amino acid sequence of SEQ ID NO: 72 may be employed. A suitable α chain may be encoded by the nucleotide sequence of SEQ ID NO: 16 or SEQ ID NO: 66 and a suitable β chain may be encoded by the nucleotide sequence of SEQ ID NO: 17 or SEQ ID NO: 71.

Suitable nucleotide sequences are well known in the art. The heterologous expression cassette may comprise one or more nucleic acids encoding the production TCR. A heterologous nucleic acid encoding a TCR may encode all the sub-units of the receptor. Preferably, the chains of the production TCR are expressed in a single transcript. For example, a nucleic acid encoding a TCR may comprise a first nucleotide sequence encoding a TCR α chain and a second nucleotide sequence encoding a TCR β chain or a first nucleotide sequence encoding a TCR δ chain and a second nucleotide sequence encoding a TCR γ chain. The coding nucleic acid may further comprise a nucleotide sequence encoding a 3′ poly (A) sequence. Suitable nucleotide sequences encoding poly (A) sequences are shown in SEQ ID NO: 4, SEQ ID NO: 10 and SEQ ID NO: 40.

In some embodiments, the heterologous expression cassette may comprise one or more nucleic acids encoding a CD3 chimeric fusion receptor instead of a production TCR.

A self-cleaving peptide coding sequence may be located between the first nucleotide sequence encoding the TCRα or TCRδ chain and the second nucleotide sequence encoding the TCRβ chain or TCRγ chain. The self-cleaving peptide causes cleavage of the nascent peptide chain during translation through ribosome skipping and separates the chains of the TCR. Suitable 2A peptides may include T2A, P2A, E2A and F2A peptides (Poddar et al (2018) supra; Kim et al (2011) PLOS ONE 6, e18556). A preferred 2A peptide may comprise the amino acid sequence of SEQ ID NO: 1, SEQ ID NO: 29, SEQ ID NO: 31 or SEQ ID NO: 69. A self-cleaving peptide coding sequence may comprise the nucleotide sequence of SEQ ID NO: 2, SEQ ID NO: 28, SEQ ID NO: 30, SEQ ID NO: 70 and SEQ ID NO: 80.

A nucleic acid encoding a furin cleavage site may be located adjacent the self-cleaving peptide coding sequence. This may be useful in removing self-cleaving peptide residues from the TCR chains. Suitable furin cleavage sites and coding sequences are shown in SEQ ID Nos 11, 12, 24 to 27 and 68.

The expression cassette may further comprise a promoter operably linked to the coding sequence for the production TCR. The promoter may drive the expression of the production TCR in the immune cell. Suitable promoters include constitutive promoters, such as the SV40, CMV, UBC, EF1A, EF1AS, PGK, JeT, MND or CAGG promoter or variants thereof. The nucleotide sequences of suitable EF1A promoters are shown in SEQ ID NOs: 3, 36 and 65. Examples of nucleotide sequences of expression cassettes for the A2M10 production TCR are shown in SEQ ID NOs: 58 and 76.

The heterologous expression cassette comprises a targeting site. A targeting site is a nucleotide sequence that mediates insertion of the expression construct at the site of the expression cassette. For example, a targeting site may mediate the replacement of the heterologous expression cassette in the immune cell genome with the expression construct. In some embodiments, the targeting site may be located upstream of the constitutive promoter in the heterologous expression cassette. Preferably, the targeting site may be located at the 5′ end of the cassette. In other embodiments, the targeting site may be located within the coding sequence for the production TCR.

In some embodiments, the heterologous expression cassette may comprise 5′ and 3′ targeting site. For example, the heterologous expression cassette may be cleaved at the 5′ and 3′ targeting sites and excised from the genome of the immune cell. In some embodiments, the nucleotide sequences of the targeting site or the 5′ and 3′ targeting sites are unique in the genome of the immune cell.

In some embodiments, the 5′ targeting site may be located upstream of the constitutive promoter in the heterologous expression cassette. Preferably, the 5′ targeting site is located at the 5′ end of the cassette.

In some embodiments, the 3′ targeting site may be located downstream of the coding sequence or at the 3′ end of the coding sequence. Preferably, the 3′ targeting site is located at the 3′ end of the cassette.

One of the 5′ and 3′ targeting sites may be located within the coding sequence for the production TCR.

The choice of 5′ and 3′ targeting sites may depend on the technique selected for the replacement of the expression cassette. For example, suitable 5′ and 3′ targeting sites may include CRISPR guide RNA recognition sequences for CRISPR mediated replacement, IoxP sites for CRE-LOXP mediated replacement, FRT sites for FLP-FRT mediated replacement; and recognition sites for site specific nucleases, such as Transcription activator-like effector nucleases (TALENs)

The expression construct may be inserted into the cell genome at the site of the expression cassette using any suitable technique. In some preferred embodiments, the expression cassette may be replaced by the expression construct using CRISPR mediated replacement techniques. For example, the 5′ and 3′ targeting sites may include CRISPR guide RNA recognition sequences. Suitable guide RNA recognition sequences may for example contain 19 to 21 nucleotides. Preferably, the guide RNA recognition sequences are unique within the immune cell genome to avoid off-target effects. Examples of suitable guide RNA recognition sequences include SEQ ID NOs: 5 to 9. Methods of designing suitable guide RNA recognition sequences for use in CRISPR mediated replacement are well-established in the art.

In some embodiments, the targeting site or one of the 5′ and 3′ targeting sites, preferably the 3′ targeting site, may be located within the coding sequence for production TCR, for example within the TCR chain constant region coding sequence, such as the TCRα chain constant region coding sequence (TRAC) or the TCRβ chain constant region coding sequence (TRBC). An example of a preferred 5′ targeting site comprising sequence from the MAGE-A10 c796 TCR alpha chain (TRAC) coding sequence is shown in SEQ ID NO: 13. The corresponding 3′ targeting site is located within the MAGE-A10 c796 TCR alpha chain coding sequence of the expression cassette (see SEQ ID NO: 16).

In other embodiments, the targeting site or one of the 5′ and 3′ targeting sites, preferably the 3′ targeting site, may comprise a nucleotide sequence from the gene locus into which the expression cassette is inserted.

In some embodiments, the 5′ and 3′ targeting sites may comprise the same nucleotide sequence. This may facilitate the removal of the heterologous expression cassette, for example using a single guide RNA. For example, the same targeting site may be positioned at the both the 5′ and 3′ ends of the expression cassette. For example, the expression cassette may comprise a TRAC or TRBC sequence at its 5′ and 3′ ends.

An example of a nucleotide sequence of a plasmid for the insertion of the A2M10 production TCR is shown in SEQ ID NO: 59.

The heterologous expression cassette may be incorporated into the genome of the immune cell. In some embodiments, the heterologous expression cassette may be incorporated into the genome of the immune cell within a gene locus that comprises an endogenous promoter. For example, the heterologous expression cassette may be integrated within or immediately adjacent an exon of a gene in the gene locus, preferably the last exon of a gene in the locus. Integration retains the natural reading frame of the gene, so that the expression of the therapeutic antigen receptor is driven by the endogenous promoter following replacement of the heterologous expression cassette with the expression construct as described herein. Suitable gene loci may be active in differentiated T cells and may include TRAC, PTPRC, EEF1A1, CD3E, CD3D, CD3G CD8A, and CD2.

In other embodiments, the heterologous expression cassette may be incorporated into the genome of the immune cell within a safe harbour locus. This allows the expression of the therapeutic antigen receptor to be driven by a constitutive promoter contained in the expression construct. A suitable expression construct may for example comprise a nucleic acid encoding a poly (A) sequence and a constitutive promoter. Suitable safe harbour loci include AAVS1 and are shown in Table 1.

A therapeutic antigen receptor is expressed by the immune cell following the insertion of the expression construct at the site of the expression cassette. For example the receptor may be expressed following the replacement of the expression cassette with the expression construct.

The therapeutic antigen receptor mediates the therapeutic effect of the immune cells. Preferably, the therapeutic antigen receptor binds to cancer cells in a patient. For example, a therapeutic T antigen receptor may bind specifically to class I or II MHC molecules displaying peptide fragments of a tumour antigen expressed by the cancer cells in a cancer patient. In some embodiments, the therapeutic T antigen receptor may recognise a target antigen or a peptide fragment of a target antigen on the cancer cell independently of MHC presentation. Tumour antigens expressed by cancer cells in the cancer patient may identified using standard techniques. Preferred tumour antigens include NY-ESO1, PRAME, alpha-fetoprotein (AFP), MAGE A4, MAGE A1, MAGE A10 and MAGE B2, most preferably NY-ESO-1, MAGE-A4 and MAGE-A10.

In some embodiments, a tumour antigen in a patient may be identified and a therapeutic antigen receptor that binds to the tumour antigen selected for use in the expression construct.

In some embodiments, the therapeutic antigen receptor may be a chimeric antigen receptor (CAR). CARs are artificial receptors that are engineered to contain an immunoglobulin antigen binding domain, such as a single-chain variable fragment (scFv). A CAR may, for example, comprise an scFv fused to a TCR CD3 transmembrane region and endodomain. An scFv is a fusion protein of the variable regions of the heavy (V_{H}) and light (V_{L}) chains of immunoglobulins, which may be connected with a short linker peptide of approximately 10 to 25 amino acids (Huston J. S. et al. Proc Natl Acad Sci USA 1988; 85 (16): 5879-5883). The linker may be glycine-rich for flexibility, and serine or threonine rich for solubility, and may connect the N-terminus of the V_{H} to the C-terminus of the V_{L}, or vice versa. The scFv may be preceded by a signal peptide to direct the protein to the endoplasmic reticulum, and subsequently the T cell surface. In the CAR, the scFv may be fused to a TCR transmembrane and endodomain. A flexible spacer may be included between the scFv and the TCR transmembrane domain to allow for variable orientation and antigen binding. The endodomain is the functional signal-transmitting domain of the receptor. An endodomain of a CAR may comprise, for example, intracellular signalling domains from the CD3 ζ-chain, or from receptors such as CD28, 41BB, or ICOS. A CAR may comprise multiple signalling domains, for example, but not limited to, CD3z-CD28-41BB or CD3z-CD28-OX40.

The CAR may bind specifically to a tumour-specific antigen expressed by cancer cells. For example, the T cells may be modified to express a CAR that binds specifically to a tumour antigen that is expressed by the cancer cells in a specific cancer patient. Tumour antigens expressed by cancer cells in the cancer patient may identified using standard techniques.

In other embodiments, the therapeutic antigen receptor may be an NK cell receptor (NKCR).

In other embodiments, the therapeutic antigen receptor may be a T cell receptor (TCR). TCRs are described elsewhere herein and may include αβTCR heterodimers and γδTCR heterodimers. Suitable heterologous TCR may bind specifically to class I or II MHC molecules displaying peptide fragments of a target antigen. For example, the T cells may be modified to express a heterologous TCR that binds specifically to class I or II MHC molecules displaying peptide fragments of a tumour antigen expressed by the cancer cells in a cancer patient. Tumour antigens expressed by cancer cells in the cancer patient may be identified using standard techniques. Preferred tumour antigens include NY-ESO1, PRAME, alpha-fetoprotein (AFP), MAGE A4, MAGE A1, MAGE A10 and MAGE B2, most preferably NY-ESO-1, MAGE-A4 and MAGE-A10.

In some preferred embodiments, a heterologous TCR may bind specifically to HLA-A*02:01 displaying the MAGEA4 peptide fragment GVYDGREHTV.

Suitable therapeutic TCRs may include unconventional TCRs, for example non-MHC dependent TCRs that bind recognize non-peptide antigens displayed by monomorphic antigen-presenting molecules, such as CD1 and MR1; NKT cell TCRs and intraepithelial lymphocyte (IEL) TCRs. In some embodiments, the therapeutic TCR may recognise target antigen or peptide fragment of target antigen on the cancer cell independently of MHC presentation.

Suitable therapeutic TCRs include patient-derived TCRs. For example, the therapeutic TCR may be a TCR from an immune cell, such as a tumour infiltrating lymphocyte (TIL), obtained from a donor individual. For example, a tumour in a patient may be profiled to identify tumour antigens expressed by the cancer cells in the tumour. TCRs reactive with the identified tumour antigens may be identified an inserted into expression constructs for use as therapeutic TCRs as described herein. In other embodiments, TCRs expressed by immune cells, such as tumour infiltrating lymphocytes (TILs) obtained from the patient may be sequenced and cloned into expression constructs. For example, a diverse repertoire of TCRs from the patient may be cloned into expression constructs for insertion into immune cells as described herein. Suitable techniques for obtaining the coding sequence of a TCR from an immune cell, such as a tumour infiltrating lymphocyte (TIL), obtained from the patient, and inserting it into an expression construct are established in the art. The target tumour antigen of a TCR from a TIL may be identified or may remain unidentified. The donor individual may be the same person as the recipient individual to whom the immune cells will be administered following production as described herein i.e. the therapeutic TCR may be derived from the patient to whom the immune cells are administered.

In some embodiments, a therapeutic TCR may be engineered to increase its affinity or avidity for a tumour antigen (i.e. an affinity enhanced TCR). An affinity enhanced TCR may comprise one or more mutations relative to a naturally occurring TCR, for example, one or more mutations in the hypervariable complementarity determining regions (CDRs) of the variable regions of the TCR α and β chains. These mutations may increase the affinity of the TCR for MHCs that display a peptide fragment of a tumour antigen expressed by cancer cells. Suitable methods of generated affinity enhanced TCRs include screening libraries of TCR mutants using phage or yeast display and are well known in the art (see for example Robbins et al J Immunol (2008) 180 (9): 6116; San Miguel et al (2015) Cancer Cell 28 (3) 281-283; Schmitt et al (2013) Blood 122 348-256; Jiang et al (2015) Cancer Discovery 5 901).

An example of an amino acid sequence of a A2M4 TCRα is shown in SEQ ID NO: 79 and an example of an amino acid sequence of a A2M4 TCRβ is shown in SEQ ID NO: 82. An example of an amino acid sequence of a therapeutic TCR is shown in SEQ ID NO: 62.

The expression construct is a recombinant nucleic acid that is incorporated into the genome of the immune cell at the site of the heterologous expression cassette. For example, the expression construct may replace the heterologous expression cassette. The expression construct comprises a coding sequence for the therapeutic TCR. The coding sequence for the therapeutic TCR may for example comprise a first nucleotide sequence encoding a TCRα chain and a second nucleotide sequence encoding a TCRβ chain or a first nucleotide sequence encoding a TCRγ chain and a second nucleotide sequence encoding a TCRδ chain. An example of a suitable first nucleotide sequence encoding a ADB959 TCRα chain is shown in SEQ ID NO: 33and SEQ ID NO: 38. An example of a suitable second nucleotide sequence encoding a ADB959 TCRβ chain is shown in SEQ ID NO: 32 and SEQ ID NO: 37. An example of a suitable first nucleotide sequence encoding a A2M4 TCRα chain is shown in SEQ ID NO: 78 and an example of a suitable second nucleotide sequence encoding a A2M4 TCRβ chain is shown in SEQ ID NO: 81.

The first and second nucleotide sequences may be located in a single open reading frame and may be separated by a third nucleotide sequence encoding a self-cleaving peptide, such as a 2A peptide and/or a furin linker. Suitable self-cleaving peptides are described in more detail above.

An example of an amino acid sequence (PTPRC Exon 33_T2A_ADB959_TCRβ _P2A_TCRα) encoded by an expression construct is shown in SEQ ID NO: 34. The amino acid sequence includes T2A and P2A sequences to separate the ADB959 TCR chains and the PTPRC exon sequence.

In some embodiments, the expression construct may further comprise a promoter operably linked to the coding sequence for the therapeutic antigen receptor. The promoter may drive the expression of the therapeutic antigen receptor in the immune cell. Suitable promoters include constitutive promoters, such as the SV40, CMV, UBC, EF1A, EF1AS, PGK, JeT, MND or CAGG promoter or variants thereof. The nucleotide sequences of suitable EF1A promoters are shown in SEQ ID NO: 3 and SEQ ID NO: 36. The expression construct may be inserted into the genome of the immune cell at the same site as the heterologous expression cassette. For example, the heterologous expression cassette may be replaced in the immune cell by the expression construct. This primes the immune cell for therapeutic use in an individual. Preferably, the heterologous expression cassette is completely excised, such that no sequence from the heterologous expression cassette remains in the immune cell following replacement. Any suitable technique may be used to achieve the replacement of the heterologous expression cassette with the expression construct.

An example of a nucleotide sequence of a plasmid for the insertion of the A2M4 therapeutic TCR is shown in SEQ ID NO: 61.

Suitable techniques include HR mediated gene replacement techniques, such as CRISPR/Cas9-based techniques and recombinase-mediated gene replacement techniques, such as Cre-Lox, FLP-FRT, or phiC31 integrase techniques. Suitable techniques are well known in the art (see for example Yamamoto et al Chromosoma. (2018) 127 (4): 405-420; Sakuma et al; (2016) Nat Protoc 11 (1) 118-133).

In some preferred embodiments, the expression cassette may be replaced by HR mediated target gene replacement. For example, the heterologous expression cassette may be replaced by a method comprising:
introducing into the immune cell a nucleic acid molecule, such as a DNA molecule, comprising the expression construct flanked by 5′ and 3′ homology arms, wherein the 5′ and 3′ homology arms are complementary to the nucleotide sequences at the 5′ and 3′ ends of the heterologous expression cassette and/or the genomic sequence flanking the heterologous expression cassette,
such that the expression construct replaces the expression cassette in the genome of the immune cell.

The homology arms mediate replacement of the heterologous expression cassette with the expression construct following cleavage of the heterologous expression cassette at the 5′ and 3′ targeting sites. Suitable homology arms may comprise sequence of 300 to 500 nucleotides that is complementary to the nucleotide sequence of the heterologous expression cassette and/or the genomic sequence flanking the heterologous expression cassette that is 5′ or 3′ respectively of the 5′ and 3′ targeting sites, such that the homology arms are complementary to the sequence at the gene locus or safe harbour locus following the removal or excision of the sequence of the heterologous expression cassette between the targeting sites.

In some embodiments, the HR mediated target gene replacement may be mediated by a programmable nuclease, for example, site-specific nucleases, such as zinc-finger nucleases (ZFNs), transcription activator-like effector nucleases (TALENs) and meganucleases or RNA guided nucleases, such as clustered regularly interspaced short palindromic repeat (CRISPR) nucleases.

Zinc-finger nucleases (ZFNs) comprise one or more Cys₂-His₂ zinc-finger DNA binding domains and a cleavage domain (i.e., nuclease). The DNA binding domain may be engineered to recognize and bind to any nucleic acid sequence using conventional techniques (see for example Qu et al. (2013) Nucl Ac Res 41 (16): 7771-7782). The use of ZFNs to introduce mutations into target genes is well-known in the art (see for example, Beerli et al Nat. Biotechnol.2002; 20:135-141; Maeder et al Mol. Cell. 2008; 31:294-301; Gupta et al Nat. Methods. 2012; 9:588-590) and engineered ZFNs are commercially available (Sigma-Aldrich (St. Louis, MO).

Transcription activator-like effector nucleases (TALENs) comprise a nonspecific DNA-cleaving nuclease fused to a DNA-binding domain comprising a series of modular TALEN repeats linked together to recognise a contiguous nucleotide sequence. The use of TALEN targeting nucleases is well known in the art (e.g. Joung & Sander (2013) Nat Rev Mol Cell Bio 14:49-55; Kim et al Nat Biotechnol. (2013); 31:251-258. Miller J C, et al. Nat. Biotechnol. (2011) 29:143-148. Reyon D, et al. Nat. Biotechnol. (2012); 30:460-465).

Meganucleases are endodeoxyribonucleases characterized by a large recognition site (double-stranded DNA sequences of 12 to 40 base pairs); as a result this site generally occurs only once in any given genome (see for example Silva et al. (2011) Curr Gene Ther 11 (1): 11-27).

CRISPR targeting nucleases (e.g. Cas9) complex with a guide RNA (gRNA) to cleave genomic DNA in a sequence-specific manner. The crRNA and tracrRNA of the guide RNA may be used separately or may be combined into a single RNA to enable site-specific mammalian genome cutting at the 5′ and 3′ targeting sites of the expression cassette. The use of CRISPR/Cas9 systems to introduce double strand breaks into a gene locus, for example as a way of introducing transgenes is well known in the art (see for example Cader et al Nat Immunol 2016 17 (9) 1046-1056, Hwang et al. (2013) Nat. Biotechnol 31:227-229; Xiao et al., (2013) Nucl Acids Res 1-11; Horvath et al., Science (2010) 327:167-170; Jinek M et al. Science (2012) 337:816-821; Cong L et al. Science (2013) 339:819-823; Jinek M et al. (2013) eLife 2: e00471; Mali P et al. (2013) Science 339:823-826; Qi LS et al. (2013) Cell 152:1173-1183; Gilbert LA et al. (2013) Cell 154:442-451; Yang H et al. (2013) Cell 154:1370-1379; and Wang H et al. (2013) Cell 153:910-918).

In some preferred embodiments, the targetable nuclease is a Cas endonuclease which is expressed in the immune cells in combination with a guide RNA that targets the Cas endonuclease to cleave the heterologous expression cassette at the 5′ and 3′ targeting sites.

Preferably, the HR mediated target gene replacement is mediated by CRISPR/Cas9. For example, a DNA double strand break (DSB) at a target site may be induced by a CRISPR/Cas9 system and the repair of the DSB may introduce the expression construct into the cell genome at the target site or the nucleic acid may be introduced using an rAAV vector (AAV mediated gene editing; Hirsch et al 2014 Methods Mol Biol 1114 291-307). For example, the heterologous expression cassette may be replaced by a method comprising;
introducing into the immune cell a nucleic acid molecule, such as a DNA molecule, comprising the expression construct flanked by 5′ and 3′ homology arms, wherein the 5′ and 3′ homology arms are complementary to the nucleotide sequences at the 5′ and 3′ ends of the heterologous expression cassette and/or the genomic sequence flanking the heterologous expression cassette, and
introducing into the immune cell a CRISPR/Cas9 targeting the 5′ and 3′ targeting sites,
such that the expression construct replaces the expression cassette in the genome of the immune cell.

Suitable homology arms are described above and may comprise a sequence of 300 to 500 nucleotides that is complementary to the nucleotide sequences at the 5′ and 3′ ends of the heterologous expression cassette and/or the genomic sequence flanking the heterologous expression cassette.

Suitable homology arms for a DNA molecule or targeting vector for an expression cassette in exon 33 of PTPRC are shown in SEQ ID NOS: 22 and 23. Suitable homology arms for a DNA molecule or targeting vector for an expression cassette in intron 1 of PPP1R12C (AAVS1) are shown in SEQ ID NOs: 35 and 41. Other suitable homology arms are shown in SEQ ID NOs: 64 and 74 or SEQ ID NOs: 77 and 83 or described elsewhere herein.

The DNA molecule comprising the expression construct may be a single-stranded DNA molecule. A suitable single-stranded DNA molecule may be contained in a recombinant adeno-associated virus (rAAV) vector. The single stranded DNA molecule may be introduced into the immune cell by transfecting the cell with the rAAV vector.

Suitable targeting sites, such as 5′ and 3′ targeting sequences, may comprise a nucleotide sequence complementary to the guide RNA of the CRISPR/Cas9. Suitable sequences may for example consist of 17 to 24 nucleotides. The targeting sites may further comprise additional nucleotide sequences flanking the complementary nucleotide sequence. This may be required for improved efficacy when removing the heterologous expression cassette. For example, a targeting site may comprise an additional 1 to 15 nucleotides, preferably about 12 nucleotides, at the 5′ and 3′ ends of the complementary nucleotide sequence.

In some embodiments, the 3′ targeting site may be a nucleotide sequence located within the coding sequence for the constant region of the TCRα chain within the expression cassette. For example, the 3′ targeting site may be the 3′ end of the sequence encoding the constant region of the TCRα chain. The 5′ targeting site have the same nucleotide sequence as the 3′ targeting site i.e. the 5′ targeting site may be a copy of the 3′ end of the sequence encoding the constant region of the TCRα chain. The 5′ targeting site may be located upstream of the promoter within the expression cassette.

CRISPR/Cas9 may be introduced directly into the cell as a protein and gRNA, for example within a lipid nanoparticle, or may be introduced as a nucleic acid encoding CRISPR/Cas9, such as an mRNA, plasmid or viral vector, that is then expressed in the cell. A nucleic acid that encodes CRISPR/Cas9 may be introduced into the immune cell by any convenient method, such as RNP electroporation.

Because they have the same sequence, a single guide RNA may target the CRISPR/Cas9 to both the 5′ and 3′ targeting sites, thereby cleaving the expression cassette at its 5′ and 3′ ends and excising it from the genome. Following excision of the sequence of the expression cassette between the 5′ and 3′ targeting sites, the 5′ and 3′ homology arms, which are complementary sequences at the gene locus or safe harbour locus outside the targeting sites, mediate the incorporation of the expression construct into the locus.

Suitable guide RNA sequences for CRISPR-Cas9 mediated gene replacement may be designed using standard techniques. For example, suitable guide RNA sequences to target TRAC1 include SEQ ID NOs: 5 and 6. Suitable guide RNA sequences to target the AAVS1 safe harbour sequence include SEQ ID NO: 7. Suitable guide RNA sequences to target exon 2 of B2M sequence include SEQ ID NO: 8. Suitable guide RNA sequences to target PTPRC sequence include SEQ ID NO: 9.

A method described herein may further comprise reducing or silencing expression of endogenous TCR in the cells, for example by inactivating an endogenous TCR gene or endogenous RAG1 or RAG2 gene. For example, the method may further comprise inactivating the endogenous TCRα (TRAC) chain gene or TCRβ (TRBC1 or 2) chain gene or the endogenous RAG1 or RAG2 gene. This may be useful in reducing or preventing off-target toxicity of the immune cell. The endogenous gene may be inactivated in the immune cell or a progenitor cell, such as an IPSC. For example, an endogenous gene may be inactivated in the IPSC before the heterologous expression cassette is incorporated.

Any suitable technique may be used to inactivate the endogenous TCR gene. Conveniently, the endogenous TCR gene is inactivated at the same time as the replacement of the heterologous expression cassette. In some preferred embodiments, the expression cassette is replaced using a CRISPR/Cas9 that targets sequence within the cassette encoding constant region of the TCRα chain. The CRISPR/Cas9 may also target sequence within the endogenous gene encoding the constant region of the TCRα chain. This may introduce one or more inactivating mutations into the endogenous TCRα chain constant region (TRAC) gene.

A method described herein may further comprise reducing or silencing expression of the class Il transcriptional activator (CIITA) and/or beta-2-microglobulin (B2M), for example by inactivating an endogenous B2M or CIITA gene. The endogenous gene may be inactivated in the immune cell or a progenitor cell, such as an IPSC. This may be useful in reducing alloreactive effects and improving the persistence of the immune cells in vivo. In some embodiments, a method described herein may further comprise expressing a heterologous B2M-HLA-E (mBE) and B2M-HLA-G (mBG) fusion protein in the immune cells. A construct comprising a heterologous nucleic acid encoding the fusion protein operably linked to a suitable promoter may be inserted into the immune cell or a progenitor cell, such as an IPSC. This may be useful in protecting the cells against allogeneic NK cell-mediated lysis.

The immune cells may display expression of the therapeutic TCR or therapeutic TCRs and no expression of an endogenous TCR.

Immune cells comprising the heterologous expression cassette may be produced by directed differentiation from induced pluripotent stem cells (IPSCs). For example, a method for producing an immune cell that comprises the heterologous expression cassette may comprise;
(i) transfecting an iPSC with a nucleic acid comprising a heterologous expression cassette, such that the cassette is integrated into the genome of the iPSC,
   wherein the expression cassette comprises;
   (a) a coding sequence for a production T cell receptor (TCR),
   (b) a constitutive promoter operably linked to the coding sequence, and
   (c) a targeting site, and
(ii) differentiating the iPSC into an immune cell comprising the expression cassette.

In some embodiments, the targeting site may be a 5′ targeting site and the cassette may further comprise a 3′ targeting site.

The heterologous expression cassette may be integrated into a target locus of the iPSC by a method comprising;
introducing into the iPSC a nucleic acid molecule, such as a DNA molecule, comprising the expression cassette flanked by 5′ and 3′ homology arms, wherein the 5′ and 3′ homology arms are complementary to the nucleotide sequences flanking an integration site in a target locus, and
introducing into the immune cell a CRISPR/Cas9 that targets the integration site in the target locus,
such that the expression cassette integrates into the genome of the immune cell at the integration site in the target locus.

Suitable homology arms are described above and may comprise a sequence of 300 to 500 nucleotides that is complementary to the nucleotide sequences flanking an integration site in the target locus. Homology arms may be designed for any target locus using standard techniques.

Suitable target loci are described above and shown in Table 1. In some embodiments the heterologous expression cassette may be integrated into exon 33 of PTPRC. Suitable homology arms for a DNA molecule or targeting vector for exon 33 of PTPRC are shown in SEQ ID NOS: 18 and 19. In other embodiments, the heterologous expression cassette may be integrated into intron 1 of PPP1R12C (AAVS1). Suitable homology arms for a DNA molecule or targeting vector for intron 1 of PPP1R12C (AAVS1) are shown in SEQ ID NOS: 20 and 21.

The DNA molecule comprising the heterologous expression cassette may be a single-stranded DNA molecule. A suitable single-stranded DNA molecule may be contained in a recombinant adeno-associated virus (rAAV) vector. The single stranded DNA molecule may be introduced into the immune cell by transfecting the cell with the rAAV vector.

Induced pluripotent stem cells (iPSCs) are pluripotent cells which are derived from non-pluripotent, fully differentiated donor or antecedent cells. iPSCs are capable of self-renewal in vitro and exhibit an undifferentiated phenotype and are potentially capable of differentiating into any foetal or adult cell type of any of the three germ layers (endoderm, mesoderm and ectoderm). The population of iPSCs may be clonal i.e. genetically identical cells descended from a single common ancestor cell. iPSCs may express one or more of the following pluripotency associated markers: POU5f1 (Oct4), Sox2, Alkaline Phosphatase, SSEA-3, Nanog, SSEA-4, Tra-1-60, KLF4 and c-myc, preferably one or more of POU5f1, NANOG and SOX2. An iPSC may lack markers associated with specific differentiative fates, such as Bra, Sox17, FoxA2, αFP, Sox1, NCAM, GATA6, GATA4, Hand1 and CDX2. In particular, an iPSC may lack markers associated with endodermal fates.

Preferably, the iPSCs are human IPSCs (hiPSCs).

In some embodiments, iPSCs may be gene edited, for example to inactivate or delete HLA genes or other genes associated with immunogenicity or GVHD.

IPSCs may be derived or reprogrammed from donor cells, which may be somatic cells or other antecedent cells obtained from a source, such as a donor individual. The donor cells may be mammalian, preferably human cells. Suitable donor cells include adult fibroblasts and blood cells, for example peripheral blood cells, such as HPCs or mononuclear cells. Suitable donor cells for reprogramming into iPSCs as described herein may be obtained from a donor individual. In preferred embodiments, the donor individual may be a different person to the patient or recipient individual to whom the immune cells will be administered following production as described herein (allogeneic treatment). For example, the donor individual may be a healthy individual who is human leukocyte antigen (HLA) matched (either before or after donation) with a recipient individual suffering from cancer. In other embodiments, the donor individual may not be HLA matched with the recipient individual. Preferably, the donor individual may be a neonate (new-born), for example the donor cells may be obtained from a sample of umbilical cord blood.

Suitable donor individuals are preferably free of communicable viral (e.g. HIV, HPV, CMV) and adventitious agents (e.g. bacteria, mycoplasma), and free of known genetic abnormalities.

In some embodiments, a population of peripheral blood cells, such as HPCs, for reprogramming may be isolated from a blood sample, preferably an umbilical cord sample, obtained from the donor individual. Suitable methods for the isolation of HPCs and other peripheral blood cells, are well-known in the art and include, for example magnetic activated cell sorting (see, for example, Gaudernack et al 1986 J Immunol Methods 90 179), fluorescent activated cell sorting (FACS: see for example, Rheinherz et al (1979) PNAS 764061), and cell panning (see for example, Lum et al (1982) Cell Immunol 72 122). HPCs may be identified in a sample of blood cells by expression of CD34. In other embodiments, a population of fibroblasts for reprogramming may be isolated from a skin biopsy following dispersal using collagenase or trypsin and out-growth in appropriate cell culture conditions.

Donor cells are typically reprogrammed into iPSCs by the introduction of reprogramming factors, such as Oct4, Sox2 and Klf4 into the cell. The reprogramming factors may be proteins or encoding nucleic acids and may be introduced into the differentiated cells by any suitable technique, including plasmid, transposon or more preferably, viral transfection or direct protein delivery. Other reprogramming factors, for example Klf genes, such as Klf-1,-2,-4 and-5; Myc genes such as C-myc, L-myc and N-myc; Nanog; SV40 Large T antigen; Lin28; and short hairpins (shRNA) targeting genes such as p53, may also be introduced into the cell to increase induction efficiency. Following introduction of the reprogramming factors, the donor cells may be cultured. Cells expressing pluripotency markers may be isolated and/or purified to produce a population of iPSCs. Techniques for the production of iPSCs are well-known in the art (Yamanaka et al Nature 2007;448:313-7; Yamanaka 6 2007 Jun. 7; 1 (1): 39-49; Kim et al Nature. 2008 Jul. 31; 454 (7204): 646-50;Takahashi Cell. 2007 Nov. 30; 131 (5): 861-72. Park et al Nature. 2008 Jan. 10; 451 (7175): 141-6; Kimet et al Cell Stem Cell. 2009 Jun. 5;4 (6): 472-6; Vallier, L., et al. Stem Cells, 2009. 9999 (999A): p. N/A; Baghbaderani et al 2016; Stem Cell Rev. 2016 August; 12 (4): 394-420; Baghbaderani et al. (2015) Stem Cell Reports, 5 (4), 647-659).

Conventional techniques may be employed for the culture and maintenance of iPSCs (Vallier, L. et al Dev. Biol. 275, 403-421 (2004), Cowan, C. A. et al. N. Engl. J. Med. 350, 1353-1356 (2004), Joannides, A. et al. Stem Cells 24, 230-235 (2006) Klimanskaya, I. et al. Lancet 365, 1636-1641 (2005), Ludwig, T. E. et al. Nat. Biotechnol. 24, 185-187 (2006)). IPSCs for use in the present methods may be grown in defined conditions or on feeder cells. For example, iPSCs may be conventionally cultured in a culture dish on a layer of feeder cells, such as irradiated mouse embryonic fibroblasts (MEF), at an appropriate density (e.g. 10⁵ to 10⁶ cells/60 mm dish), or on an appropriate substrate, in a feeder conditioned or defined iPSC maintenance medium. iPSCs for use in the present methods may be passaged by enzymatic or mechanical means. In some embodiments, iPSCs may be passaged on matrigel™ or an ECM protein, such as vitronectin, in an iPSC maintenance medium, such as mTeSR™1 or TeSR™2 (StemCell Technologies) or E8 flex (Life Thermo) culture medium.

The IPSCs may be transfected with a nucleic acid comprising the heterologous expression cassette, such that the cassette is integrated into the genome of the IPSC. Suitable techniques are well-established in the art. Transfection at the iPSC stage allows the isolation of a single clone and the differentiation of a homogeneous cell population.

Nucleic acid may be introduced into the cells by any convenient technique. Suitable techniques for transporting the heterologous expression cassette into the iPSCs are well known in the art and include calcium phosphate transfection, DEAE-Dextran, electroporation, liposome-mediated transfection, by gene editing into a specific location gene editing, for example AAV mediated gene editing, and transduction using retrovirus or other virus, e.g. vaccinia or lentivirus. In some embodiments, a CAS9 guide RNA ribonucleoprotein complex (RNP) may be delivered by electroporation and a nucleic acid molecule, for example a DNA molecule, such as targeting vector, encoding the expression cassette would be packaged as an rAAV (serotype 6). Alternatively, the RNA and DNA molecule encoding the expression cassette (as ssDNA) could co delivered by electroporation.

Targeting to a site of integration in the genome of the iPSC is provided by the combination of the use of CRISPR/Cas9 to generate double strand breaks and the homology arms within the nucleic molecule. Suitable sites of integration include gene loci and safe harbour loci and are described in more detail above. All clones may be screened to confirm integration at the correct site.

When introducing or incorporating a heterologous nucleic acid into an iPSC, certain considerations must be taken into account. The nucleic acid to be inserted should be assembled within a construct or vector which contains effective regulatory elements which will drive transcription in the T cell. Many known techniques and protocols for manipulation and transformation of nucleic acid, for example in preparation of nucleic acid constructs, introduction of DNA into cells and gene expression are described in detail in Protocols in Molecular Biology, Second Edition, Ausubel et al. eds. John Wiley & Sons, 1992. In some embodiments, nucleic acid may be introduced into the cells by gene editing. For example, a DNA double strand break (DSB) at a target site may be induced by a CRISPR/Cas9 system and the repair of the DSB may introduce the heterologous nucleic acid into the cell genome at the target site or the nucleic acid may be introducing using an rAAV vector (AAV mediated gene editing; Hirsch et al 2014 Methods Mol Biol 1114 291-307).

Also provided is an IPSC comprising a heterologous expression cassette integrated into the genome thereof,
wherein the expression cassette comprises:
   (a) a coding sequence for a production T cell receptor (TCR),
   (b) a constitutive promoter operably linked to the coding sequence, and
   (c) a targeting site.

The targeting site may be a 5′ targeting site and the cassette may further comprise a 3′ targeting site.

Heterologous expression cassettes are described in more detail above.

The iPSCs may be differentiated and matured into immune cells, such as T cells, in a series of steps. Differentiation and maturation of the cell populations in these steps is induced by culturing the cells in a culture medium supplemented with a set of differentiation factors. The set of differentiation factors for each culture medium is preferably exhaustive and medium may be devoid of other differentiation factors. In preferred embodiments, the culture media are chemically defined media. For example, a culture medium may consist of a chemically defined nutrient medium that is supplemented with an effective amount of one or more differentiation factors, as described below. A chemically defined nutrient medium may comprise a basal medium that is supplemented with one or more serum-free culture medium supplements.

Differentiation factors are factors which modulate, for example promote or inhibit, a signalling pathway which mediates differentiation in a mammalian cell. Differentiation factors may include growth factors, cytokines and small molecules which modulate one or more of the Activin/Nodal, FGF, Wnt or BMP or signalling pathways thereof. Examples of differentiation factors include Activin/Nodal, FGFs, BMPs, retinoic acid, vascular endothelial growth factor (VEGF), stem cell factor (SCF), TGFβ ligands, GDFs, LIF, Interleukins, GSK-3 inhibitors and phosphatidylinositol 3-kinase (PI3K) inhibitors.

Differentiation factors which are used in one or more of the media described herein include TGFβ ligands, such as activin, fibroblast growth factor (FGF), bone morphogenetic protein (BMP), stem cell factor (SCF), vascular endothelial growth factor (VEGF), GSK-3 inhibitors (such as CHIR-99021), interleukins, and hormones, such as IGF-1 and angiotensin II. A differentiation factor may be present in a medium described herein in an amount that is effective to modulate a signalling pathway in cells cultured in the medium.

In some embodiments, a differentiation factor listed above or below may be replaced in a culture medium by a factor that has the same effect (i.e. stimulation or inhibition) on the same signalling pathway. Suitable factors are known in the art and include proteins, nucleic acids, antibodies and small molecules.

The extent of differentiation of the cell population during each step may be determined by monitoring and/or detecting the expression of one or more cell markers in the population of differentiating cells. For example, an increase in the expression of markers characteristic of the more differentiated cell type or a decrease in the expression of markers characteristic of the less differentiated cell type may be determined. The expression of cell markers may be determined by any suitable technique, including immunocytochemistry, immunofluorescence, RT-PCR, immunoblotting, fluorescence activated cell sorting (FACS), and enzymatic analysis. In preferred embodiments, a cell may be said to express a marker if the marker is detectable on the cell surface. For example, a cell which is stated herein not to express a marker may display active transcription and intracellular expression of the marker gene but detectable levels of the marker may not be present on the surface of the cell.

A population of partially differentiated cells, for example mesoderm cells, haemogenic endothelium (HE; i.e. haemogenic endothelial cells or HECs), HPCs, or T cell progenitors, that is produced by a step in the methods described herein may be cultured, maintained or expanded before the next differentiation step. Partially differentiated cells may be expanded by any convenient technique.

After each step, the population of partially differentiated cells which is produced by that step may be free or substantially free from other cell types. For example, the population may contain 60% or more, 70% or more, 80% or more or 90% or more partially differentiated cells, following culture in the medium. Preferably, the population of cells is sufficiently free of other cell types that no purification is required. If required, the population of partially differentiated cells may be purified by any convenient technique, such as MACs or FACS.

Cells may be cultured in a monolayer, in the absence of feeder cells, on a surface or substrate coated with extracellular matrix protein, such as fibronectin, laminin or collagen. Suitable techniques for cell culture are well-known in the art (see, for example, Basic Cell Culture Protocols, C. Helgason, Humana Press Inc. U.S. (15 Oct. 2004) ISBN: 1588295451; Human Cell Culture Protocols (Methods in Molecular Medicine S.) Humana Press Inc., U.S. (9 Dec. 2004) ISBN: 1588292223; Culture of Animal Cells: A Manual of Basic Technique, R. Freshney, John Wiley & Sons Inc (2 Aug. 2005) ISBN: 0471453293, Ho W Y et al J Immunol Methods. (2006) 310:40-52, Handbook of Stem Cells (ed. R. Lanza) ISBN: 0124366430) Basic Cell Culture Protocols' by J. Pollard and J. M. Walker (1997), ‘Mammalian Cell Culture: Essential Techniques’ by A. Doyle and J. B. Griffiths (1997), ‘Human Embryonic Stem Cells’ by A. Chiu and M. Rao (2003), Stem Cells: From Bench to Bedside' by A. Bongso (2005), Peterson & Loring (2012) Human Stem Cell Manual: A Laboratory Guide Academic Press and ‘Human Embryonic Stem Cell Protocols’ by K. Turksen (2006). Media and ingredients thereof may be obtained from commercial sources (e.g. Gibco, Roche, Sigma, Europa bioproducts, R&D Systems). Standard mammalian cell culture conditions may be employed for the above culture steps, for example 37° C., 5% or 21% Oxygen, 5% Carbon Dioxide. Media is preferably changed every two days and cells allowed to settle by gravity.

Cells may be cultured in a culture vessel. Suitable cell culture vessels are well-known in the art and include culture plates, dishes, flasks, bioreactors, and multi-well plates, for example 6-well, 12-well or 96-well plates. The culture vessels are preferably treated for tissue culture, for example by coating one or more surfaces of the vessel with an extracellular matrix protein, such as fibronectin, laminin or collagen. Culture vessels may be treated for tissue culture using standard techniques, for example by incubating with a coating solution as described herein or may be obtained pre-treated from commercial suppliers.

iPSCs may be differentiated into immune cells using a multi-step process that comprises;
(i) differentiating the iPSCs into mesoderm cells,
(ii) differentiating the mesoderm cells into haemogenic endothelial cells (HECs),
(iii) differentiating the HECs into a population of haematopoietic progenitor cells (HPCs),
(iv) differentiating the HPCs into immune cell progenitors; and
(v) maturing the population of progenitor immune cells to produce a population of immune cells that express the heterologous expression cassette.

In a first stage, the population of iPSCs may be differentiated into mesoderm cells. iPSCs may for example be differentiated into mesoderm cells by culturing the population of iPSCs under suitable conditions to promote mesodermal differentiation. For example, the iPSCs cells may be cultured sequentially in first, second and third mesoderm induction media to induce differentiation into mesoderm cells. In preferred embodiments, the first, second and third mesoderm induction media are chemically defined media. For example, the first mesoderm induction medium may consist of a chemically defined nutrient medium supplemented with an effective amount of activin, preferably activin A, for example 50 ng/ml activin A; the second mesoderm induction medium may consist of a chemically defined nutrient medium supplemented with an effective amount of activin preferably activin A, for example 5 ng/ml activin A, BMP, preferably BMP4, for example 10 ng/ml BMP4; and FGF, preferably bFGF (FGF2), for example 5 ng/ml bFGF; and the third mesoderm induction medium may consist of a chemically defined nutrient medium supplemented with an effective amount of activin preferably activin A, for example 5 ng/ml activin A, BMP, preferably BMP4, for example 10 ng/ml BMP4; FGF, preferably bFGF (FGF2), for example 5 ng/ml bFGF; and GSK3 inhibitor, preferably CHIR-99021, for example 10 μM CHIR-99021.

In a second stage, the mesoderm cells may be differentiated into haemogenic endothelial cells. Mesoderm cells may be differentiated into haemogenic endothelial (HE) cells by culturing the population of mesoderm cells under suitable conditions to promote HE differentiation. For example, the mesoderm cells may be cultured in an HE induction medium. In preferred embodiments, the HE induction medium is a chemically defined medium. For example, the HE induction medium may consist of a chemically defined nutrient medium supplemented with effective amounts of VEGF, for example 15 ng/ml VEGF; and SCF, for example 100 ng/ml SCF. Preferably, mesoderm cells are cultured in an HE induction medium consisting of a chemically defined nutrient medium and two differentiation factors, wherein the two differentiation factors are SCF and VEGF.

In a third stage, haemogenic endothelial cells may be differentiated into haematopoietic progenitor cells (HPCs). Haemogenic endothelial (HE) cells may be differentiated into haematopoietic progenitor cells (HPCs) by culturing the population of HE cells under suitable conditions to promote haematopoietic differentiation. For example, the HE cells may be cultured in a haematopoietic induction medium. In preferred embodiments, the haematopoietic induction medium is a chemically defined medium. For example, the haematopoietic induction medium may consist of a chemically defined nutrient medium supplemented with effective amounts of VEGF, for example 15 ng/ml; SCF, for example 100 ng/ml; thrombopoietin (TPO), for example 30 ng/ml; Flt3 ligand (FLT3L), for example 25ng./ml; IL-3, for example 25ng/ml; IL-6, for example 10 ng/ml; IL-7, for example 10 ng/ml; IL-11, for example 5 ng/ml; IGF-1, for example 25 ng/ml; BMP, for example BMP4 at 10 ng/ml; FGF, for example bFGF at 5 ng/ml; Sonic hedgehog (SHH), for example 25 ng/ml; erythropoietin (EPO), for example 2 u/ml; angiotensin II, for example 10 μg/ml, and an angiotensin Il type 1receptor (AT₁) antagonist, for example losartan, at 100 μM.

In a fourth stage, the HPCs may be differentiated into immune cell progenitors, such as T cell progenitors. Haematopoietic progenitor cells (HPCs) may be differentiated into progenitor immune cells by culturing the population of HPCs under suitable conditions to promote lymphoid differentiation. For example, the haematopoietic progenitor cells may be cultured in a lymphoid expansion medium. In preferred embodiments, the lymphoid expansion medium is a chemically defined medium. For example, the lymphoid expansion medium may consist of a chemically defined nutrient medium supplemented with effective amounts of the above differentiation factors. Suitable lymphoid expansion media are well-known in the art and include Stemspan™ SFEM II (Cat #9605; StemCell Technologies Inc, CA), with Stemspan™ lymphoid expansion supplement (Cat #9915; StemCell Technologies Inc, CA).

In a fifth stage, the progenitor immune cells may be matured into TCR αβ+ immune cells, such as TCR αβ+ T cells. Progenitor immune cells may be matured into TCR αβ+ immune cells by culturing the population of progenitor immune cells under suitable conditions to promote maturation. For example, the progenitor immune cells may be cultured in a maturation medium. In preferred embodiments, the T cell maturation medium is a chemically defined medium. For example, the T cell maturation medium may consist of a chemically defined nutrient medium supplemented with effective amounts of the above differentiation factors. Suitable T cell maturation media are well-known in the art and include Stemspan™ SFEM II (Cat #9605;StemCell Technologies Inc, CA) with Stemspan™ T cell maturation supplement (Cat #9930; StemCell Technologies Inc, CA) and other media suitable for expansion of PBMCs and CD3+ cells, such as ExCellerate Human T cell expansion medium (R& D Systems, USA). Other suitable T cell maturation media may include a basal medium such as IMDM, supplemented with ITS, albumin and lipids, as described elsewhere herein and further supplemented with effective amounts of the above differentiation factors.

In a sixth stage, the population of TCR αβ+ immune cells, such as TCR αβ+ T cells, may be activated and/or expanded to produce or increase the proportion of single positive CD4+ immune cells, or more preferably single positive CD8+ immune cells. Suitable methods for activating and expanding immune cells, such as T cells, are well-known in the art. For example, T cells may be exposed to a T cell receptor (TCR) agonist under appropriate culture conditions. Suitable TCR agonists include ligands, such as peptides displayed on a class I or II MHC molecules (MHC-peptide complexes) on the surface of a bead or an antigen presenting cell, such as a dendritic cell, and soluble factors, such as anti-TCR antibodies, for example anti-CD28 antibodies, and multimeric MHC-peptide complexes, such as MHC-peptide tetramers, pentamers or dextramers.

Suitable conditions and media for use in stages 1 to 6 are known in the art. Some preferred conditions and media are disclosed in WO2021/032836, WO2021/032855, WO2021/032851 and WO2021/032852, the contents of which, including conditions and culture media, are incorporated by reference.

Directed differentiated and maturation as described above generates immune cells comprising a heterologous expression cassette. Also provided is an immune cell comprising a heterologous expression cassette integrated into the genome thereof,
wherein the expression cassette comprises;
   (a) a coding sequence for a production T cell receptor (TCR),
   (b) a constitutive promoter operably linked to the coding sequence, and
   (c) a targeting site.

The targeting site may be a 5′ targeting site and the cassette may further comprise a 3′ targeting site.

Immune cells are described elsewhere herein. For example, the immune cell may be an TCR αβ+ immune cell, such as an TCR αβ+ T cell.

Following production, immune cells comprising the heterologous expression cassette may be stored or primed for use in therapy, such as adoptive cellular therapy or adoptive immunotherapy. The immune cells are primed by introducing an expression construct encoding a therapeutic TCR into the cell genome at the site of the heterologous expression cassette as described herein. For example the immune cells may be primed by replacing the heterologous expression cassette with an expression construct encoding a therapeutic TCR as described herein. The primed population of immune cells that express the therapeutic TCR may be for use as a medicament. For example, a population of immune cells that express the therapeutic TCR may be used in immunotherapy, for example adoptive cellular therapy or adoptive immunotherapy.

Adoptive cellular therapy or adoptive immunotherapy refers to the adoptive transfer of human immune cells, such as T lymphocytes, that express TCRs that are specific for antigens or peptides thereof expressed on target cells in a patient and/or TCRs that are specific for peptide MHC complexes expressed on the target cells. This can be used to treat a range of diseases depending upon the target chosen, e.g., tumour specific antigens to treat cancer. Adoptive cellular therapy (ACT) involves removing a portion of a donor's cells, for example, white blood cells. The cells are then used to generate iPSCs in vitro and these iPSCs are transfected with a heterologous expression cassette and used to efficiently generate immune cells. The immune cells may be expanded, washed, concentrated, and/or then frozen to allow time for testing, shipping and storage until a patient is ready to receive an infusion of immune cells The immune cells are then primed by insertion of an expression construct encoding a therapeutic TCR that is specific for an antigen or peptide thereof expressed on target cells, such as cancer cells, and/or specific for peptide MHC complexes on target cells, such as cancer cells, in the patient at the site of the heterologous expression cassette as described herein. The expression construct may replace the heterologous expression cassette. The nucleotide sequence encoding the therapeutic TCR may be derived from cancer-reactive immune cells, such as tumour infiltrating lymphocytes, obtained from the patient.

In some embodiments, a population of immune cells may be primed by insertion at the site of the heterologous expression cassettes of the immune cells in the population, a population of expression constructs encoding therapeutic TCRs that are specific for different antigens or peptides thereof expressed on target cells, such as cancer cells, and/or specific for different peptide MHC complexes on target cells, such as cancer cells, in the patient as described herein. For example, the heterologous expression cassettes may be replaced by the expression constructs. The therapeutic TCRs encoded by the population of expression constructs may be reactive with different tumour antigens in the patient. The nucleotide sequences encoding the different therapeutic TCRs in the population of expression constructs may be derived from cancer-reactive immune cells, such as tumour infiltrating lymphocytes, obtained from the patient. A population of immune cells primed in this manner may be reactive with multiple different tumour antigens in the patient.

After production and priming, a population of immune cells expressing a therapeutic TCR or therapeutic TCRs produced as described herein may be admixed with other reagents, such as buffers, carriers, diluents, preservatives and/or pharmaceutically acceptable excipients. Suitable reagents are described in more detail below. A method described herein may comprise admixing the population of immune cells with a pharmaceutically acceptable excipient.

Pharmaceutical compositions suitable for administration (e.g. by infusion), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents. Examples of suitable isotonic vehicles for use in such formulations include Sodium Chloride Injection, Ringer's Solution, or Lactated Ringer's Injection. Suitable vehicles can be found in standard pharmaceutical texts, for example, Remington's Pharmaceutical Sciences, 18th edition, Mack Publishing Company, Easton, Pa., 1990.

In some preferred embodiments, the immune cells may be formulated into a pharmaceutical composition suitable for intravenous infusion into an individual.

The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Other aspects of the invention provide the use of a population of immune cells that express the therapeutic TCR or the therapeutic TCRs produced as described herein for the manufacture of a medicament for the treatment of cancer, a population of immune cells that express the therapeutic TCR or the therapeutic TCRs produced as described herein for the treatment of cancer, and a method of treatment of cancer comprising administering a population of immune cells that express the therapeutic TCR or the therapeutic TCRs produced as described herein to an individual in need thereof.

The population of immune cells may be allogeneic i.e. the immune cells may be originally obtained from a different individual to the individual to whom they are subsequently administered (i.e. the donor and recipient individual are different). Allogeneic refers to a graft derived from a different animal of the same species.

The donor and recipient individuals may be HLA matched to avoid GVHD and other undesirable immune effects, such as rejection. Alternatively, the donor and recipient individuals may not be HLA matched, or HLA genes in the cells from the donor individual may be modified, for example by gene editing, to remove any HLA mismatch with the recipient.

A suitable population of immune cells for administration to a recipient individual may be produced by a method comprising providing an initial population of cells, preferably T cells, obtained from a donor individual, reprogramming the cells into iPSCs, transfecting the iPSCs with a heterologous expression cassette, differentiating the iPSCs into immune cells, and priming the immune cells by replacing the heterologous expression cassette with an expression construct encoding a therapeutic TCR which binds specifically to cancer cells and/or an antigen or peptide thereof presented by cancer cells optionally in complex with MHC, in the recipient individual; or by replacing the heterologous expression cassette in the immune cells with a population of expression constructs each encoding a therapeutic TCR which binds specifically to a different antigen or peptide thereof presented by cancer cells optionally in complex with MHCs, in the recipient individual.

Following administration of the immune cells expressing the therapeutic TCR or the therapeutic TCRs, the recipient individual may exhibit a cell mediated immune response against cancer cells in the recipient individual. This may have a beneficial effect on the cancer condition in the individual.

As used herein, the terms “cancer,” “neoplasm,” and “tumour” are used interchangeably and, in either the singular or plural form, refer to cells that have undergone a malignant transformation that makes them pathological to the host organism.

Primary cancer cells can be readily distinguished from non-cancerous cells by well-established techniques, particularly histological examination. A cancer cell includes not only a primary cancer cell, but any cell derived from a cancer cell ancestor. This includes metastasized cancer cells, and in vitro cultures and cell lines derived from cancer cells. When referring to a type of cancer that normally manifests as a solid tumour, a “clinically detectable” tumour is one that is detectable on the basis of tumour mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient.

Cancer conditions may be characterised by the abnormal proliferation of malignant cancer cells and may include leukaemias, such as AML, CML, ALL and CLL, lymphomas, such as Hodgkin lymphoma, non-Hodgkin lymphoma and multiple myeloma, and solid cancers such as sarcomas, skin cancer, melanoma, bladder cancer, brain cancer, breast cancer, uterus cancer, ovary cancer, prostate cancer, lung cancer, colorectal cancer, cervical cancer, liver cancer, head and neck cancer, oesophageal cancer, pancreas cancer, renal cancer, adrenal cancer, stomach cancer, testicular cancer, cancer of the gall bladder and biliary tracts, thyroid cancer, thymus cancer, cancer of bone, and cerebral cancer, as well as cancer of unknown primary (CUP).

Cancer cells within an individual may be immunologically distinct from normal somatic cells in the individual (i.e. the cancerous tumour may be immunogenic). For example, the cancer cells may be capable of eliciting a systemic immune response in the individual against one or more antigens expressed by the cancer cells. The tumour antigens that elicit the immune response may be specific to cancer cells or may be shared by one or more normal cells in the individual.

The cancer cells of an individual suitable for treatment as described herein may express the antigen and/or may be of correct HLA type to bind the αβ TCR expressed by the T cells.

An individual suitable for treatment as described above may be a mammal. In preferred embodiments, the individual is a human. In other preferred embodiments, non-human mammals, especially mammals that are conventionally used as models for demonstrating therapeutic efficacy in humans (e.g. murine, primate, porcine, canine, or rabbit animals) may be employed.

In some embodiments, the individual may have minimal residual disease (MRD) after an initial cancer treatment.

An individual with cancer may display at least one identifiable sign, symptom, or laboratory finding that is sufficient to make a diagnosis of cancer in accordance with clinical standards known in the art. Examples of such clinical standards can be found in textbooks of medicine such as Harrison's Principles of Internal Medicine, 15th Ed., Fauci A S et al., eds., McGraw-Hill, New York, 2001. In some instances, a diagnosis of a cancer in an individual may include identification of a particular cell type (e.g. a cancer cell) in a sample of a body fluid or tissue obtained from the individual.

An anti-tumour effect is a biological effect which can be manifested by a reduction in the rate of tumour growth, decrease in tumour volume, a decrease in the number of tumour cells, a decrease in the number of metastases, an increase in life expectancy, or amelioration of various physiological symptoms associated with the cancerous condition. An “anti-tumour effect” can also be manifested by the ability of the peptides, polynucleotides, cells, particularly T cells produced according to the methods of the present invention, and antibodies described herein in prevention of the occurrence of tumour in the first place.

Treatment may be any treatment and/or therapy, whether of a human or an animal (e.g. in veterinary applications), in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, delaying, abating or arresting one or more symptoms and/or signs of the condition or prolonging survival of a subject or patient beyond that expected in the absence of treatment.

Treatment may also be prophylactic (i.e. prophylaxis). For example, an individual susceptible to or at risk of the occurrence or re-occurrence of cancer may be treated as described herein. Such treatment may prevent or delay the occurrence or re-occurrence of cancer in the individual.

In particular, treatment may include inhibiting cancer growth, including complete cancer remission, and/or inhibiting cancer metastasis. Cancer growth generally refers to any one of a number of indices that indicate change within the cancer to a more developed form. Thus, indices for measuring an inhibition of cancer growth include a decrease in cancer cell survival, a decrease in tumour volume or morphology (for example, as determined using computed tomographic (CT), sonography, or other imaging method), a delayed tumour growth, a destruction of tumour vasculature, improved performance in delayed hypersensitivity skin test, an increase in the activity of T cells, and a decrease in levels of tumour-specific antigens. Administration of immune cells modified as described herein may improve the capacity of the individual to resist cancer growth, in particular growth of a cancer already present in the subject and/or decrease the propensity for cancer growth in the individual.

The immune cells or the pharmaceutical composition comprising the immune cells may be administered to a subject by any convenient route of administration, whether systemically/peripherally or at the site of desired action, including but not limited to; parenteral, for example, by infusion. Infusion involves the administration of the T cells in a suitable composition through a needle or catheter. Typically, T cells are infused intravenously or subcutaneously, although the T cells may be infused via other non-oral routes, such as intramuscular injections and epidural routes. Suitable infusion techniques are known in the art and commonly used in therapy (see, e.g., Rosenberg et al., New Eng. J. of Med., 319:1676, 1988).

Typically, the number of cells administered is from about 10⁵ to about 10¹⁰ per Kg body weight, for example any of about 1, 2, 3, 4, 5, 6, 7, 8, or 9, ×10⁵, ×10⁶, ×10⁷, ×10⁸, ×10⁹, or ×10¹⁰ cells per individual, typically 2×10⁸ to 2×10¹⁰ cells per individual, typically over the course of 30 minutes, with treatment repeated as necessary, for example at intervals of days to weeks. It will be appreciated that appropriate dosages of the TCR αβ+ T cells, and compositions comprising the immune cells can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular cells, cytokine release syndrome (CRS), the route of administration, the time of administration, the rate of loss or inactivation of the cells, the duration of the treatment, other drugs, compounds, and/or materials used in combination, and the age, sex, weight, condition, general health, and prior medical history of the patient. The amount of cells and the route of administration will ultimately be at the discretion of the physician, although generally the dosage will be to achieve local concentrations at the site of action which achieve the desired effect without causing substantial harmful or deleterious side-effects.

While the immune cells may be administered alone, in some circumstances the immune cells may be administered cells in combination with the target antigen, APCs displaying the target antigen, CD3/CD28 beads, IL-2, IL7 and/or IL15 to promote expansion in vivo of the population of immune cells. Administration in combination may be by separate, simultaneous or sequential administration of the combined components.

The population of immune cells may be administered in combination with one or more other therapies, such as cytokines e.g. IL-2, CD4+ CD8+ chemotherapy, radiation and immuno-oncology agents, including checkpoint inhibitors, such as anti-B7-H3, anti-B7-H4, anti-TIM3, anti-KIR, anti-LAG3, anti-PD-1, anti-PD-L1, and anti-CTLA4 antibodies. Administration in combination may be by separate, simultaneous or sequential administration of the combined components.

The one or more other therapies may be administered by any convenient means, preferably at a site which is separate from the site of administration of the immune cells.

Administration of immune cells can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician. Preferably, the immune cells are administered in a single transfusion, for example of any of 500 million, 1 billion, 2, 3, 4, 5, 6, 7, 8, 9, 10, 11, 12, 13, 14, 15 billion T cells for example at least 1×10⁹ T cells.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

### Experimental

### IPSC Cell Culture

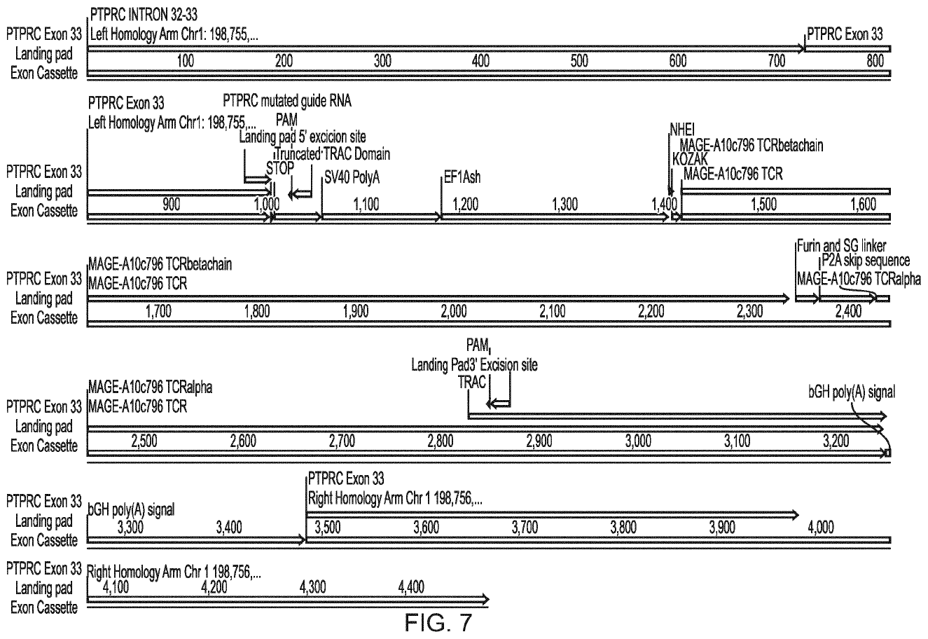

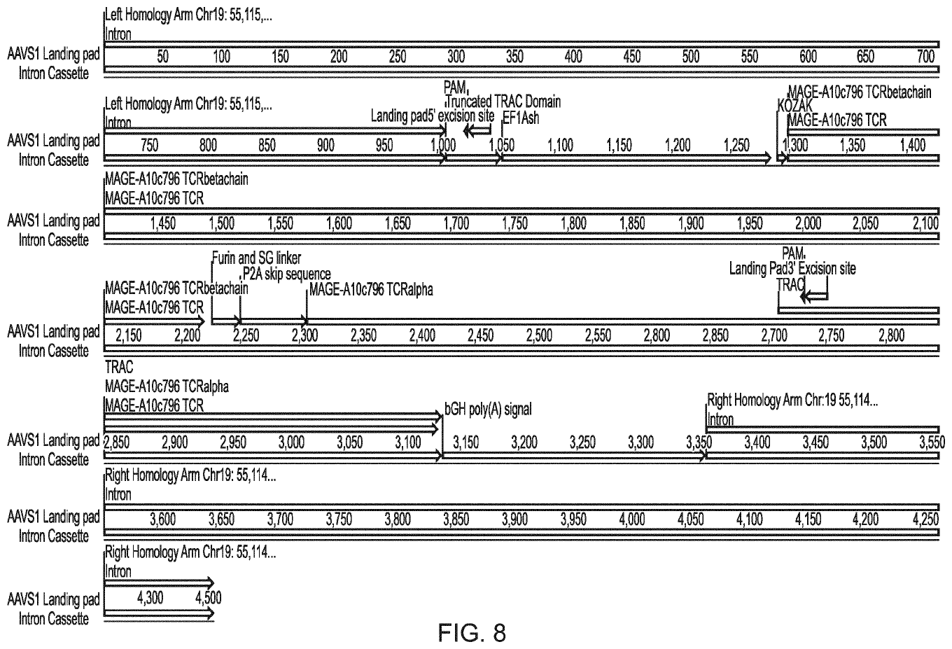

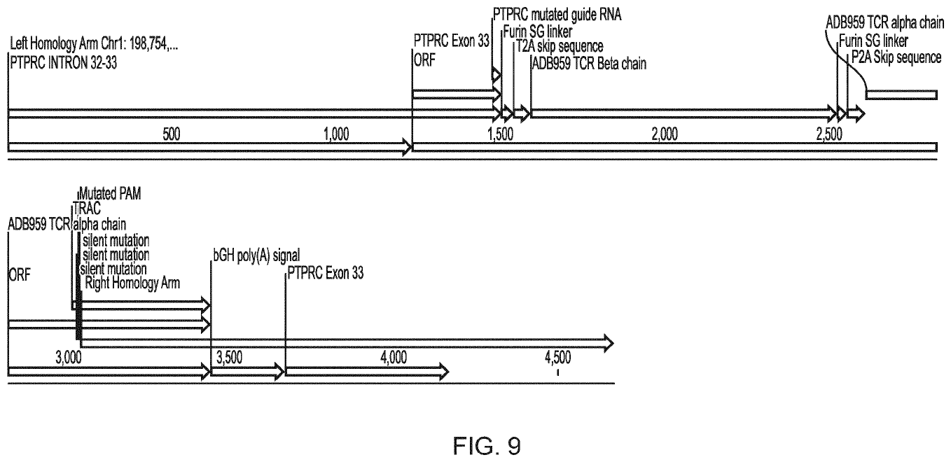

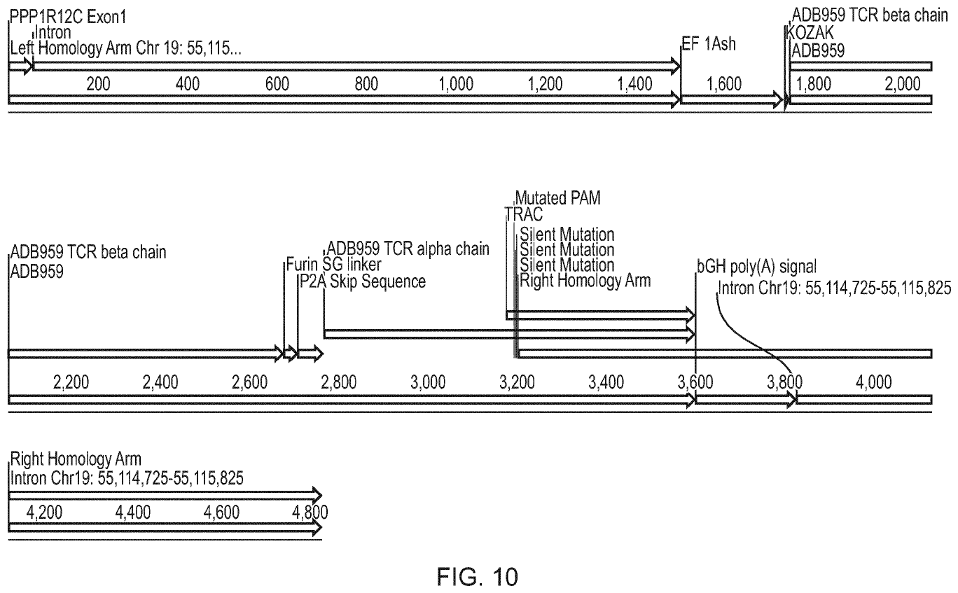

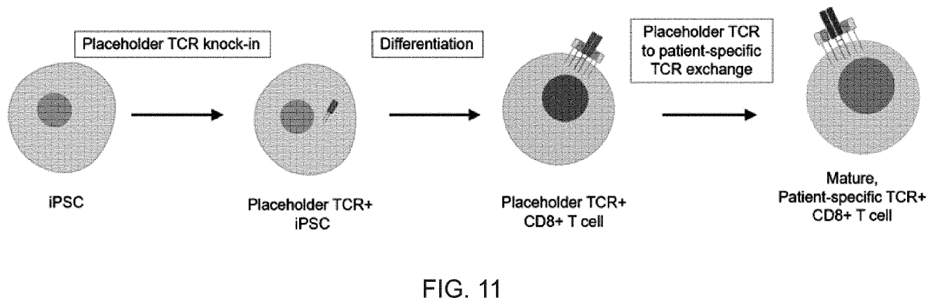

Knock-in of the TCR landing pad constructs (FIGS. 7 and 8) were performed in the iPSC line GR1.1 (Baghbaderani et al 2015; supra. For editing experiments the GR1.1 iPSC line was maintained on tissue-culture treated plates coated with Matriclone (0.25 μg/cm2) (Solemtim) with complete mTeSR™ Plus culture media (STEMCELL Technologies). The GR1.1 iPSC line cell was passaged with Versene (ThermoFisher) or Accutase (STEMCELL Technologies) every 4-5 days. iPSC cultures were maintained in a humidified 37° C., 5% O₂, 5% CO₂ incubator. The expression of pluripotency markers (POU5F1, NANOG, TRA-160 and SOX2) and absence of the differentiation marker SSEA-1 was routinely monitored by FACS analysis. Prior to differentiation edited iPSC clones were adapted for growth maintenance on Synthemax Matrrix (Corning). All other culture conditions were identical.

Generation of rAAV Targeting Vectors

AAV targeting constructs (FIGS. 7 and 8) were generating via Gibson assembly. Homology Arm regions were PCR amplified from genomic DNA and the remaining components of the targeting vector were synthesised synthetically. rAAV vector (Serotype 6) was produced via transient transfection of HEK293T cells with the targeting vector and the pDP6 packaging plasmid (Plasmid Factory). HEK293T were transfected with PEI pro at a plasmid:PEI ratio of 1 μg plasmid:1 μl PEI. rAAV was purified using lodixanol gradient ultracentrifugation according to standard protocols (Strobel et al. (2015) Hum Gen Ther Methods 26 (4) 147-157).

### Guide RNA Sequences

PTPRC Exon 33 was targeted with the guide RNA GCAAGTCCAGCTTTAAATCA (SEQ ID NO: 9). (Chr 1 198756152 to 198756171 (Human GRCh38-Ensembl release 104—May 2021), PPP1r12C Intron 1 was targeted with the guide RNA GTCCCCTCCACCCCACAGTG (SEQ ID NO: 7) (Chr19: 55,115,770-55, 115,790 Human GRCh38-Ensembl release 104—May 2021). TRAC Exon 1 was targeted with the guide RNA AGAGTCTCTCAGCTGGTACA (SEQ ID NO: 6) (Chr14 22547530 to 22547549 Human GRCh38-Ensembl release 104—May 2021 Guide RNA sequences were synthesised by IDT.

### Preparation of Ribonucleoprotein (RNP) Complexes

crRNA and tracrRNA were annealed by initial denaturation at 95° C. for 5 min before cooling to room temperature. Equimolar quantities of annealed crRNA/tracrRNA duplexes and Cas9 protein (IDT) were incubated at room temperature for 15 min to generate 10 μM Ribonucleoprotein (RNP) complexes.

### Knock-in of the ADB796 Landing Pad-Targeting PTPRC Exon 33 or PPP1R12C Intron 1

RNP complexes targeting PTPRC Exon 33 or PPP1R12C Intron 1 were introduced into iPSC cells via nucleofection with the 4D-Nucleofector™ using the 16-well Nucleocuvette™ strips (Lonza). 200×103 GR1.1 were resuspended in buffer P3 (Lonza P3 Primary Cell 4D-Nucleofector™) (10×106/ml). 3 μl of RNP complex (10 μM) was added to 20 μl cell suspension. Nucelofection was performed with program CA-137. Following nucleofection cells were immediately seeded into complete mTESR Plus supplemented with 1×CloneR™ (STEMCELL TECHNOLOGIES). AAV transduction (2×103 Vector genomes/cell) was performed 6-8 hrs post cell seeding. Edited GR1.1 cells were subsequently cultured complete mTESR Plus. Cells were expanded for one passage before isolation of iPSC clones derived from single cells and genotyping of edited clones. Single cells were seeded into 96 well plates using the VIPS instrument from Solentim and expanded for 10-14 days. Edited clones were PCR genotyped according to standard protocols using primers corresponding to genomic DNA outside the homology arm regions and within the TCR transgene. Additionally, ADB796TCR landing pad integration into the desired genomic location (PTPRC Exon 33 or PPP1R12C (Intron 1) was confirmed using TLA analysis (Cergentis).

### Exchange of the MAGE-A10 TCR ADB796 for the MAGE-A4/B2 TCR ADB959

Excision of the ADB796 TCR landing pad was performed with RNPs containing the TGTACCAGCTGAGAGACTCT guide RNA. PTPRCWT/ADB796 landing pad or PPP1R12CWT/ADB796 landing pad iPSC cells were differentiated into iT cells. CD4/CD8 double positive iT cells were harvested at the end of differentiation (Stage 5). Nucleofection was performed with P2 Primary Cell 4D-Nucleofector X kit S™ using the 16-well Nucleocuvette™ strips. 1×10⁶ iT cells were resuspended in 20 μl P2. 3 μl of RNP complex (10 μM) was added to 20 μl cell suspension. Nucleofection was performed with program EH100. AAV transduction (5×10³ vector genomes/cell) was performed 6-8 hrs post cell seeding. Cells were cultured for 72 hrs before phenotyping by FACS. Cells were analysed by FACS for the expression of ADB796 and ADB959 by staining with anti Vbeta13.2 (specific for ADB796) anti-TCR Valpha24 (Specific for ADB959).

### Design and Generation of Landing Pad Strategy 1 TCR A2M10 Placeholder Construct (ADB00794_001)

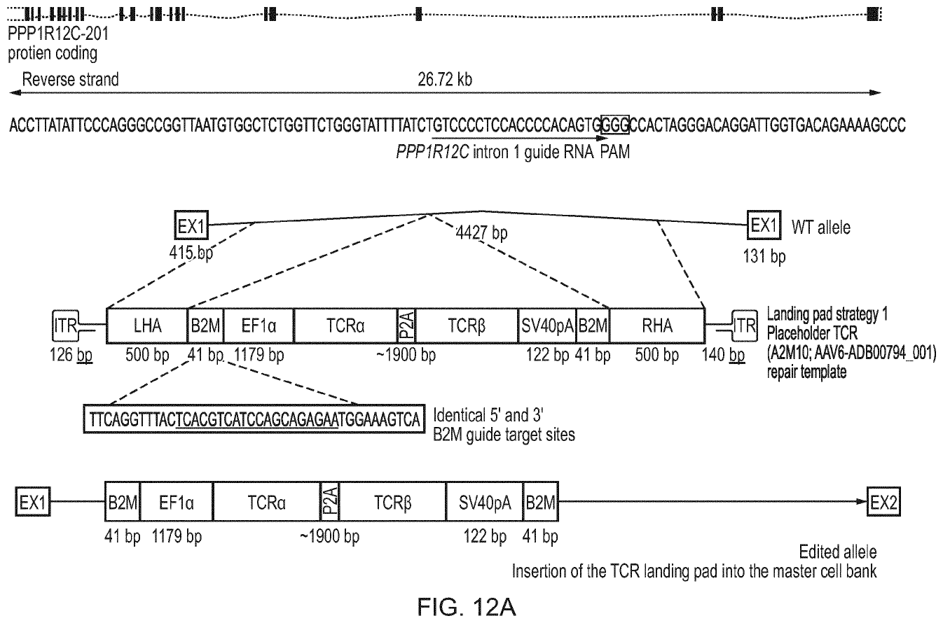

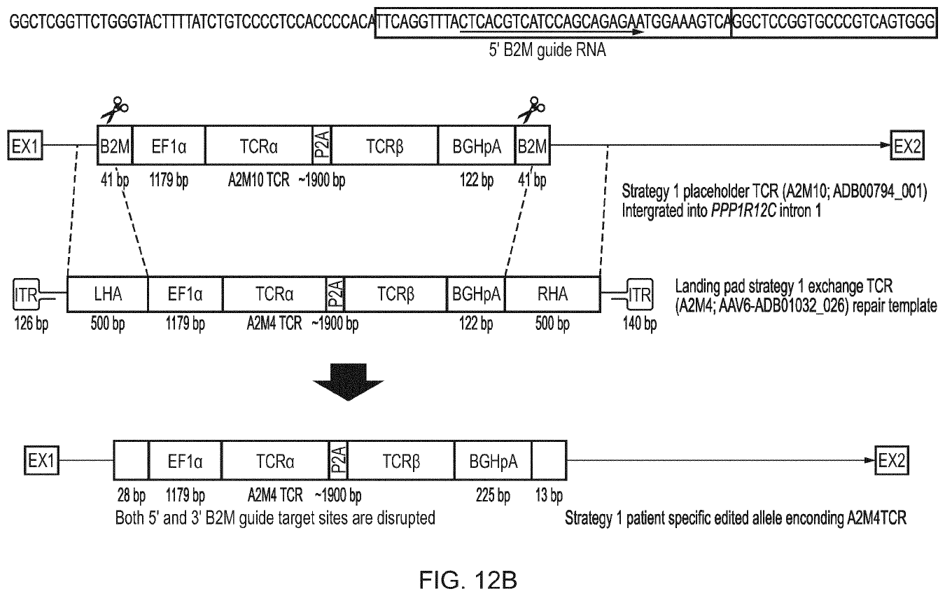

A rAAV repair template encoding recombinant AAV production vector was designed that permitted the constitutive expression of the A2M10 TCR from an EF-1a promoter. The expression cassette was flanked by 41 bp sequences that are present in human B2M. The placeholder cassette was composed of 6 elements as illustrated in FIG. 12:
Right homology arm, required for homology directed repair (HDR)-based integration of the cassette into the PPP1R12C (AAVS1) locus (Present in human, Chr19: 55115773-55115274 GRCh38.p14).
B2M target site present in human B2M (Chr15: 44715435-44715475, GRCh38.p14), which was used as a targetable DNA sequence for the replacement of the placeholder TCR into the exchange TCR (A2M4).
EF-1α promoter.
A2M10 TCR sequence (Border et al., Oncoimmunology, 2018).
SV40 polyadenylation signal
o Left homology arm, required for homology directed repair (HDR)-based integration of the cassette into the PPP1R12C (AAVS1) locus (Present in human PPP1R12C, Chr19: 55116272-55115774, GRCh38.p14).

This construct was designed to be cloned using Gibson cloning into an rAAV production backbone (Agilent pAAV_MCS).

### Generation of Landing Pad Strategy 1 TCR A2M10 Placeholder Construct: ADB00794_001

A2M10 expression cassette was synthesized by Twist Bioscience and inserted into a pTwist-puro backbone. Next, A2M10 expression cassette was PCR amplified using primers containing the 41bp B2M (Chr15: 44715435-44715475, GRCh38.p14) target sequence (SEQ ID NOs: 42 and 43).

Left homology arm (LHA) was amplified from genomic DNA isolated from GR1.1 iPSC (Baghbaderani et al., Stem Cell Reports, 2015) cells using the FWD and REV primers of SEQ ID NOs 44 and 45 (Present in human, Chr19: 55115774-55116274 GRCh38.p14).

Right homology arm (RHA) was amplified from genomic DNA of GR1.1 iPSC cells using the FWD and REV primers of SEQ ID NOs 46 and 47 (Present in human, Chr19: 55115773-55115274 GRCh38.p14):

All PCRs were performed with Q5 DNA polymerase (NEB, M0491L) according to standard protocols. PCR products—A2M10 expression cassette flanked by the B2M target sequences, RHA and LHA were purified by gel extraction using a NucleoSpin Gel and PCR Clean-up Kit (Macherey-Nagel, 740609.50) according to manufacturer's instructions and assembled using the Gibson Assembly® Cloning Kit (NEB, E5510S) into a Notl-digested adeno-associated virus vector backbone (Agilent pAAV-MCS) using equimolar ratio of DNA fragments. Clones were screened by restriction enzyme digest and sequence verified by Sanger sequencing.

### Generation of Landing Pad Strategy 1 TCR A2M4 Exchange Construct: ADB01032_026.

A2M4TCR_BGHpolyA expression plasmid was synthesised by GeneART. A2M4TCR_BGHpolyA (Sanderson et al., Oncoimmunology, 2019) was PCR amplified using the FWD and REV primers of SEQ ID NOs 48 and 49.

Left homology arm (LHA), containing the 500 bp sequence (present in human PPP1R12C, Chr19: 55116273-55115793 GRCh38.p14) was amplified from ADB00794_001 using the FWD and REV primers of SEQ ID NOs 50 and 51.

Right homology arm (RHA), containing the 501 bp sequence (present in human PPP1R12C, Chr19: 55115775-55115274 GRCh38.p14) was amplified from ADB00794_001 using the FWD and REV primers of SEQ ID NOs 52 and 53.

All PCRs were performed with Q5 DNA polymerase (NEB, M0491L) according to standard protocols. PCR products—A2M4TCR_BGHpolyA, RHA and LHA, and EF-1α promoter were purified by gel extraction using a NucleoSpin Gel and PCR Clean-up Kit (Macherey-Nagel, 740609.50) and assembled using the Gibson Assembly® Cloning Kit (NEB, E5510S) into a Notl-digested adeno-associated virus vector backbone (pAAV-MCS) using equimolar ratio of DNA fragments. Clones were screened by restriction enzyme digest and verified by Sanger sequencing. EF-1α promoter was amplified from ADB00794-001 using a forward primer (GGCTCCGGTGCCCGTCAGTGGGC) and reverse primer (GGTGGCGGCAAGCTTGGCAGCGGC).

iPSC Cell Culture

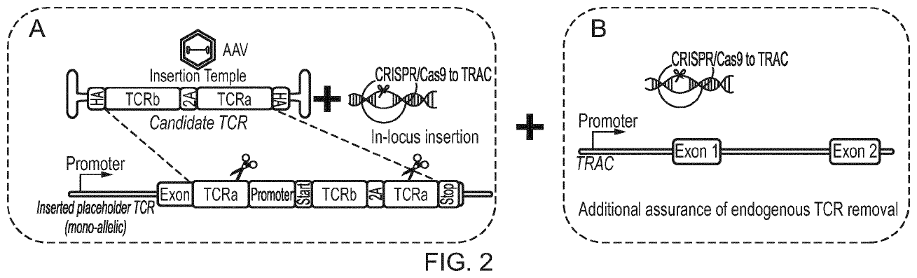

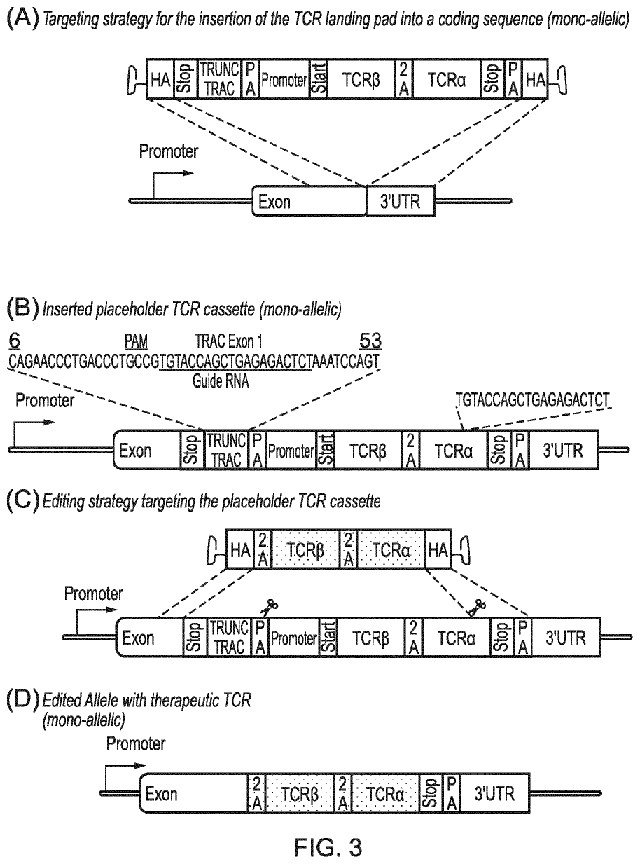

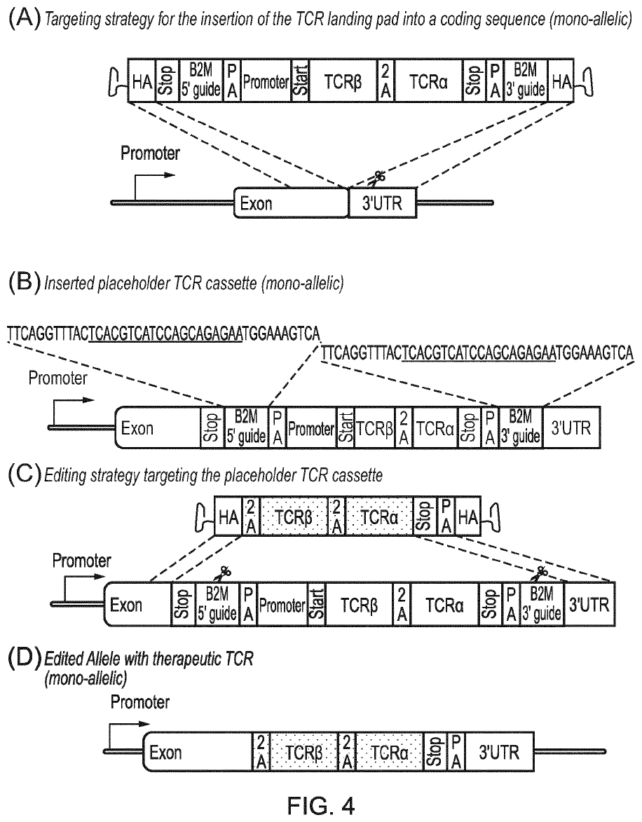

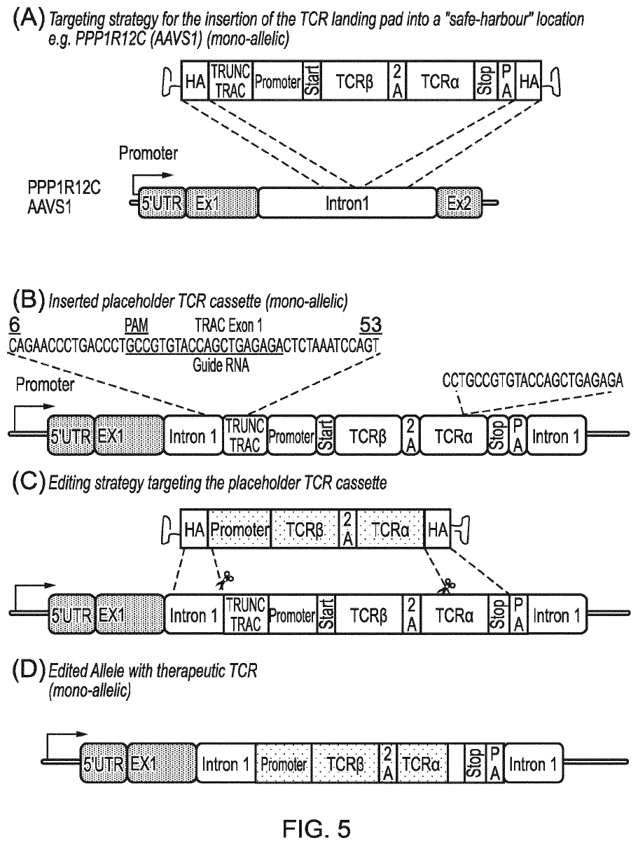

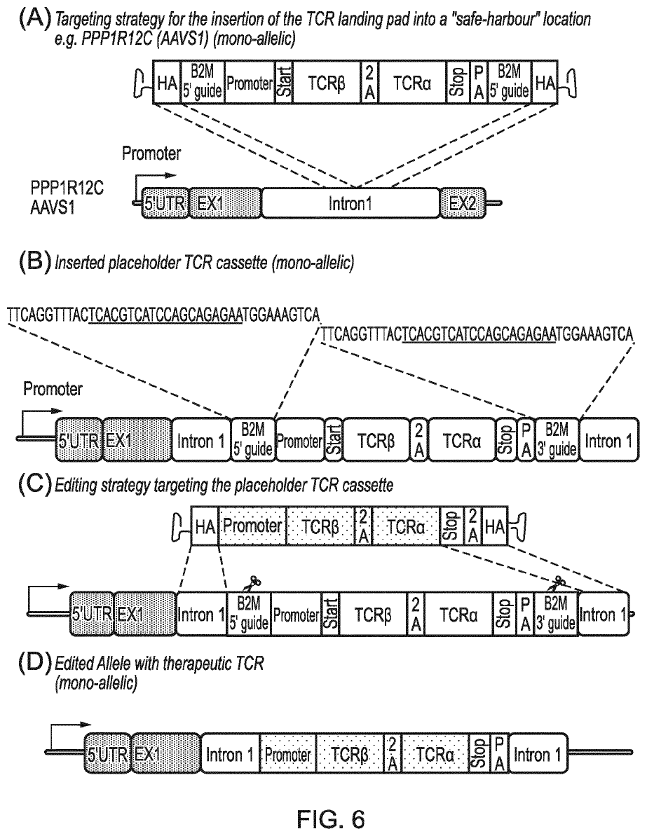

Knock-in of the TCR landing pad construct (FIG. 2) was performed in the iPSC line GR1.1 (Baghbaderani et al 2015). For editing experiments, the GR1.1 iPSC line was maintained on tissue-culture treated plates coated with Vitronectin (0.5 μg/cm2) (Gibco, A14700) with complete mTeSR™ Plus culture media (STEMCELL Technologies, 100-0276). The GR1.1 iPSC line cell was passaged with Versene (ThermoFisher, 15040066) or Accutase (STEMCELL Technologies, 07920) every 4-5 days. iPSC cultures were maintained in a humidified 37° C., 5% O2, 5% CO2 incubator.

Generation of A2M10 Placeholder TCR Knock-in iPSC Cells

Guide RNA targeting intron 1 PPP1R12C locus with sequence GTCCCCTCCACCCCACAGTG (SEQ ID NO: 7; Chr19: 55, 115,770-55, 115,790 Human GRCh38-Ensembl release 104—May 2021) was synthesized by Synthego as a single guide RNA. ADB00794_001 repair template was packaged into AAV6 and purified by Virovek Inc. Purified AAV6-ADB00794_001 virus was used to knock-in the placeholder cassette into GR1.1 iPSC cells using CRISPR-Cas9. Briefly, 250,000 cells were electroporated with 62 pmoles of high fidelity SpyFi Cas9 (Aldevron, 9214-0.25 MG) with 1.2 molar ratio of guide RNA, targeting the PPP12R1C locus using the CA-137 program on a 4D-Nucleofector™ System. Electroporation was performed with P3 Primary Cell 4D-Nucleofector X kit S™ using the 16-well Nucleocuvette™ strips. Following electroporation, the cell suspension was transferred to a 24 well plate containing 500 μl of complete mTESR™ Plus supplemented with 1×CloneR™2 (STEMCELL TECHNOLOGIES, 100-0691) and 1.25×109 AAV6-ADB01032_026 vg added. Cells were then cultured for two weeks, and single cell seeded into 96 well plates using the Solentim Verified In-Situ Plate Seeding (VIPS) platform. Cells were screened for targeted transgene integration using junction PCR (Geisinger, 2016, Nucleic Acids Research). The junction PCR primers of SEQ ID NOs 54-57 were used.

Integration was confirmed both on 5′ and 3′ ends to maximise the confidence in correct gene editing outcomes. Allelic frequencies of the integrated landing pad cassette were confirmed by amplicon PCR followed by agarose gel electrophoresis.

rAAV6-A2M4 Exchange Repair Template Production

rAAV was produced by transient transfection of suspension HEK293T using a two-plasmid system. 2 mM sodium butyrate was included for 24 hours post transfection. Cells were harvested 48 hours post transfection via centrifugation (350 g for 5 minutes), washed with PBS and resuspended in 5 mM Tris pH 8.5, 150 mM NaCl (18 ml/250 ml original culture volume). Resuspended cells were lysed by freeze thaw (frozen on dry ice and thawed in a 37° C. water bath. Cell lysate was treated with Benzonase (250 U/ml, with MgCl2 added to a concentration of 2 mM) at 37° C. for 1 hour. Benzonase treated lysate was clarified by centrifugation (4000×g for 30 minutes) and the supernatant was 0.45 um filtered prior to purification by chromatography on ( ). Clarified lysate was loaded onto a POROS Capture Select AAVX 1 ml column at a flow rate of 0.5 ml/min washed with high salt buffer (10 mM Tris pH 8, 1 M NaCl) and eluted with low pH Glycine buffer (50 mM Glycine pH 2.7, 500 mM NaCl). Eluted AAV was neutralised by addition of Tris pH 8 to a concentration of 80 mM and analysed by SDS-PAGE and dPCR.

### Generation of A2M10 Placeholder-Carrying Progenitor T Cells

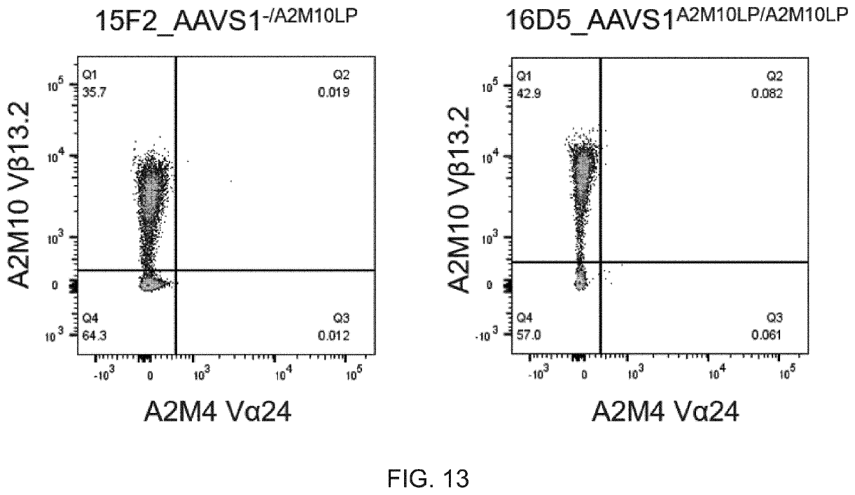

iPSC clones were differentiated into CD34+ hematopoietic progenitor stem cells and then into CD3+iT cell progenitors following in house protocols. Expression of the A2M10 placeholder TCR was confirmed by flow cytometry (FIG. 13).

### A2M10 Placeholder TCR Exchange to A2M4 in Early T Cell Progenitors

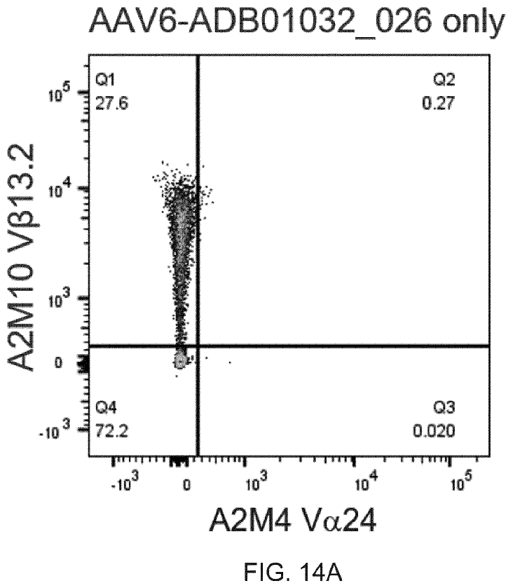

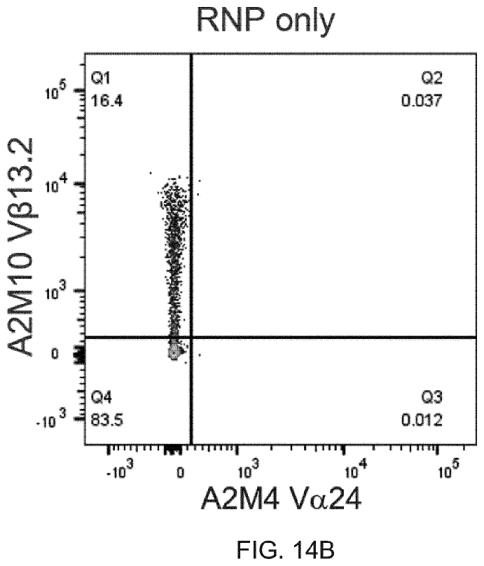

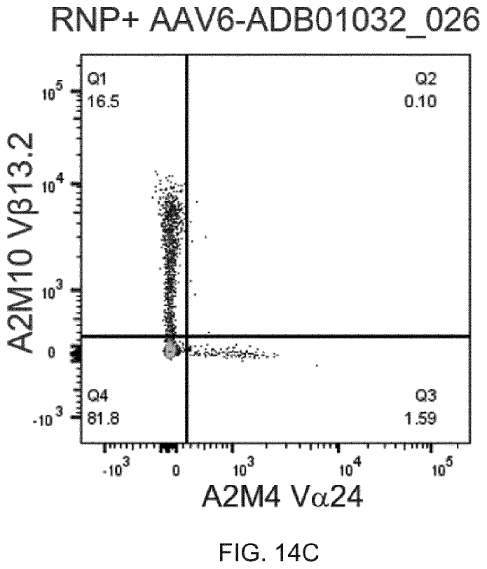

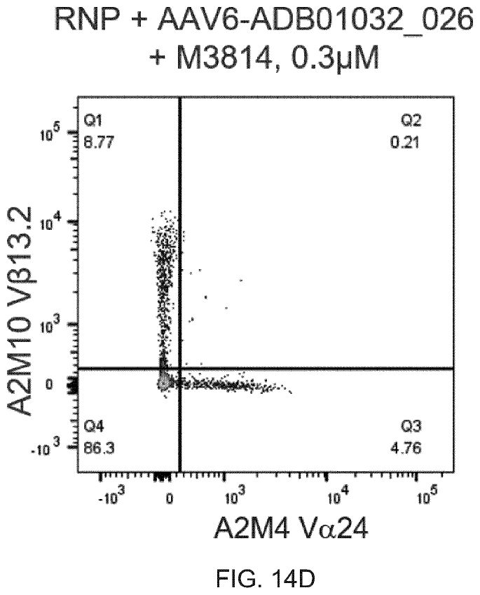

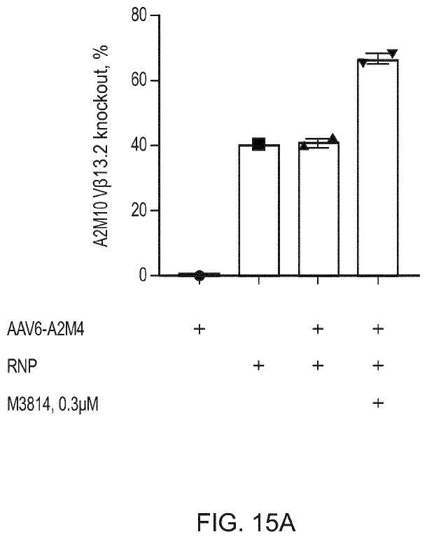

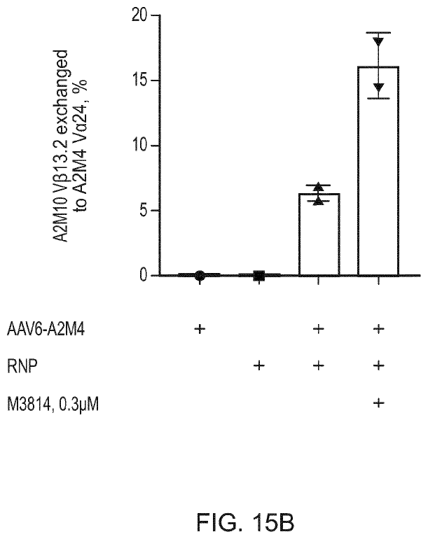

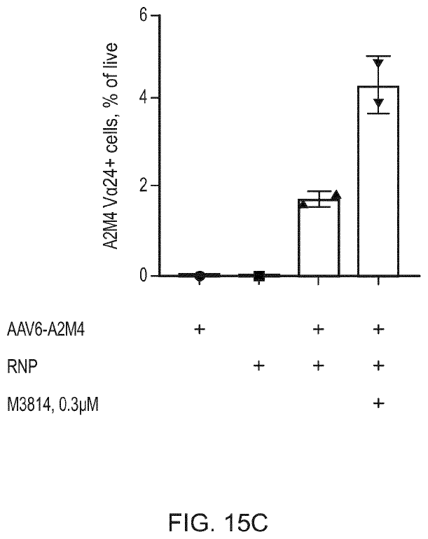

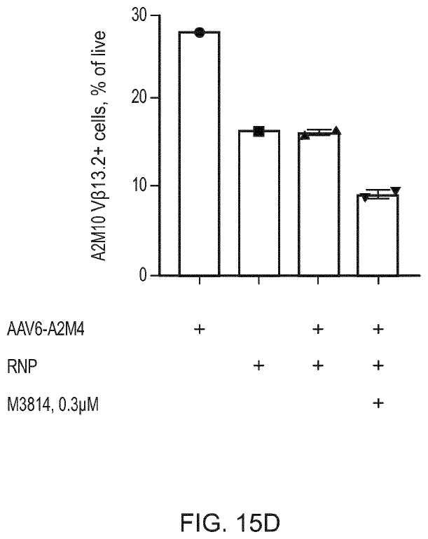

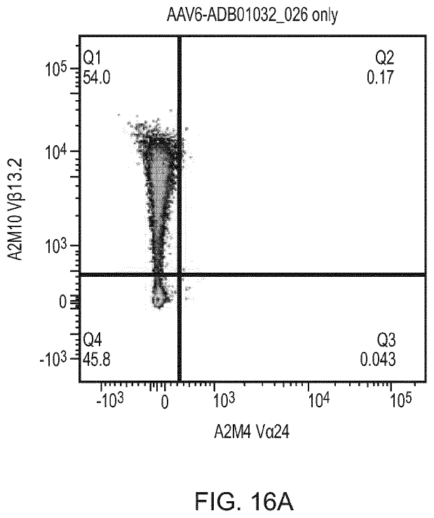

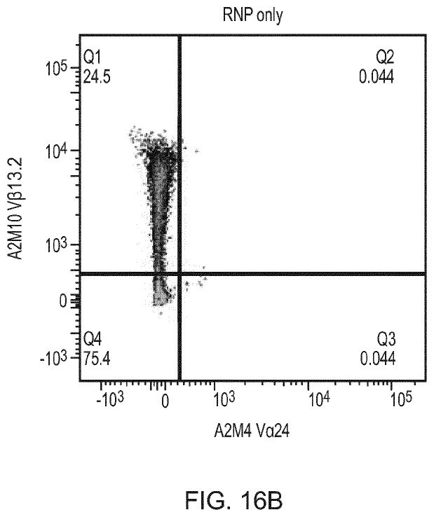

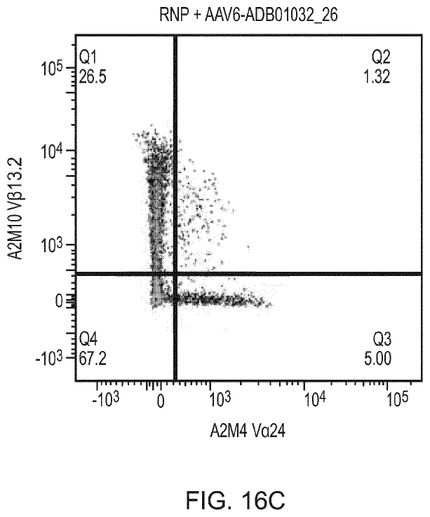

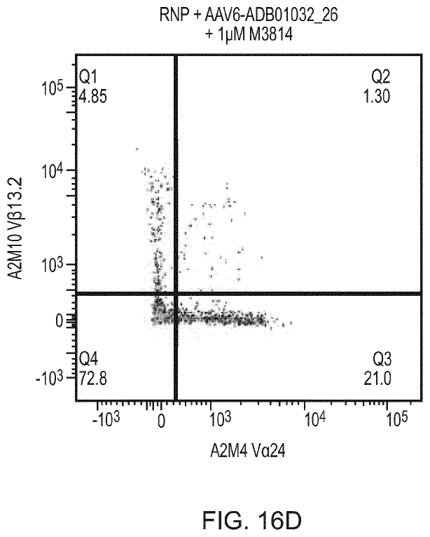

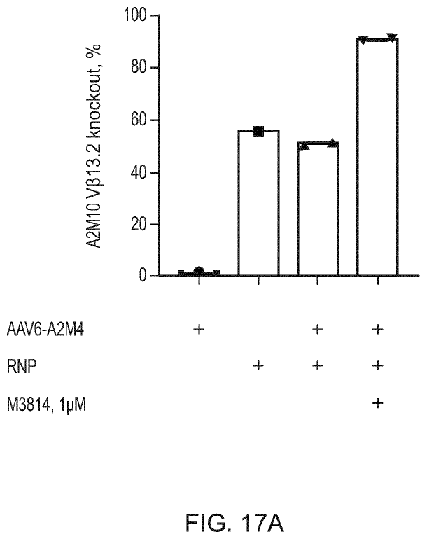

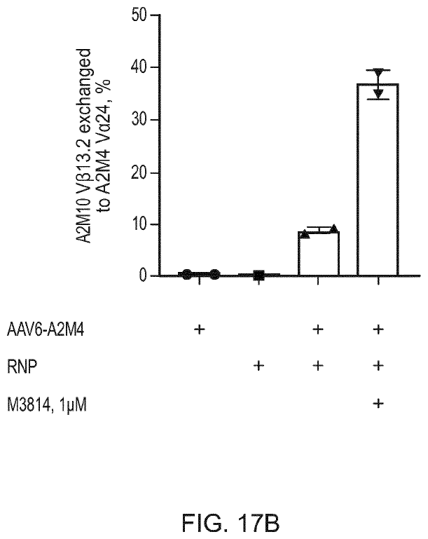

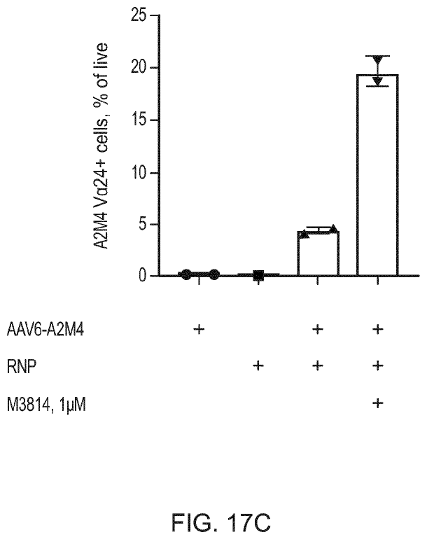

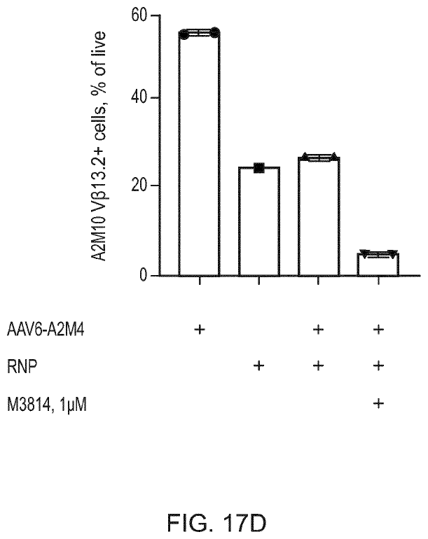

The exchange of the A2M10 TCR for the A2M4 TCR was performed in iT cells differentiated from the iPSC clones 15F2_AAVS1^{-/A2M10LP} and 16D5_AAVS1^{A2M10LP/A2M10LP} and at different stages of differentiation. 15F2_AAVS1^{-/A2M10LP} 3.5×10⁵ iT cell progenitors were electroporated with Cas9-guide RNA ribonucleoprotein (RNP) (SEQ ID NO: 63: ucacgucauccagcagagaa) and transduced with rAAV6-ADB01032_026 immediately following the electroporation (as described in FIG. 12). Media was exchanged 48 h after the transduction, and flow cytometric analysis was performed 24 hours later (FIGS. 14, 15). The list of antibodies used can be found in Table 2. DNA PK inhibitor (M3814, S8586, Selleckchem) was used to improve the HDR editing outcomes (Riesenberg et al., 2019 Nucleic Acids Research, Fu et al., 2021 Nucleic Acids Research). The A2M10 placeholder TCR exchange was reproduced in an independent cell line 16D5_AAVS1^{A2M10LP/A2M10LP} GR1.1 line (FIGS. 16, 17).

### A2M10 Placeholder TCR Exchange to A2M4 in Late-Stage T Cell Progenitors

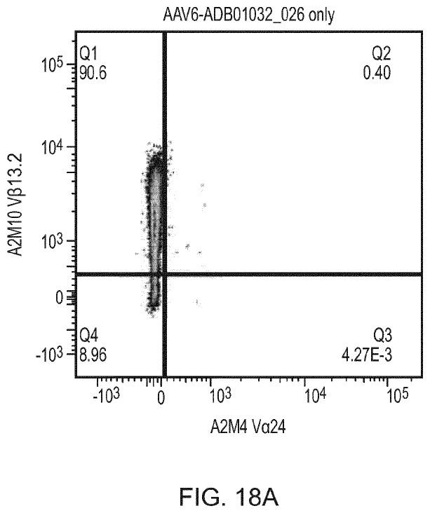

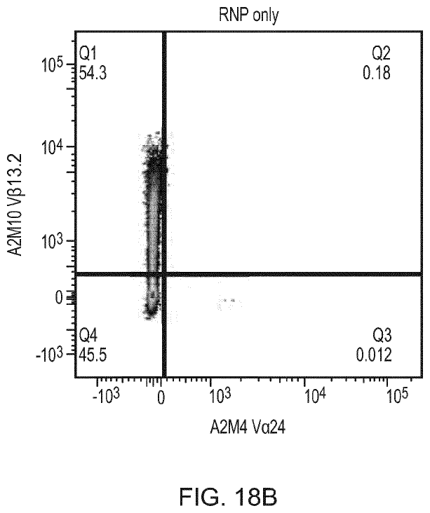

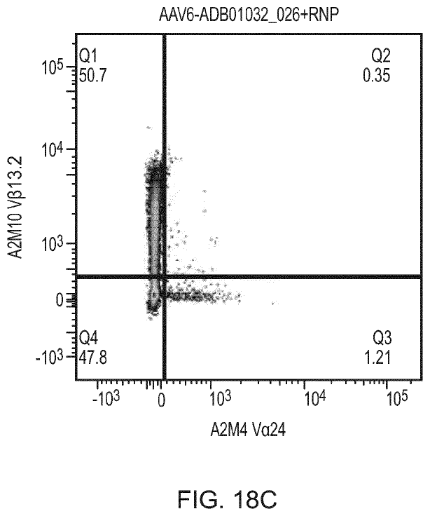

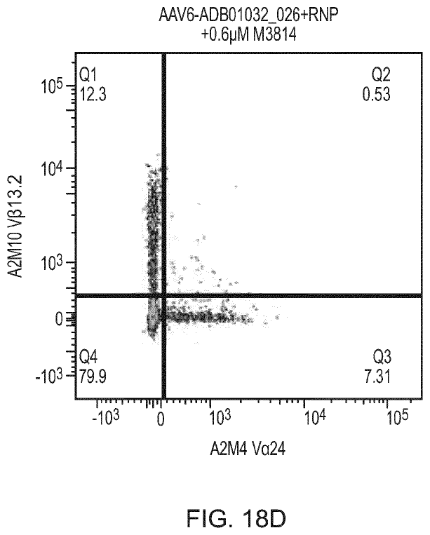

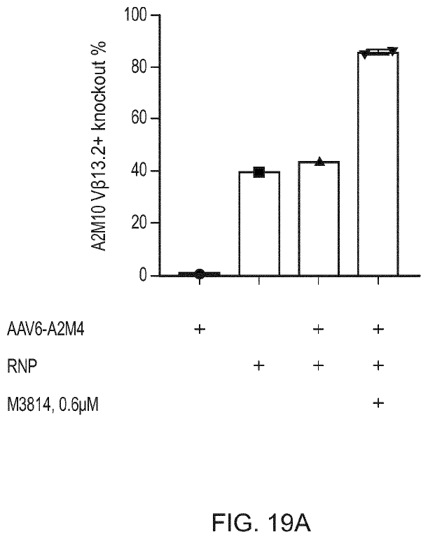

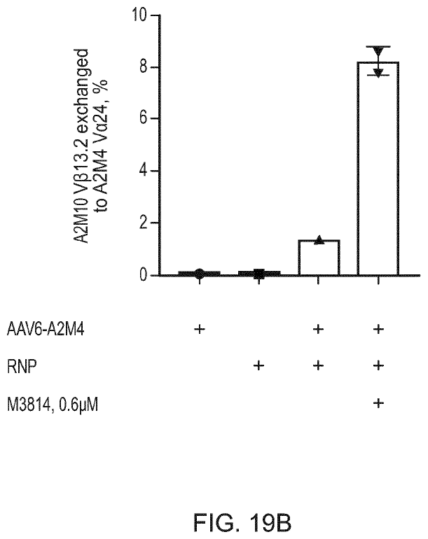

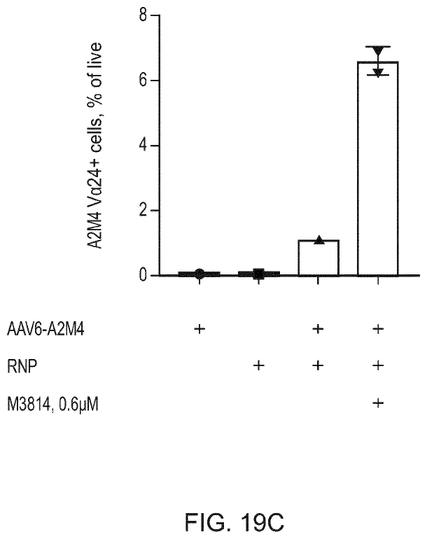

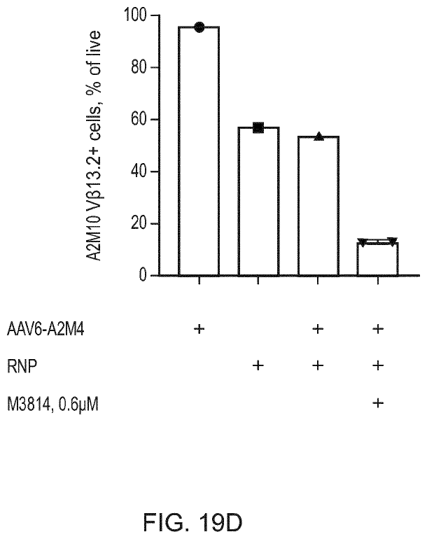

1×10⁶ late-stage 15F2_AAVS1^{-A2M10LP} T cell progenitors were electroporated with Cas9-guide RNA RNP (SEQ ID NO: 63: ucacgucauccagcagagaa) and transduced with AAV6-A2M4 on day 38 following activation with ImmunoCult™ Human CD3/CD28 T Cell Activator (STEMCELL TECHNOLOGIES, 10971) on day 35. Electroporation was performed with P3 Primary Cell 4D-Nucleofector X kit™ using the 96-well Nucleocuvette™ plates with the DZ100 program. Media was exchanged 48 h after transduction, and flow cytometric analysis was performed 48 hours later (FIGS. 18, 19). The list of antibodies used can be found in Table 2.

TABLE 1
safe harbour loci

| PPP1R12C | Chr 19 | Sadelain et al. Nat Rev |
|---|---|---|
| (Intron) | | Cancer 2011 12 (1) 51-8 |
| AAVS1 | | |
| CCR5 | Chr3 | Sadelain et al., 2011 |
| | | supra |
| hROSA26 | Chr 3 | Irion et al. 2007 Nat |
| (THUMPD3) | | Biotech 25 1477-1482 |
| ENVY | Chr12 | Costa et al 2005 Nat |
| | | Meth 2 259-260 |
| GSH1 | Chr 1 195,338,589-195,818,588 | Aznauryan et al 2021 |
| GSH2 | Chr 3 22,720,711-22,761,389 | (bioRxiv) doi.org/ |
| GSH7 | Chr 7 145,090,941-145,219,513 | 10.1101/ |
| GSH8 | Chr 7 145,320,384-145,525,881 | 2021.03.04.433856 |
| GSH31 | Chr X 89,174,426-89,179,074 | |
| SHS231 | Chr 4 58,976,613-632 | Pellenz et al., 2019 Hum |
| | | Gene Ther. 30(7): 814- |
| | | 828 |

TABLE 2
List of antibodies and volumes used to
assess A2M4 and A2M10 TCR expression.

| Antibody | Volume/test(μL) |
|---|---|
| TCR Vβ13.2-PE (BioLegend: 333108) | 2.5 |
| TCR Vα24-PerCP/Cyanine5.5 (BioLegend: 360004) | 2.5 |
| TCRα/β-APC (Miltenyi: 130-113-527) | 2 |
| CD3-FITC (BioLegend: 344810) | 2.5 |
| eBioscience ™ Fixable Viability Dye eFluor ™ 506 | 0.5 |

Sequences

GSGATNFSLLKQAGDVEENP GP
SEQ ID NO: 1-P2A cleavage sequence
GGAAGCGGAGCT ACTAACTTCA GCCTGCTGAA GCAGGCTGGA GACGTGGAGG AGAACCCTGG GCCT
SEQ ID NO: 2-nucleotide sequence encoding P2A peptide.
GCTCCGGTGCCCGTCAGTGGGCAGAGCGCACATCGCCCACAGTCCCCGAGAAGTTGGGGGGAGGGGTCGGCAATTGAACC
GGTGCCTAGAGAAGGTGGCGCGGGGTAAACTGGGAAAGTGATGTCGTGTACTGGCTCCGCCTTTTTCCCGAGGGTGGGGG
AGAACCGTATATAAGTGCAGTAGTCGCCGTGAACGTTCTTTTTCGCAACGGGTTTGCCGCCAGAACACAG
SEQ ID NO: 3-Ef1a short promoter
CTGTGCCTTCTAGTTGCCAGCCATCTGTTGTTTGCCCCTCCCCCGTGCCTTCCTTGACCCTGGAAGGTGCCACTCCCACT
GTCCTTTCCTAATAAAATGAGGAAATTGCATCGCATTGTCTGAGTAGGTGTCATTCTATTCTGGGGGGTGGGGTGGGGCA
GGACAGCAAGGGGGAGGATTGGGAAGACAATAGCAGGCATGCTGGGGATGCGGTGGGCTCTATGG
SEQ ID NO: 4-Bovine Growth Hormone PolyA signal
TCTCTCAGCTGGTACACGGC
SEQ ID NO: 5-TRAC EXON 1 Chr14 22547526 to 22547545 (−) (Human GRCh38-Ensembl release 104-
May 2021)
AGAGTCTCTCAGCTGGTACA
SEQ ID NO: 6-TRAC EXON 1 Chr14 22547530 to 22547549 (−) (Human GRCh38-Ensembl release 104-
May 2021)
GTCCCCTCCACCCCACAGTG
SEQ ID NO: 7-PPP1R12C INTRON1 Chr19: 55, 115,770-55, 115,790 (Human GRCh38-Ensembl release
104 - May 2021)
TCACGTCATCCAGCAGAGAA
SEQ ID NO: 8-B2M EXON 2 CHr15 44715446 to 44715465 (Human GRCh38-Ensembl release 104-May
2021)
GCAAGTCCAGCTTTAAATCA
SEQ ID NO: 9-PTPRC Chr 1 198756152 to 198756171 (+) (Human GRCh38-Ensembl release 104-May
2021)
AACTTGTTTATTGCAGCTTATAATGGTTACAAATAAAGCAATAGCATCACAAATTTCACAAATAAAGCATTTTTTTCACT
GCATTCTAGTTGTGGTTTGTCCAAACTCATCAATGTATCTTA
SEQ ID NO: 10-SV40 PolyA sequence
CGGGCCAAGAGAAGCGGATCCGGC
SEQ ID NO: 11-nucleotide sequence encoding Furin cleavage site and SG linker
RAKRSGSG
SEQ ID NO: 12-peptide sequence encoding Furin cleavage site and SG linker
CAGAACCCTGACCCTGCCGTGTACCAGCTGAGAGACTCTAAATCCAGT
SEQ ID NO: 13-truncated TRAC domain-nucleotide sequence
ATGTCTCTGGGCCTGCTGTGCTGTGGCGTGTTCTCCCTGCTGTGGGCCGGACCTGTGAATGCCGGCGTGACCCAGACCCC
CAAGTTCCGGGTGCTGAAAACCGGCCAGAGCATGACACTGCTGTGCGCCCAGGACATGAACCACGACTACATGTATTGGT
ACAGACAGGACCCCGGCATGGGCCTGCGGCTGATCCACTATTCTGTGGGCGAGGGCACCACCGCCAAGGGCGAAGTGCCT
GATGGCTACAACGTGTCCCGGCTGAAGAAGCAGAACTTCCTGCTGGGCCTGGAAAGCGCCGCTCCTAGCCAGACCAGCGT
GTACTTCTGCGCCAGCAGCTTCACCGACACCCAGTACTTCGGCCCTGGCACCAGACTGACCGTGCTGGAGGACCTGAAGA
ACGTGTTCCCCCCAGAGGTGGCCGTGTTCGAGCCCTCTGAGGCCGAGATCAGCCACACCCAGAAAGCCACCCTGGTCTGC
CTGGCCACCGGCTTCTACCCCGACCACGTGGAACTGTCTTGGTGGGTGAACGGCAAAGAGGTGCACAGCGGCGTCAGCAC
CGACCCTCAGCCCCTGAAAGAGCAGCCCGCCCTGAACGACAGCCGGTACTGCCTGAGCAGCAGACTGCGGGTGTCCGCCA
CCTTCTGGCAGAACCCCCGGAACCACTTCAGATGCCAGGTGCAGTTCTACGGCCTGAGCGAGAACGACGAGTGGACCCAG
GACCGGGCCAAGCCTGTGACCCAGATCGTGTCTGCCGAAGCATGGGGGCGCGCCGATTGCGGCTTCACAAGCGAGAGCTA
CCAGCAGGGCGTGCTGAGCGCCACCATCCTGTACGAGATCCTGCTGGGCAAGGCCACCCTGTACGCCGTGCTGGTGTCCG
CTCTGGTGCTGATGGCCATGGTGAAACGGAAGGACAGCCGGGGC
SEQ ID NO: 14-MAGE-A10 c796 TCR Beta chain nucleotide sequence
MSLGLLCCGVFSLLWAGPVNAGVTQTPKFRVLKTGQSMTLLCAQDMNHDYMYWYRQDPGMGLRLIHYSVGEGTTAKGEVP
DGYNVSRLKKQNFLLGLESAAPSQTSVYFCASSFTDTQYFGPGTRLTVLEDLKNVFPPEVAVFEPSEAEISHTQKATLVC
LATGFYPDHVELSWWVNGKEVHSGVSTDPQPLKEQPALNDSRYCLSSRLRVSATFWQNPRNHFRCQVQFYGLSENDEWTQ
DRAKPVTQIVSAEAWGRADCGFTSESYQQGVLSATILYEILLGKATLYAVLVSALVLMAMVKRKDSRG
SEQ ID NO: 15-MAGE-A10 c796 TCR Beta chain amino acid sequence
ATGATGAAGTCCCTGCGGGTGCTGCTGGTCATCCTGTGGCTGCAGCTGTCCTGGGTCTGGTCCCAGCAGAAAGAGGTGGA
GCAGAACAGCGGCCCTCTGAGCGTGCCCGAGGGCGCTATCGCCAGCCTGAACTGCACCTACAGCGACAGAGGCAGCCAGA
GCTTCTTCTGGTACAGACAGTACAGCGGCAAGAGCCCCGAGCTGATCATGAGCATCTACAGCAACGGCGACAAAGAGGAC
GGCCGGTTCACCGCCCAGCTGAACAAGGCCAGCCAGTACGTGTCCCTGCTGATCCGGGACAGCCAGCCCAGCGACAGCGC
CACCTACCTGTGCGCCGTGAGAGGCACAGGCAGAAGGGCCCTGACATTTGGCAGCGGCACCAGACTGCAGGTGCAGCCCA
ATATCCAGAACCCTGACCCTGCCGTGTACCAGCTGAGAGACTCTAAATCCAGTGACAAGTCTGTCTGCCTATTCACCGAT
TTTGATTCTCAAACAAATGTGTCACAAAGTAAGGATTCTGATGTGTATATCACAGACAAAACTGTGCTAGACATGAGGTC
TATGGACTTCAAGAGCAACAGTGCTGTGGCCTGGAGCAACAAATCTGACTTTGCATGTGCAAACGCCTTCAACAACAGCA
TTATTCCAGAAGACACCTTCTTCCCCAGCCCAGAAAGTTCCTGTGATGTCAAGCTGGTCGAGAAAAGCTTTGAAACAGAT
ACGAACCTAAACTTTCAAAACCTGTCAGTGATTGGGTTCCGAATCCTCCTCCTGAAAGTGGCCGGGTTTAATCTGCTCAT
GACGCTGCGGCTGTGGTCCAGC
SEQ ID NO: 16-MAGE-A10 c796 TCR alpha chain nucleotide sequence
MMKSLRVLLVILWLQLSWVWSQQKEVEQNSGPLSVPEGAIASLNCTYSDRGSQSFFWYRQYSGKSPELIMSIYSNGDKED
GRETAQLNKASQYVSLLIRDSQPSDSATYLCAVRGTGRRALTFGSGTRLQVQPNIQNPDPAVYQLRDSKSSDKSVCLFTD
FDSQTNVSQSKDSDVYITDKTVLDMRSMDFKSNSAVAWSNKSDFACANAFNNSIIPEDTFFPSPESSCDVKLVEKSFETD
TNLNFQNLSVIGFRILLLKVAGENLLMTLRLWSS
SEQ ID NO: 17-MAGE-A10 c796 TCR alpha chain amino acid sequence
CAAATTCACATTGCAAAGAAATGTGGATACAGGAAGGAAAATAAGTTTTATATTCTTGTAATCGATCTATCGTGTATACC
CTCTATGTGGTAGTAACTGTAGATGGTCATCTGGGAATTAATCCTTATTCACAGTGTAAACTTAATTACTCACTAAAATA
TATAAAGCTTTTAATCATGTATGATATTGAGATTTCATATCTTGGTACTTAAAAATGTATCAAATGCTTGCTATGTGCTC
TTGCTATAAAGAGCTAATTGGTATGAGGGAAAGCCAGGTATTTACTAATCAATGTAGTGAGTAAAATGACAGAAAAATTA
TAAGAAGAACATGAATGAGGGCATTTAATTTAAACTTTAGGAATCAAGAAACGCTTCTCGAAGCAGTGATTCCTGCCCTG
ATTCTTAAATAATGTGTAGGCATTAGACAGGAGGATAAGTACAAAACGTGGCATCATGAGCAAAGGCATGGAAATGGCCC
ATGAGCGGAGTGAACACTGGTTTGGGGTTGCTCCAAGGTAAAGTTCAAAAAGTATCCTGCAGTCAACCCTTTAGCACçAT
AAAGAAACTAAATTATTTAGATGTTTTTATGAGAACATATCAAAAAGTACTTTTCTGTCATCCAATACTTCCACAAATAA
ATCATTAGTTCTTGCTAATCTTCATCTGGCATAAAAATAATGACATCAACTTTCTTCATGTAATTTCCCACTTAATTCCT
TTACTAGGAGCAATATCAATTCCTATATGACGTCATTGCCAGCACCTACCCTGCTCAGAATGGACAAGTAAAGAAAAACA
ACCATCAAGAAGATAAAATTGAATTTGATAATGAAGTGGACAAAGTAAAGCAGGATGCTAATTGTGTTAATCCACTTGGT
GCCCCAGAAAAGCTCCCTGAAGCAAAGGAACAGGCTGAAGGTTCTGAACCCACGAGTGGCACTGAGGGGCCAGAACATTC
TGTCAATGGTCCTGCTAGCCCTGCATTGAACCAAGGTTCA
SEQ ID NO: 18-PTPRC Exon 33 Targeting vector-Left Homology Arm (Chr1:198755130-198756201
Human GRCh38-Ensembl release 104-May 2021)
GAAAAGACATAAATGAGGAAACTCCAAACCTCCTGTTAGCTGTTATTTCTATTTTTGTAGAAGTAGGAAGTGAAAATAGG
TATACAGTGGATTAATTAAATGCAGCGAACCAATATTTGTAGAAGGGTTATATTTTACTACTGTGGAAAAATATTTAAGA
TAGTTTTGCCAGAACAGTTTGTACAGACGTATGCTTATTTTAAAATTTTATCTCTTATTCAGTAAAAAACAACTTCTTTG
TAATCGTTATGTGTGTATATGTATGTGTGTATGGGTGTGTGTTTGTGTGAGAGACAGAGAAAGAGAGAGAATTCTTTCAA
GTGAATCTAAAAGCTTTTGCTTTTCCTTTGTTTTTATGAAGAAAAAATACATTTTATATTAGAAGTGTTAACTTAGCTTG
AAGGATCTGTTTTTAAAAATCATAAACTGTGTGCAGACTCAATAAAATCATGTACATTTCTGAAATGACCTCAAGATGTC
CTCCTTGTTCTACTCATATATATCTATCTTATATAGTTTACTATTTTACTTCTAGAGATAGTACATAAAGGTGGTATGTG
TGTGTATGCTACTACAAAAAAGTTGTTAACTAAATTAACATTGGGAAATCTTATATTCCATATATTAGCATTTAGTCCAA
TGTCTTTTTAAGCTTATTTAATTAAAAAATTTCCAGTGAGCTTATCATGCTGTCTTTACATGGGGTTTTCAATTTTGCAT
GCTCGATTATTCCCTGTACAATATTTAAAATTTATTGCTTGATACTTTTGACAACAAATTAGGTTTTGTACAATTGAACT
TAAATAAATGTCATTAAAATAAATAAATGCAATATGTATTAATATTCATTGTATAAAAATAGAAGAATACAAACATATTT
GTTAAATATTTACATATGAAATTTAATATAGCTATTTTTATGGAATTTTTCATTGATATGAAAAATATGATATTGCATAT
GCATAGTTCCCATGTTAAATCCCATTCATAACTTTCATTA
SEQ ID NO: 19-PTPRC Exon 33 Targeting vector-Right Homology Arm (Chromosome 1: 198, 756,
132-198, 757, 230 Human GRCh38-Ensembl release 104-May 2021)
GCTCCCATAGCTCAGTCTGGTCTATCTGCCTGGCCCTGGCCATTGTCACTTTGCGCTGCCCTCCTCTCGCCCCCGAGTGC
CCTTGCTGTGCCGCCGGAACTCTGCCCTCTAACGCTGCCGTCTCTCTCCTGAGTCCGGACCACTTTGAGCTCTACTGGCT
TCTGCGCCGCCTCTGGCCCACTGTTTCCCCTTCCCAGGCAGGTCCTGCTTTCTCTGACCTGCATTCTCTCCCCTGGGCCT
GTGCCGCTTTCTGTCTGCAGCTTGTGGCCTGGGTCACCTCTACGGCTGGCCCAGATCCTTCCCTGCCGCCTCCTTCAGGT
TCCGTCTTCCTCCACTCCCTCTTCCCCTTGCTCTCTGCTGTGTTGCTGCCCAAGGATGCTCTTTCCGGAGCACTTCCTTC
TCGGCGCTGCACCACGTGATGTCCTCTGAGCGGATCCTCCCCGTGTCTGGGTCCTCTCCGGGCATCTCTCCTCCCTCACC
CAACCCCATGCCGTCTTCACTCGCTGGGTTCCCTTTTCCTTCTCCTTCTGGGGCCTGTGCCATCTCTCGTTTCTTAGGAT
GGCCTTCTCCGACGGATGTCTCCCTTGCGTCCCGCCTCCCCTTCTTGTAGGCCTGCATCATCACCGTTTTTCTGGACAAC
CCCAAAGTACCCCGTCTCCCTGGCTTTAGCCACCTCTCCATCCTCTTGCTTTCTTTGCCTGGACACCCCGTTCTCCTGTG
GATTCGGGTCACCTCTCACTCCTTTCATTTGGGCAGCTCCCCTACCCCCCTTACCTCTCTAGTCTGTGCTAGCTCTTCCA
GCCCCCTGTCATGGCATCTTCCAGGGGTCCGAGAGCTCAGCTAGTCTTCTTCCTCCAACCCGGGCCCCTATGTCCACTTC
AGGACAGCATGTTTGCTGCCTCCAGGGATCCTGTGTCCCCGAGCTGGGACCACCTTATATTCCCAGGGCCGGTTAATGTG
GCTCTGGTTCTGGGTACTTTTATCTGTCCCCTCCACCCCA
SEQ ID NO: 20-AAVS1 Intron1 Targeting vector-Left Homology Arm (CHr 19:55115776-
55116775 Human GRCh38-Ensembl release 104-May 2021)
CAGTGGGGCCACTAGGGACAGGATTGGTGACAGAAAAGCCCCATCCTTAGGCCTCCTCCTTCCTAGTCTCCTGATATTGG
GTCTAACCCCCACCTCCTGTTAGGCAGATTCCTTATCTGGTGACACACCCCCATTTCCTGGAGCCATCTCTCTCCTTGCC
AGAACCTCTAAGGTTTGCTTACGATGGAGCCAGAGAGGATCCTGGGAGGGAGAGCTTGGCAGGGGGTGGGAGGGAAGGGG
GGGATGCGTGACCTGCCCGGTTCTCAGTGGCCACCCTGCGCTACCCTCTCCCAGAACCTGAGCTGCTCTGACGCGGCCGT
CTGGTGCGTTTCACTGATCCTGGTGCTGCAGCTTCCTTACACTTCCCAAGAGGAGAAGCAGTTTGGAAAAACAAAATCAG
AATAAGTTGGTCCTGAGTTCTAACTTTGGCTCTTCACCTTTCTAGTCCCCAATTTATATTGTTCCTCCGTGCGTCAGTTT
TACCTGTGAGATAAGGCCAGTAGCCAGCCCCGTCCTGGCAGGGCTGTGGTGAGGAGGGGGGTGTCCGTGTGGAAAACTCC
CTTTGTGAGAATGGTGCGTCCTAGGTGTTCACCAGGTCGTGGCCGCCTCTACTCCCTTTCTCTTTCTCCATCCTTCTTTC
CTTAAAGAGTCCCCAGTGCTATCTGGGACATATTCCTCCGCCCAGAGCAGGGTCCCGCTTCCCTAAGGCCCTGCTCTGGG
CTTCTGGGTTTGAGTCCTTGGCAAGCCCAGGAGAGGCGCTCAGGCTTCCCTGTCCCCCTTCCTCGTCCACCATCTCATGC
CCCTGGCTCTCCTGCCCCTTCCCTACAGGGGTTCCTGGCTCTGCTCTTCAGACTGAGCCCCGTTCCCCTGCATCCCCGTT
CCCCTGCATCCCCCTTCCCCTGCATCCCCCAGAGGCCCCAGGCCACCTACTTGGCCTGGACCCCACGAGAGGCCACCCCA
GCCCTGTCTACCAGGCTGCCTTTTGGGTGGATTCTCCTCCA
SEQ ID NO: 21-AAVS1 Intron1 Targeting vector-Right Homology Arm (Chr 19:55114775-
55115775 Human GRCh38-Ensembl release 104-May 2021)
TTAATTTTCTTTCCTTCACTCCTGTATCGATTTGTGTTGTGTAACAAACCACCCCCAAATTTGGGAGCCTAAACAAATAA
CATTTATTATGGTTCAGTAGTCTAATGATATGCTGGAATGTTCTGCTGGTGCCCTCAGGCTCAGGCAATAGAGACCAGGC
TGACTCATGTGTCTGCCTTCAGCTGATGTGTGCCCTACAGTTTGGCTGGTCTAAAGTGACCTACACTGCCAGTAGGCTGG
CATGTGGCTGCCATTTAGCTATGGCAACAACAGTGAGTGGGCCACATGTCCCTCCTCATCCAGAAGACTAGCCCAGGCCT
ATTCACATTAAAGCAGCAAGTTCCACAAGGGAAAGAAGACTTGTGTGAGACCACTTGAGGCCCAGGCTTAAAAGTGACAC
ACATGTCTTCTTCTGTATGTTATTAGCCAAATAAATAAGTCATAAAGCCTGCCCAGATTCAAGGGGTAGGGAAATAGACT
CCACTTCTTGAGAGGGCCTGCAAATTCACATTGCAAAGAAATGTGGATACAGGAAGGAAAATAAGTTTTATATTCTTGTA
ATCGATCTATCGTGTATACCCTCTATGTGGTAGTAACTGTAGATGGTCATCTGGGAATTAATCCTTATTCACAGTGTAAA
CTTAATTACTCACTAAAATATATAAAGCTTTTAATCATGTATGATATTGAGATTTCATATCTTGGTACTTAAAAATGTAT
CAAATGCTTGCTATGTGCTCTTGCTATAAAGAGCTAATTGGTATGAGGGAAAGCCAGGTATTTACTAATCAATGTAGTGA
GTAAAATGACAGAAAAATTATAAGAAGAACATGAATGAGGGCATTTAATTTAAACTTTAGGAATCAAGAAACGCTTCTCG
AAGCAGTGATTCCTGCCCTGATTCTTAAATAATGTGTAGGCATTAGACAGGAGGATAAGTACAAAACGTGGCATCATGAG
CAAAGGCATGGAAATGGCCCATGAGCGGAGTGAACACTGGTTTGGGGTTGCTCCAAGGTAAAGTTCAAAAAGTATCCTGC
AGTCAACCCTTTAGCACCATAAAGAAACTAAATTATTTAGATGTTTTTATGAGAACATATCAAAAAGTACTTTTCTGTCA
TCCAATACTTCCACAAATAAATCATTAGTTCTTGCTAATCTTCATCTGGCATAAAAATAATGACATCAACTTTCTTCATG
TAATTTCCCACTTAATTCCTTTACTAGGAGCAATATCAATTCCTATATGACGTCATTGCCAGCACCTACCCTGCTCAGAA
TGGACAAGTAAAGAAAAACAACCATCAAGAAGATAAAATTGAATTTGATAATGAAGTGGACAAAGTAAAGCAGGATGCTA
ATTGTGTTAATCCACTTGGTGCCCCAGAAAAGCTCCCTGAAGCAAAGGAACAGGCTGAAGGTTCTGAACCCACGAGTGGC
ACTGAGGGGCCAGAACATTCTGTCAATGGTCCTGCTAGCCCTGCATTGAACCAAGGTTCA
SEQ ID NO: 22 Knock-in of MAGE-B2/A4 ADB959 into PTPRC Exon 33TCR landing pad-
left homology arm Chr1: 198,754,605-198,756,226 (Human GRCh38-Ensembl release 104-
May 2021)
CAGCTGAGAGACTCTAAATCCAGTGACAAGTCTGTCTGCCTATTCACCGATTTTGATTCTCAAACAAATGTGTCACAAAG
TAAGGATTCTGATGTGTATATCACAGACAAAACTGTGCTAGACATGAGGTCTATGGACTTCAAGAGCAACAGTGCTGTGG
CCTGGAGCAACAAATCTGACTTTGCATGTGCAAACGCCTTCAACAACAGCATTATTCCAGAAGACACCTTCTTCCCCAGC
CCAGAAAGTTCCTGTGATGTCAAGCTGGTCGAGAAAAGCTTTGAAACAGATACGAACCTAAACTTTCAAAACCTGTCAGT
GATTGGGTTCCGAATCCTCCTCCTGAAAGTGGCCGGGTTTAATCTGCTCATGACGCTGCGGCTGTGGTCCAGCTGACTGT
GCCTTCTAGTTGCCAGCCATCTGTIGTTTGCCCCTCCCCCGTGCCTTCCTTGACCCTGGAAGGTGCCACTCCCACTGTCC
TTTCCTAATAAAATGAGGAAATTGCATCGCATTGTCTGAGTAGGTGTCATTCTATTCTGGGGGGTGGGGTGGGGCAGGAC
AGCAAGGGGGAGGATTGGGAAGACAATAGCAGGCATGCTGGGGATGCGGTGGGCTCTATGGGAAAAGACATAAATGAGGA
AACTCCAAACCTCCTGTTAGCTGTTATTTCTATTTTTGTAGAAGTAGGAAGTGAAAATAGGTATACAGTGGATTAATTAA
ATGCAGCGAACCAATATTTGTAGAAGGGTTATATTTTACTACTGTGGAAAAATATTTAAGATAGTTTTGCCAGAACAGTT
TGTACAGACGTATGCTTATTTTAAAATTTTATCTCTTATTCAGTAAAAAACAACTTCTTTGTAATCGTTATGTGTGTATA
TGTATGTGTGTATGGGTGTGTGTTTGTGTGAGAGACAGAGAAAGAGAGAGAATTCTTTCAAGTGAATCTAAAAGCTTTTG
CTTTTCCTTTGTTTTTATGAAGAAAAAATACATTTTATATTAGAAGTGTTAACTTAGCTTGAAGGATCTGTTTTTAAAAA
TCATAAACTGTGTGCAGACTCAATAAAATCATGTACATTTCTGAAATGACCTCAAGATGTCCTCCTTGTTCTACTCATAT
ATATCTATCTTATATAGTTTACTATTTTACTTCTAGAGATAGTACATAAAGGTGGTATGTGTGTGTATGCTACTACAAAA
AAGTTGTTAACTAAATTAACATTGGGAAATCTTATATTCCATATATTAGCATTTAGTCCAATGTCTTTTTAAGCTTATTT
AATTAAAAAATTTCCAGTGAGCTTATCATGCTGTCTTTACATGGGGTTTTCAATTTTGCATGCTCGATTATTCCCTGTAC
AATATTTAAAATTTATTGCTTGATACTTTTGACAACAAATTAGGTTTTGTACAATTGAACTTAAATAAATGTCATTAAAA
TAAATAAATGCAATATGTATTAATATTCATTGTATAAAAATAGAAGAATACAAACATATTTGTTAAATATTTACATATGA
AATTTAATATAGCTATTTTTATGGAATTTTTCATTGATATGAAAAATATGATATTGCATATGCATAGTTCCCATGTTAAA
TCCCATTCATAACTTTCATTA
SEQ ID NO: 23 Knock-in of MAGE-B2/A4 ADB959 into PTPRC Exon 33TCR landing pad-
right homology arm; include TRAC domain sequence (nucleotides 1-396), BGH polyA signal
(397- 621) and nucleotides corresponding to Chromosome 1: 198, 756, 132-198, 757, 230 Human
GRCh38-Ensembl release 104-May 2021)
AGTTCAGGTTCAAGAGCTAAAAGGAGCGGATCAGGT
SEQ ID NO: 24-FURIN SG LINKER
GGCAGCCGGGCCAAGAGATCTGGATCCGGC
SEQ ID NO: 25-FURIN SG LINKER
SSGSRAKRSGS
SEQ ID NO: 26 FURIN SG LINKER
GSRAKRSGSG
SEQ ID NO: 27 FURIN SG LINKER
GAGGGCAGAGGCAGCCTGCTGACATGTGGCGACGTGGAAGAAAACCCTGGCCCT
SEQ ID NO: 28-T2A skip like sequence nucleotide sequence
EGRGSLLTCGDVEENPGP
SEQ ID NO: 29-T2A skip like sequence amino acid sequence
GCTACCAACTTTAGCCTGCTGAAGCAGGCCGGGGACGTGGAAGAAAACCCTGGCCCT
SEQ ID NO: 30-P2A skip like sequence nucleotide sequence
ATNFSLLKQAGDVEENPGP
SEQ ID NO: 31-P2A skip like sequence amino acid sequence
ATGGCCAGCCTGCTGTTCTTCTGCGGCGCCTTCTACCTGCTGGGCACCGGCTCTATGGATGCCGACGTGACCCAGACCCC
CCGGAACAGAATCACCAAGACCGGCAAGCGGATCATGCTGGAATGCTCCCAGACCAAGGGCCACGACCGGATGTACTGGT
ACAGACAGGACCCTGGCCTGGGCCTGCGGCTGATCTACTACAGCTTCGACGTGAAGGACATCAACAAGGGCGAGATCAGC
GACGGCTACAGCGTGTCCAGACAGGCTCAGGCCAAGTTCAGCCTGTCCCTGGAAAGCGCCATCCCCAACCAGACCGCCCT
GTACTTTTGTGCCACAAGCGGCCAGGGCGCCTACAACGAGCAGTTCTTTGGCCCTGGCACCCGGCTGACAGTGCTGGAAG
ATCTGAAGAACGTGTTCCCCCCAGAGGTGGCCGTGTTCGAGCCTTCTGAGGCCGAAATCAGCCACACCCAGAAAGCCACA
CTCGTGTGTCTGGCCACCGGCTTCTACCCCGACCACGTGGAACTGTCTTGGTGGGTCAACGGCAAAGAGGTGCACAGCGG
CGTGTCCACCGATCCCCAGCCTCTGAAAGAACAGCCCGCCCTGAACGACAGCCGGTACTGCCTGAGCAGCAGACTGAGAG
TGTCCGCCACCTTCTGGCAGAACCCCAGAAACCACTTCAGATGCCAGGTGCAGTTTTACGGCCTGAGCGAGAACGACGAG
TGGACCCAGGACAGAGCCAAGCCCGTGACACAGATCGTGTCTGCCGAAGCTTGGGGGCGCGCCGATTGTGGCTTTACCAG
CGAGAGCTACCAGCAGGGCGTGCTGAGCGCCACCATCCTGTACGAGATCCTGCTGGGAAAGGCCACACTGTACGCCGTGC
TGGTGTCTGCCCTGGTGCTGATGGCCATGGTCAAGCGGAAGGACAGCCGGGGC
SEQ ID NO: 32-ADB959 TCR beta chain nucleotide sequence
ATGAAGAAGCACCTGACCACCTTTCTCGTGATCCTGTGGCTGTACTTCTACCGGGGCAACGGCAAGAACCAGGTGGAACA
GAGCCCCCAGAGCCTGATCATCCTGGAAGGCAAGAACTGCACCCTGCAGTGCAACTACACCGTGTCCCCCTTCAGCAACC
TGCGGTGGTACAAGCAGGACACCGGCAGAGGCCCTGTGTCCCTGACCATCGTGACCTTCAGCGAGAACACCAAGAGCAAC
GGCCGGTACACCGCCACCCTGGACGCCGATACAAAGCAGAGCAGCCTGCACATCACCGCCAGCCAGCTGAGCGATAGCGC
CAGCTACATCTGCGTGGTGTCCGGCGGCACAGACAGCTGGGGCAAGCTGCAGTTTGGCGCCGGAACACAGGTGGTCGTGA
CCCCCGACATCCAGAACCCTGACCCTGCAGTATATCAGCTGAGAGACTCTAAATCCAGTGACAAGTCTGTCTGCCTATTC
ACCGATTTTGATTCTCAAACAAATGTGTCACAAAGTAAGGATTCTGATGTGTATATCACAGACAAAACTGTGCTAGACAT
GAGGTCTATGGACTTCAAGAGCAACAGTGCTGTGGCCTGGAGCAACAAATCTGACTTTGCATGTGCAAACGCCTTCAACA
ACAGCATTATTCCAGAAGACACCTTCTTCCCCAGCCCAGAAAGTTCCTGTGATGTCAAGCTGGTCGAGAAAAGCTTTGAA
ACAGATACGAACCTAAACTTTCAAAACCTGTCAGTGATTGGGTTCCGAATCCTCCTCCTGAAAGTGGCCGGGTTTAATCT
GCTCATGACGCTGCGGCTGTGGTCCAGCTGA
SEQ ID NO: 33-ADB959 TCR alpha chain nucleotide sequence
EQYQFLYDVIASTYPAQNGQVKKNNHQEDKIEFDNEVDKVKQDANCVNPLGAPEKLPEAKEQAEGSEPTSGTEGPEHSVN
GPASPALNQGSSSGSRAKRSGSGEGRGSLLTCGDVEENPGPMASLLFFCGAFYLLGTGSMDADVTQTPRNRITKTGKRIM
LECSQTKGHDRMYWYRQDPGLGLRLIYYSFDVKDINKGEISDGYSVSRQAQAKFSLSLESAIPNQTALYFCATSGQGAYN
EQFFGPGTRLTVLEDLKNVFPPEVAVFEPSEAEISHTOKATLVCLATGFYPDHVELSWWVNGKEVHSGVSTDPQPLKEQP
ALNDSRYCLSSRLRVSATFWQNPRNHFRCQVQFYGLSENDEWTQDRAKPVTQIVSAEAWGRADCGFTSESYQQGVLSATI
LYEILLGKATLYAVLVSALVLMAMVKRKDSRGGSRAKRSGSGATNFSLLKQAGDVEENPGPMKKHLTTFLVILWLYFYRG
NGKNQVEQSPQSLIILEGKNCTLQCNYTVSPFSNLRWYKQDTGRGPVSLTIVTFSENTKSNGRYTATLDADTKQSSLHIT
ASQLSDSASYICVVSGGTDSWGKLQFGAGTQVVVTPDIQNPDPAVYQLRDSKSSDKSVCLFTDFDSQTNVSQSKDSDVYI
TDKTVLDMRSMDFKSNSAVAWSNKSDFACANAFNNSIIPEDTFFPSPESSCDVKLVEKSFETDINLNFQNLSVIGFRILL
LKVAGFNLLMTLRLWSS
SEQ ID NO: 34-Translated sequence PTPRC Exon 33_T2A ADB959_TCRB_P2A_TCRa
CCCGCGCCGTGCTGGACTCCACCAACGCCGACGGTATCAGCGCCCTGCACCAGGTCAGCGCCCCCCGCCCGGCGTCTCCC
GGGGCCAGGTCCACCCTCTGCTGCGCCACCTGGGGCATCCTCCTTCCCCGTTGCCAGTCTCGATCCGCCCCGTCGTTCCT
GGCCCTGGGCTTTGCCACCCTATGCTGACACCCCGTCCCAGTCCCCCTTACCATTCCCCTTCGACCACCCCACTTCCGAA
TTGGAGCCGCTTCAACTGGCCCTGGGCTTAGCCACTCTGTGCTGACCACTCTGCCCCAGGCCTCCTTACCATTCCCCTTC
GACCTACTCTCTTCCGCATTGGAGTCGCTTTAACTGGCCCTGGCTTTGGCAGCCTGTGCTGACCCATGCAGTCCTCCTTA
CCATCCCTCCCTCGACTTCCCCTCTTCCGATGTTGAGCCCCTCCAGCCGGTCCTGGACTTTGTCTCCTTCCCTGCCCTGC
CCTCTCCTGAACCTGAGCCAGCTCCCATAGCTCAGTCTGGTCTATCTGCCTGGCCCTGGCCATTGTCACTTTGCGCTGCC
CTCCTCTCGCCCCCGAGTGCCCTTGCTGTGCCGCCGGAACTCTGCCCTCTAACGCTGCCGTCTCTCTCCTGAGTCCGGAC
CACTTTGAGCTCTACTGGCTTCTGCGCCGCCTCTGGCCCACTGTTTCCCCTTCCCAGGCAGGTCCTGCTTTCTCTGACCT
GCATTCTCTCCCCTGGGCCTGTGCCGCTTTCTGTCTGCAGCTTGTGGCCTGGGTCACCTCTACGGCTGGCCCAGATCCTT
CCCTGCCGCCTCCTTCAGGTTCCGTCTTCCTCCACTCCCTCTTCCCCTTGCTCTCTGCTGTGTTGCTGCCCAAGGATGCT
CTTTCCGGAGCACTTCCTTCTCGGCGCTGCACCACGTGATGTCCTCTGAGCGGATCCTCCCCGTGTCTGGGTCCTCTCCG
GGCATCTCTCCTCCCTCACCCAACCCCATGCCGTCTTCACTCGCTGGGTTCCCTTTTCCTTCTCCTTCTGGGGCCTGTGC
CATCTCTCGTTTCTTAGGATGGCCTTCTCCGACGGATGTCTCCCTTGCGTCCCGCCTCCCCTTCTTGTAGGCCTGCATCA
TCACCGTTTTTCTGGACAACCCCAAAGTACCCCGTCTCCCTGGCTTTAGCCACCTCTCCATCCTCTTGCTTTCTTTGCCT
GGACACCCCGTTCTCCTGTGGATTCGGGTCACCTCTCACTCCTTTCATTTGGGCAGCTCCCCTACCCCCCTTACCTCTCT
AGTCTGTGCTAGCTCTTCCAGCCCCCTGTCATGGCATCTTCCAGGGGTCCGAGAGCTCAGCTAGTCTTCTTCCTCCAACC
CGGGCCCCTATGTCCACTTCAGGACAGCATGTTTGCTGCCTCCAGGGATCCTGTGTCCCCGAGCTGGGACCACCTTATAT
TCCCAGGGCCGGTTAATGTGGCTCTGGTTCTGGGTACTTTTATCTGTCCCCTCCACCCCA
SEQ ID NO: 35-Knock-in of MAGE-B2/A4 ADB959 into AAVS1 TCR landing pad-left homology
arm (Chr 19: 55,115,701-55, 117,349 Human GRCh38-Ensembl release 104-May 2021)
GCTCCGGTGCCCGTCAGTGGGCAGAGCGCACATCGCCCACAGTCCCCGAGAAGTTGGGGGGAGGGGTCGGCAATTGAACC
GGTGCCTAGAGAAGGTGGCGCGGGGTAAACTGGGAAAGTGATGTCGTGTACTGGCTCCGCCTTTTTCCCGAGGGTGGGGG
AGAACCGTATATAAGTGCAGTAGTCGCCGTGAACGTTCTTTTTCGCAACGGGTTTGCCGCCAGAACACAG
SEQ ID NO: 36-EF1A short promoter
ATGGCCAGCCTGCTGTTCTTCTGCGGCGCCTTCTACCTGCTGGGCACCGGCTCTATGGATGCCGACGTGACCCAGACCCC
CCGGAACAGAATCACCAAGACCGGCAAGCGGATCATGCTGGAATGCTCCCAGACCAAGGGCCACGACCGGATGTACTGGT
ACAGACAGGACCCTGGCCTGGGCCTGCGGCTGATCTACTACAGCTTCGACGTGAAGGACATCAACAAGGGCGAGATCAGC
GACGGCTACAGCGTGTCCAGACAGGCTCAGGCCAAGTTCAGCCTGTCCCTGGAAAGCGCCATCCCCAACCAGACCGCCCT
GTACTTTTGTGCCACAAGCGGCCAGGGCGCCTACAACGAGCAGTTCTTTGGCCCTGGCACCCGGCTGACAGTGCTGGAAG
ATCTGAAGAACGTGTTCCCCCCAGAGGTGGCCGTGTTCGAGCCTTCTGAGGCCGAAATCAGCCACACCCAGAAAGCCACA
CTCGTGTGTCTGGCCACCGGCTTCTACCCCGACCACGTGGAACTGTCTTGGTGGGTCAACGGCAAAGAGGTGCACAGCGG
CGTGTCCACCGATCCCCAGCCTCTGAAAGAACAGCCCGCCCTGAACGACAGCCGGTACTGCCTGAGCAGCAGACTGAGAG
TGTCCGCCACCTTCTGGCAGAACCCCAGAAACCACTTCAGATGCCAGGTGCAGTTTTACGGCCTGAGCGAGAACGACGAG
TGGACCCAGGACAGAGCCAAGCCCGTGACACAGATCGTGTCTGCCGAAGCTTGGGGGCGCGCCGATTGTGGCTTTACCAG
CGAGAGCTACCAGCAGGGCGTGCTGAGCGCCACCATCCTGTACGAGATCCTGCTGGGAAAGGCCACACTGTACGCCGTGC
TGGTGTCTGCCCTGGTGCTGATGGCCATGGTCAAGCGGAAGGACAGCCGGGGC
SEQ ID NO: 37-ADB959 TCR Beta Chain nucleotide sequence
ATGAAGAAGCACCTGACCACCTTTCTCGTGATCCTGTGGCTGTACTICTACCGGGGCAACGGCAAGAACCAGGTGGAACA
GAGCCCCCAGAGCCTGATCATCCTGGAAGGCAAGAACTGCACCCTGCAGTGCAACTACACCGTGTCCCCCTTCAGCAACC
TGCGGTGGTACAAGCAGGACACCGGCAGAGGCCCTGTGTCCCTGACCATCGTGACCTTCAGCGAGAACACCAAGAGCAAC
GGCCGGTACACCGCCACCCTGGACGCCGATACAAAGCAGAGCAGCCTGCACATCACCGCCAGCCAGCTGAGCGATAGCGC
CAGCTACATCTGCGTGGTGTCCGGCGGCACAGACAGCTGGGGCAAGCTGCAGTTTGGCGCCGGAACACAGGTGGTCGTGA
CCCCCGACATCCAGAACCCTGACCCTGCAGTATATCAGCTGAGAGACTCTAAATCCAGTGACAAGTCTGTCTGCCTATTC
ACCGATTTTGATTCTCAAACAAATGTGTCACAAAGTAAGGATTCTGATGTGTATATCACAGACAAAACTGTGCTAGACAT
GAGGTCTATGGACTTCAAGAGCAACAGTGCTGTGGCCTGGAGCAACAAATCTGACTTTGCATGTGCAAACGCCTTCAACA
ACAGCATTATTCCAGAAGACACCTTCTTCCCCAGCCCAGAAAGTTCCTGTGATGTCAAGCTGGTCGAGAAAAGCTTTGAA
ACAGATACGAACCTAAACTTTCAAAACCTGTCAGTGATTGGGTTCCGAATCCTCCTCCTGAAAGTGGCCGGGTTTAATCT
GCTCATGACGCTGCGGCTGTGGTCCAGCTGA
SEQ ID NO: 38-ADB959 TCR Alpha Chain nucleotide sequence
MASLLFFCGAFYLLGTGSMDADVTQTPRNRITKTGKRIMLECSQTKGHDRMYWYRQDPGLGLRLIYYSFDVKDINKGEIS
DGYSVSRQAQAKFSLSLESAIPNQTALYFCATSGQGAYNEQFFGPGTRLTVLEDLKNVFPPEVAVFEPSEAEISHTQKAT
LVCLATGFYPDHVELSWWVNGKEVHSGVSTDPQPLKEQPALNDSRYCLSSRLRVSATFWQNPRNHFRCQVQFYGLSENDE
WTQDRAKPVTQIVSAEAWGRADCGFTSESYQQGVLSATILYEILLGKATLYAVLVSALVLMAMVKRKDSRGGSRAKRSGS
GATNFSLLKQAGDVEENPGPMKKHLTTFLVILWLYFYRGNGKNQVEQSPQSLIILEGKNCTLQCNYTVSPFSNLRWYKQD
TGRGPVSLTIVTFSENTKSNGRYTATLDADTKQSSLHITASQLSDSASYICVVSGGTDSWGKLQFGAGTQVVVTPDIQNP
DPAVYQLRDSKSSDKSVCLFTDFDSQTNVSQSKDSDVYITDKTVLDMRSMDFKSNSAVAWSNKSDFACANAFNNSIIPED
TFFPSPESSCDVKLVEKSFETDTNLNFQNLSVIGFRILLLKVAGENLLMTLRLWSS-
SEQ ID NO: 39-ADB959 TCRB_P2A_TCRA AMINO ACID SEQUENCE
CTGTGCCTTCTAGTTGCCAGCCATCTGTTGTTTGCCCCTCCCCCGTGCCTTCCTTGACCCTGGAAGGTGCCACTCCCACT
GTCCTTTCCTAATAAAATGAGGAAATTGCATCGCATTGTCTGAGTAGGTGTCATTCTATTCTGGGGGGTGGGGTGGGGCA
GGACAGCAAGGGGGAGGATTGGGAAGACAATAGCAGGCATGCTGGGGATGCGGTGGGCTCTATGG
SEQ ID NO: 40-BGH POLY A SIGNAL
CAGCTGAGAGACTCTAAATCCAGTGACAAGTCTGTCTGCCTATTCACCGATTTTGATTCTCAAACAAATGTGTCACAAAG
TAAGGATTCTGATGTGTATATCACAGACAAAACTGTGCTAGACATGAGGTCTATGGACTTCAAGAGCAACAGTGCTGTGG
CCTGGAGCAACAAATCTGACTTTGCATGTGCAAACGCCTTCAACAACAGCATTATTCCAGAAGACACCTTCTTCCCCAGC
CCAGAAAGTTCCTGTGATGTCAAGCTGGTCGAGAAAAGCTTTGAAACAGATACGAACCTAAACTTTCAAAACCTGTCAGT
GATTGGGTTCCGAATCCTCCTCCTGAAAGTGGCCGGGTTTAATCTGCTCATGACGCTGCGGCTGTGGTCCAGCTGACTGT
GCCTTCTAGTTGCCAGCCATCTGTTGTTTGCCCCTCCCCCGTGCCTTCCTTGACCCTGGAAGGTGCCACTCCCACTGTCC
TTTCCTAATAAAATGAGGAAATTGCATCGCATTGTCTGAGTAGGTGTCATTCTATTCTGGGGGGTGGGGTGGGGCAGGAC
AGCAAGGGGGAGGATTGGGAAGACAATAGCAGGCATGCTGGGGATGCGGTGGGCTCTATGGCAGTGGGGCCACTAGGGAC
AGGATTGGTGACAGAAAAGCCCCATCCTTAGGCCTCCTCCTTCCTAGTCTCCTGATATTGGGTCTAACCCCCACCTCCTG
TTAGGCAGATTCCTTATCTGGTGACACACCCCCATTTCCTGGAGCCATCTCTCTCCTTGCCAGAACCTCTAAGGTTTGCT
TACGATGGAGCCAGAGAGGATCCTGGGAGGGAGAGCTTGGCAGGGGGTGGGAGGGAAGGGGGGGATGCGTGACCTGCCCG
GTTCTCAGTGGCCACCCTGCGCTACCCTCTCCCAGAACCTGAGCTGCTCTGACGCGGCCGTCTGGTGCGTTTCACTGATC
CTGGTGCTGCAGCTTCCTTACACTTCCCAAGAGGAGAAGCAGTTTGGAAAAACAAAATCAGAATAAGTTGGTCCTGAGTT
CTAACTTTGGCTCTTCACCTTTCTAGTCCCCAATTTATATTGTTCCTCCGTGCGTCAGTTTTACCTGTGAGATAAGGCCA
GTAGCCAGCCCCGTCCTGGCAGGGCTGTGGTGAGGAGGGGGGTGTCCGTGTGGAAAACTCCCTTTGTGAGAATGGTGCGT
CCTAGGTGTTCACCAGGTCGTGGCCGCCTCTACTCCCTTTCTCTTTCTCCATCCTTCTTTCCTTAAAGAGTCCCCAGTGC
TATCTGGGACATATTCCTCCGCCCAGAGCAGGGTCCCGCTTCCCTAAGGCCCTGCTCTGGGCTTCTGGGTTTGAGTCCTT
GGCAAGCCCAGGAGAGGCGCTCAGGCTTCCCTGTCCCCCTTCCTCGTCCACCATCTCATGCCCCTGGCTCTCCTGCCCCT
TCCCTACAGGGGTTCCTGGCTCTGCTCTTCAGACTGAGCCCCGTTCCCCTGCATCCCCGTTCCCCTGCATCCCCCTTCCC
CTGCATCCCCCAGAGGCCCCAGGCCACCTACTTGGCCTGGACCCCACGAGAGGCCACCCCAGCCCTGTCTACCAGGCTGC
CTTTTGGGTGGATTCTCCTCCA
SEQ ID NO: 41-Knock-in of MAGE-B2/A4 ADB959 into AAVS1 TCR landing pad-right
homology arm-contains TRAC domain sequence (nucleotides 1-396), BGH polyA signal
(397-621) and nucleotides; corresponding to Chr19: 55, 114, 725-55, 115, 825 Human
GRCh38-Ensembl release 104-May 2021)
5′-TTCAGGTTTACTCACGTCATCCAGCAGAGAATGGAAAGTCAGGCTCCGGTGCCCGTCA-3′
SEQ ID NO: 42 (B2M FWD target sequence underlined)
5′-TGACTTTCCATTCTCTGCTGGATGACGTGAGTAAACCTGAAAACTTGTTTATTGCAGCTTATAATGG-3′
SEQ ID NO: 43 (B2M FWD target sequence underlined)
5′ CGACCTTTGGTCGCCCGGCCTCAGTGAGCGAGCGCGCAGAGAGGGAGTGGCCAACTCCATCACTAGGGGTTCCTGCGG
CCGCTGGGTTCCCTTTTCCTTC 3′
SEQ ID NO: 44 (FWD; binding Chr19: 55116254-55116274 GRCh38. p14)
5′-GACTTTCCATTCTCTGCTGGATGACGTGAGTAAACCTGAATGTGGGGTGGAGGGGACAG-3′
SEQ ID NO: 45 (REV binding Chr 19: 55115774-55115792 GRCh38. p14).
5′-TCAGGTTTACTCACGTCATCCAGCAGAGAATGGAAAGTCAGTGGGGCCACTAGGGACAGGATTG-3′
SEQ ID NO: 46 (FWD binding Chr19: 55115750-55115773 GRCh38. p14)
5′-GAGGGAGTGGCCAACTCCATCACTAGGGGTTCCTGCGGCCGCTACTGGCCTTATCTCACAG-3′
SEQ ID NO: 47 (REV binding Chr19: 55115274-55115292 GRCh38. p14)
5′ GTCGATCCTACCATCCACTCGACACACCCGCCAGCGGCCGCTGCCAAGCTTGCCGCCACCATGAAGAAGCACCTGACC
ACCTTTCTCGTGATC-3′
SEQ ID NO: 48 (FWD)
5′-CCAATCCTGTCCCTAGTGGCCCCACTGACTTTCCATTCCCATAGAGCCCACCGCATCCCCAG-3′
SEQ ID NO: 49 (REV)
5′ CGACCTTTGGTCGCCCGGCCTCAGTGAGCGAGCGCGCAGAGAGGGAGTGGCCAACTCCATCACTAGGGGTTCCTGCGG
CCGCTGGGTTCCCTTTTCCTTC-3′
SEQ ID NO: 50 (FWD)
5′ GTGGGCGATGTGCGCTCTGCCCACTGACGGGCACCGGAGCCTCTGCTGGATGACGTGAGTAAACCTGAATGTGGGGTG
GAGGGGACAG-3′
SEQ ID NO: 51 (REV)
5′-GAATGGAAAGTCAGTGGGGCCACTAGGGACAGGATTGG-3′
SEQ ID NO: 52 (FWD)
5′ TTTGGTCGCCCGGCCTCAGTGAGCGAGCGAGCGCGCAGAGAGGGAGTGGCCAACTCCATCACTAGGGGTTCCTGCGGC
CGCTACTGGCCTTATCTCACAG-3′
SEQ ID NO: 53 (REV)
5′-GGATGCTCTTTCCGGAGCAC-3′
SEQ ID NO: 54 (5′ FWD; binding Chr19: 55116402-55116383 GRCh38. p14)
5′-GCACCGGTTCAATTGCCGAC-3′
SEQ ID NO: 55 (5′ REV; binding EF1-a of ADB00794_001)
5′-TGGTGAACACCTAGGACGCA-3′
SEQ ID NO: 56 (3′ FWD; binding Chr19: 55115182-55115201 GRCh38. p14)
5′-GGCTCTCGGAGAATGACGA-3′
SEQ ID NO: 57 (3′ REV; binding A2M10 of ADB00794_001)
GGCTCCGGTGCCCGTCAGTGGGCAGAGCGCACATCGCCCACAGTCCCCGAGAAGTTGGGGGGAGGGGTCGGCAATTGAAC
CGGTGCCTAGAGAAGGTGGCGCGGGGTAAACTGGGAAAGTGATGTCGTGTACTGGCTCCGCCTTTTTCCCGAGGGTGGGG
GAGAACCGTATATAAGTGCACTAGTCGCCGTGAACGTTCTTTTTCGCAACGGGTTTGCCGCCAGAACACAGGTAAGTGCC
GTGTGTGGTTCCCGCGGGCCTGGCCTCTTTACGGGTTATGGCCCTTGCGTGCCTTGAATTACTTCCACCTGGCTGCAGTA
CGTGATTCTTGATCCCGAGCTTCGGGTTGGAAGTGGGTGGGAGAGTTCGTGGCCTTGCGCTTAAGGAGCCCCTTCGCCTC
GTGCTTGAGTTGTGGCCTGGCCTGGGCGCTGGGGCCGCCGCGTGCGAATCTGGTGGCACCTTCGCGCCTGTCTCGCTGCT
TTCGATAAGTCTCTAGCCATTTAAAATTTTTGATGACTGCTGCGACGCTTTTTTTCTGGCAACGATAGTCTTGTAAATGC
GGGCCAAGATCAGCACACTGGTATTTCGGTTTTTGGGGCCGCGGGCGGCGACGGGGCCCGTGCGTCCCAGCGCACATGTT
CGGCGAGGCGGGGCCTGCGAGCGCGGCCACCCAGAATCGGACGGGGGTAGTCTCAAGCTGCCCGGCCTGCTCTGGTGCCT
GGCCTCGCGCCGCCGTGTATCGCCCCGCCCTGGGCGGCAAGGCTGGCCCGGTCGGCACCAGTTGCGTGAGCGGAAAGATG
GCCGCTTCCCGGCCCTGCTGCAGGGAGCACAAAATGGAGGACGCGGCGCTCGGGAGAGCGGGCGGGTGAGTCACCCACAC
AAAGGAAAAGGGCCTTTCCGTCCTCAGCCGTCGCTTCATGTGACTCCACGGAGTACCGGGCGCCGTCCAGGCACCTCGAT
TAGTTCTCCAGCTTTTGGAGTACGTCGTCTTTAGGTTGGGGGGAGGGGTTTTATGCGATGGAGTTTCCCCACACTGAGTG
GGTGGAGACTGAAGTTAGGCCAGCTTGGCACTTGATGTAATTCTCCTTGGAATTTGCCCTTTTTGAGTTTGGATCTTGGT
TCATTCTCAAGCCTCAGACAGTGGTTCAAAGTTTTTTTCTTCCATTTCAGGTGTCGTGAAAACTACCCCTAAAAGCCAAA

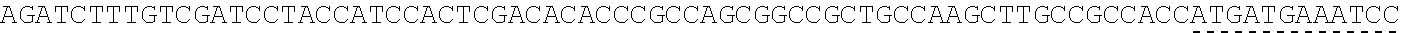

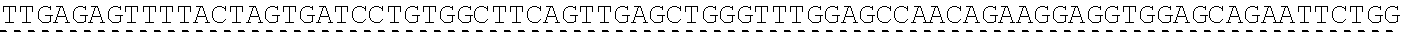

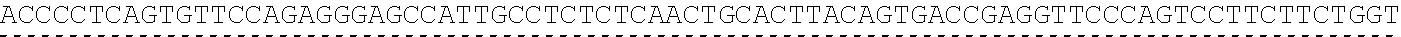

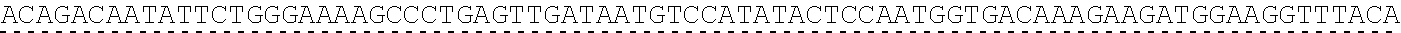

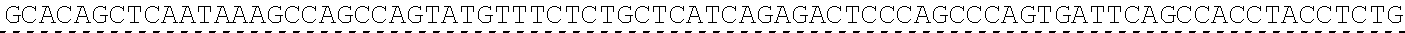

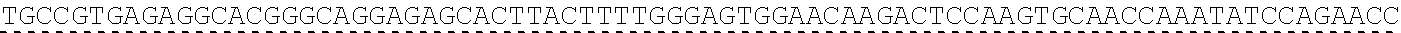

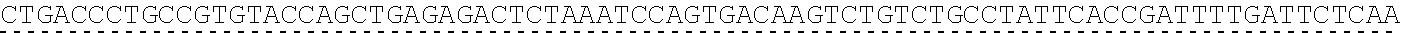

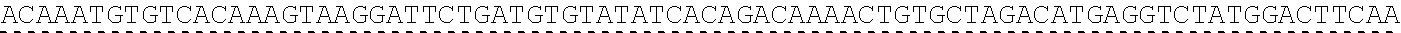

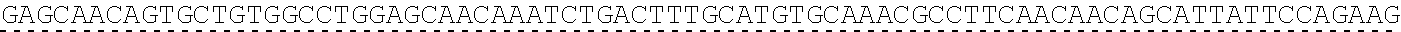

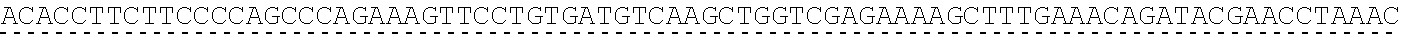

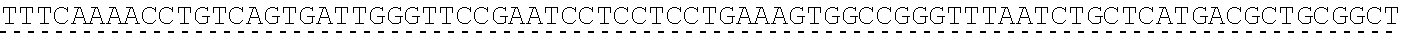

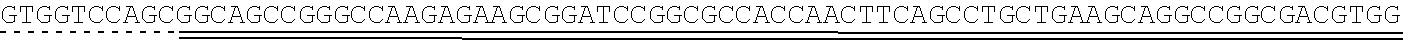

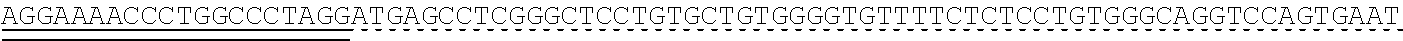

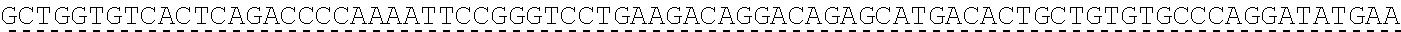

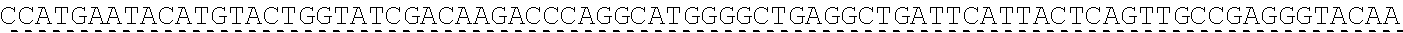

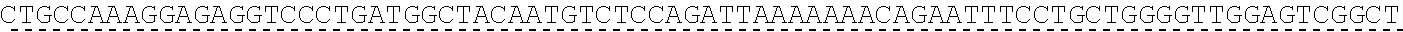

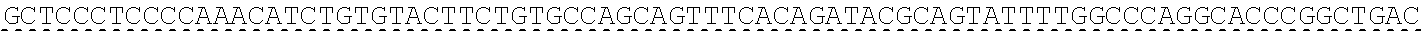

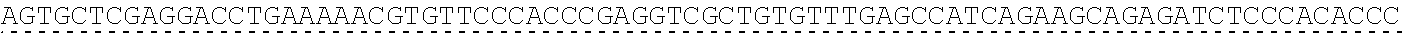

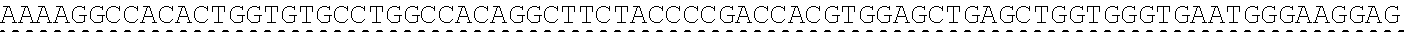

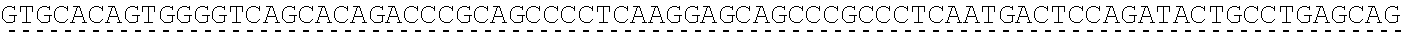

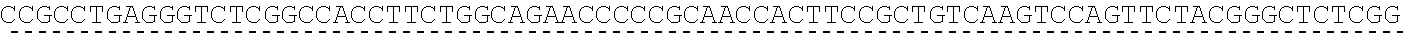

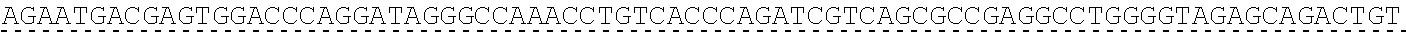

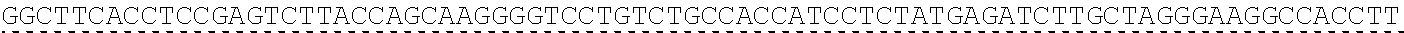

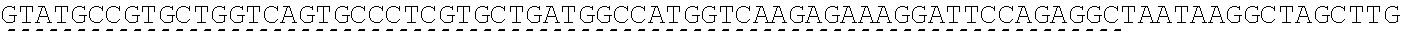

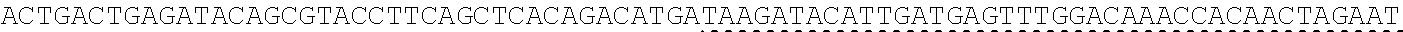

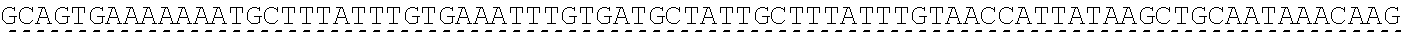

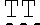

SEQ ID NO: 58 (A2M10 TCR cassette ADB00794_001 Underline = EF-1a promoter; Dotted
underline = the SV40 polyadenylation signal Dashed underline = A2M10 TCR; Double
underline = Furin cleavage site, P2A)
CCTGCAGGCAGCTGCGCGCTCGCTCGCTCACTGAGGCCGCCCGGGCGTCGGGCGACCTTTGGTCGCCCGGCCTCAGTGAG
CGAGCGCGCAGAGAGGGAGTGGCCAACTCCATCACTAGGGGTTCCTGCGGCCACTGGGTTCCCTTTTCCTTCTCCTTCTG
GGGCCTGTGCCATCTCTCGTTTCTTAGGATGGCCTTCTCCGACGGATGTCTCCCTTGCGTCCCGCCTCCCCTTCTTGTAG
GCCTGCATCATCACCGTTTTTCTGGACAACCCCAAAGTACCCCGTCTCCCTGGCTTTAGCCACCTCTCCATCCTCTTGCT
TTCTTTGCCTGGACACCCCGTTCTCCTGTGGATTCGGGTCACCTCTCACTCCTTTCATTTGGGCAGCTCCCCTACCCCCC
TTACCTCTCTAGTCTGTGCTAGCTCTTCCAGCCCCCTGTCATGGCATCTTCCAGGGGTCCGAGAGCTCAGCTAGTCTTCT
TCCTCCAACCCGGGCCCCTATGTCCACTTCAGGACAGCATGTTTGCTGCCTCCAGGGATCCTGTGTCCCCGAGCTGGGAC
CACCTTATATTCCCAGGGCCGGTTAATGTGGCTCTGGTTCTGGGTACTTTTATCTGTCCCCTCCACCCCACATTCAGGTT

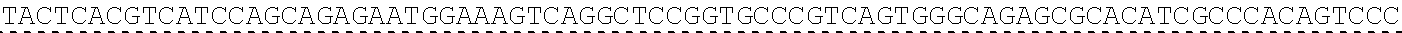

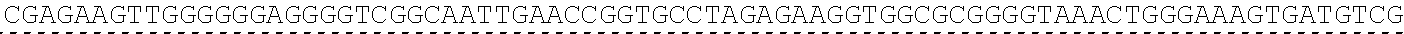

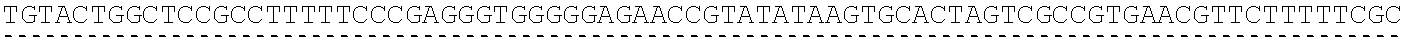

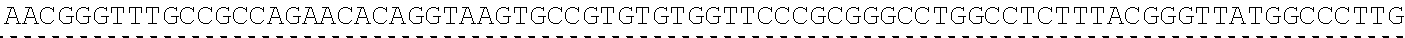

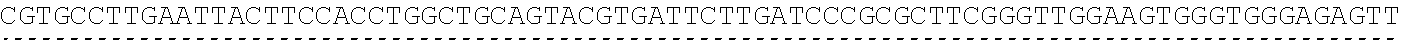

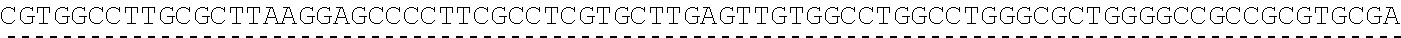

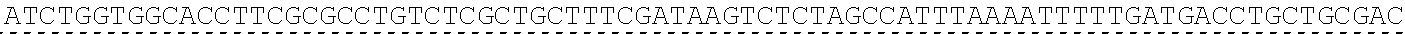

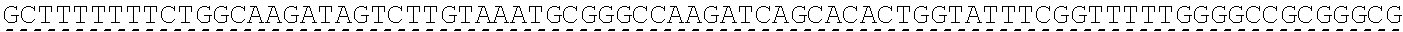

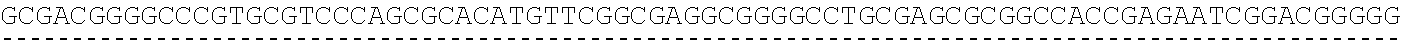

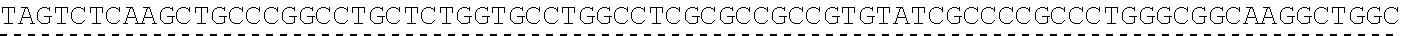

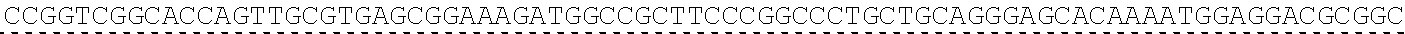

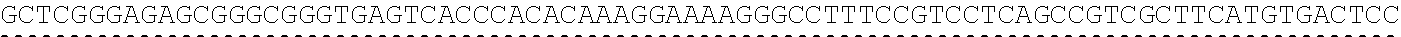

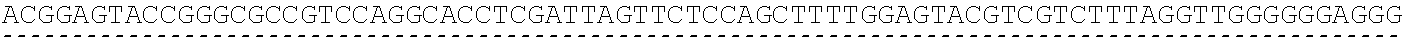

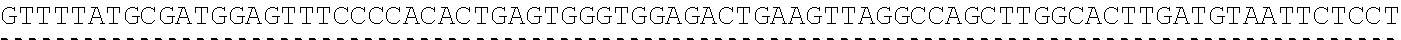

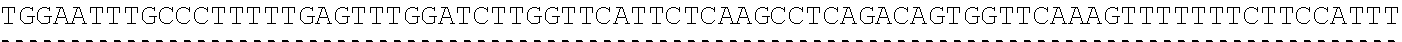

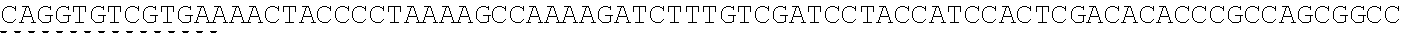

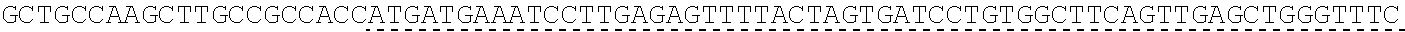

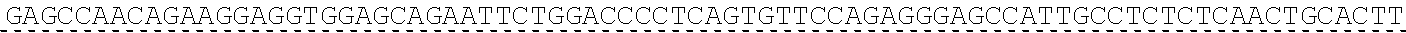

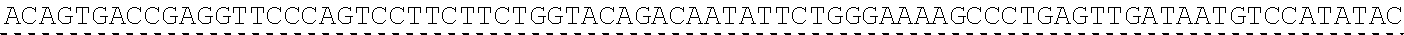

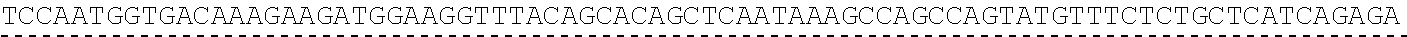

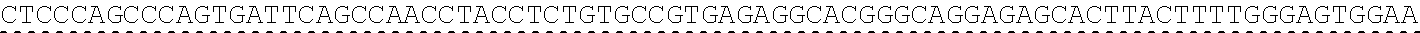

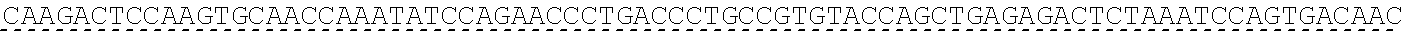

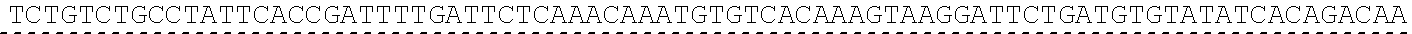

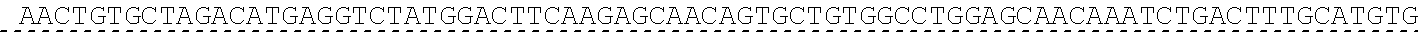

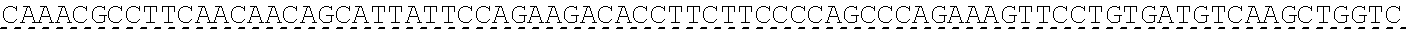

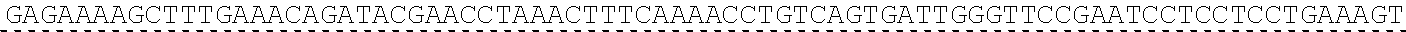

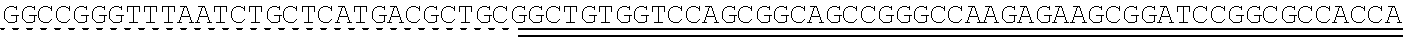

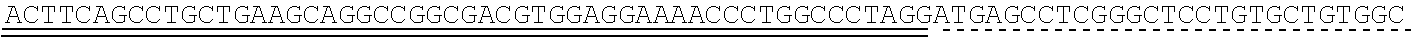

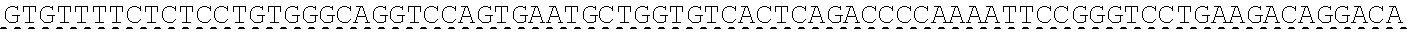

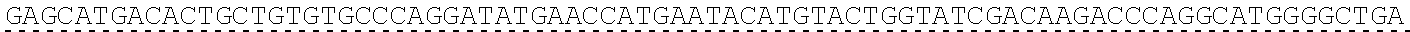

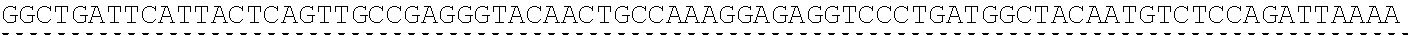

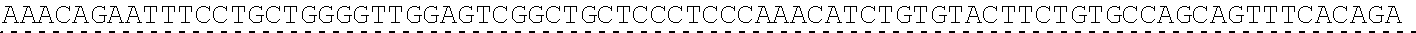

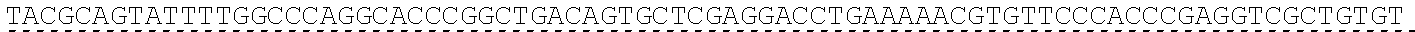

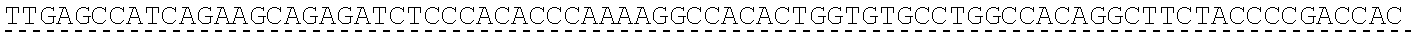

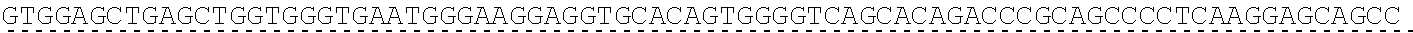

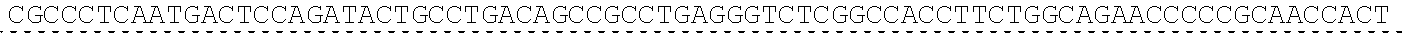

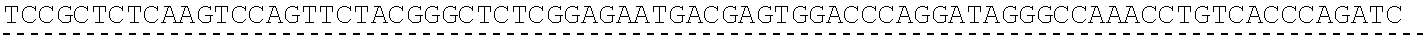

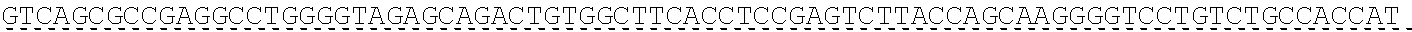

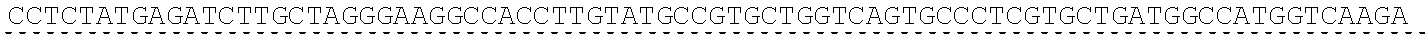

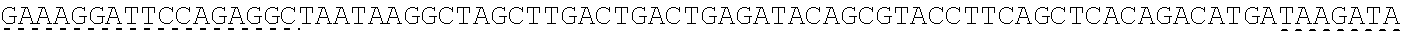

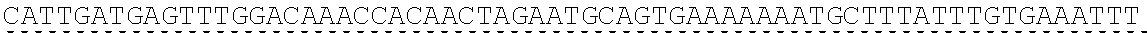

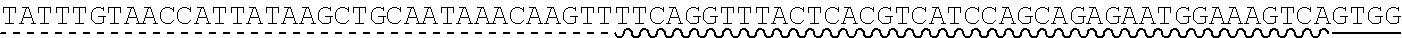

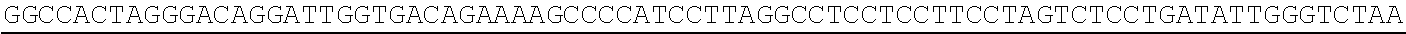

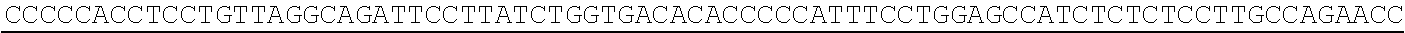

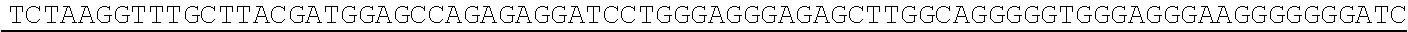

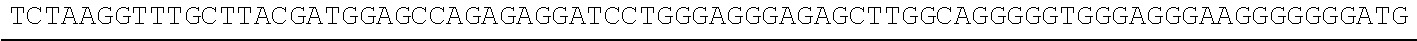

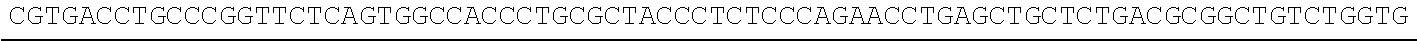

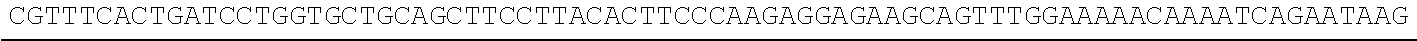

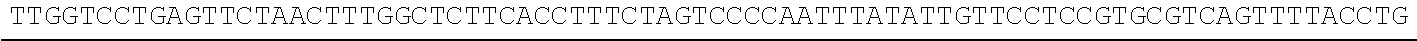

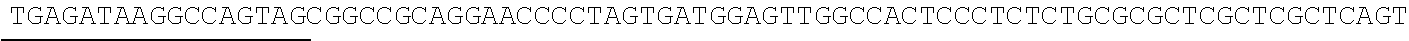

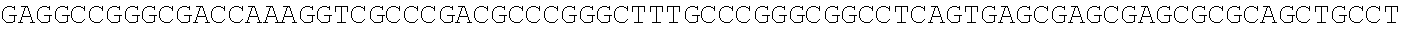

GAGGCCGGGCGACCAAAGGTCGCCCGACGCCCGGGCTTTGCCCGGGCGGCCTCAGTGAGCGAGCGAGCGCGCAGCTGCCT
GCAGGGGCGCCTGATGCGGTATTTTCTCCTTACGCATCTGTGCGGTATTTCACACCGCATACGTCAAAGCAACCATAGTA
CGCGCCCTGTAGCGGCGCATTAAGCGCGGCGGGTGTGGTGGTTACGCGCAGCGTGACCGCTACACTTGCCAGCGCCCTAG
CGCCCGCTCCTTTCGCTTTCTTCCCTTCCTTTCTCGCCACGTTCGCCGGCTTTCCCCGTCAAGCTCTAAATCGGGGGCTC
CCTTTAGGGTTCCGATTTAGTGCTTTACGGCACCTCGACCCCAAAAAACTTGATTTGGGTGATGGTTCACGTAGTGGGCC
ATCGCCCTGATAGACGGTTTTTCGCCCTTTGACGTTGGAGTCCACGTTCTTTAATAGTGGACTCTTGTTCCAAACTGGAA
CAACACTCAACTCTATCTCGGGCTATTCTTTTGATTTATAAGGGATTTTGCCGATTTCGGTCTATTGGTTAAAAAATGAG
CTGATTTAACAAAAATTTAACGCGAATTTTAACAAAATATTAACGTTTACAATTTTATGGTGCACTCTCAGTACAATCTG
CTCTGATGCCGCATAGTTAAGCCAGCCCCGACACCCGCCAACACCCGCTGACGCGCCCTGACGGGCTTGTCTGCTCCCGG
CATCCGCTTACAGACAAGCTGTGACCGTCTCCGGGAGCTGCATGTGTCAGAGGTTTTCACCGTCATCACCGAAACGCGCG
AGACGAAAGGGCCTCGTGATACGCCTATTTTTATAGGTTAATGTCATGATAATAATGGTTTCTTAGACGTCAGGTGGCAC
TTTTCGGGGAAATGTGCGCGGAACCCCTATTTGTTTATTTTTCTAAATACATTCAAATATGTATCCGCTCATGAGACAAT
AACCCTGATAAATGCTTCAATAATATTGAAAAAGGAAGAGTATGAGTATTCAACATTTCCGTGTCGCCCTTATTCCCTTT
TTTGCGGCATTTTGCCTTCCTGTTTTTGCTCACCCAGAAACGCTGGTGAAAGTAAAAGATGCTGAAGATCAGTTGGGTGC
ACGAGTGGGTTACATCGAACTGGATCTCAACAGCGGTAAGATCCTTGAGAGTTTTCGCCCCGAAGAACGTTTTCCAATGA
TGAGCACTTTTAAAGTTCTGCTATGTGGCGCGGTATTATCCCGTATTGACGCCGGGCAAGAGCAACTCGGTCGCCGCATA
CACTATTCTCAGAATGACTTGGTTGAGTACTCACCAGTCACAGAAAAGCATCTTACGGATGGCATGACAGTAAGAGAATT
ATGCAGTGCTGCCATAACCATGAGTGATAACACTGCGGCCAACTTACTTCTGACAACGATCGGAGGACCGAAGGAGCTAA
CCGCTTTTTTGCACAACATGGGGGATCATGTAACTCGCCTTGATCGTTGGGAACCGGAGCTGAATGAAGCCATACCAAAC
GACGAGCGTGACACCACGATGCCTGTAGCAATGGCAACAACGTTGCGCAAACTATTAACTGGCGAACTACTTACTCTAGC
TTCCCGGCAACAATTAATAGACTGGATGGAGGCGGATAAAGTTGCAGGACCACTTCTGCGCTCGGCCCTTCCGGCTGGCT
GGTTTATTGCTGATAAATCTGGAGCCGGTGAGCGTGGGTCTCGCGGTATCATTGCAGCACTGGGGCCAGATGGTAAGCCC
TCCCGTATCGTAGTTATCTACACGACGGGGAGTCAGGCAACTATGGATGAACGAAATAGACAGATCGCTGAGATAGGTGC
CTCACTGATTAAGCATTGGTAACTGTCAGACCAAGTTTACTCATATATACTTTAGATTGATTTAAAACTTCATTTTTAAT
TTAAAAGGATCTAGGTGAAGATCCTTTTTGATAATCTCATGACCAAAATCCCTTAACGTGAGTTTTCGTTCCACTGAGCG
TCAGACCCCGTAGAAAAGATCAAAGGATCTTCTTGAGATCCTTTTTTTCTGCGCGTAATCTGCTGCTTGCAAACAAAAAA
ACCACCGCTACCAGCGGTGGTTTGTTTGCCGGATCAAGAGCTACCAACTCTTTTTCCGAAGGTAACTGGCTTCAGCAGAG
CGCAGATACCAAATACTGTCCTTCTAGTGTAGCCGTAGTTAGGCCACCACTTCAAGAACTCTGTAGCACCGCCTACATAC
CTCGCTCTGCTAATCCTGTTACCAGTGGCTGCTGCCAGTGGCGATAAGTCGTGTCTTACCGGGTTGGACTCAAGACGATA
GTTACCGGATAAGGCGCAGCGGTCGGGCTGAACGGGGGGTTCGTGCACACAGCCCAGCTTGGAGCGAACGACCTACACCG
AACTGAGATACCTACAGCGTGAGCTATGAGAAAGCGCCACGCTTCCCGAAGGGAGAAAGGCGGACAGGTATCCGGTAAGC
GGCAGGGTCGGAACAGGAGAGCGCACGAGGGAGCTTCCAGGGGGAAACGCCTGGTATCTTTATAGTCCTGTCGGGTTTCG
CCACCTCTGACTTGAGCGTCGATTTTTGTGATGCTCGTCAGGGGGGCGGAGCCTATGGAAAAACGCCAGCAACGCGGCCT
TTTTACGGTTCCTGGCCTTTTGCTGGCCTTTTGCTCACATGT
SEQ ID NO: 59 (plasmid containing the A2M10 placeholder TCR cassette (ADB00794_001)
Full underline = homology arms; Wave underline = B2M target sites; Dashed underline =
A2M10 TCR Dotted underline = EF-1a promoter; Double underline = Furin cleavage site,
P2A; Dot-dash underline = SV40 polyA signal)
MMKSLRVLLVILWLQLSWVWSQQKEVEQNSGPLSVPEGAIASLNCTYSDRGSQSFFWYRQYSGKSPELIMSIYSNGDKED
GRFTAQLNKASQYVSLLIRDSQPSDSATYLCAVRGTGRRALTFGSGTRLQVQPNIQNPDPAVYQLRDSKSSDKSVCLFTD
FDSQTNVSQSKDSDVYITDKTVLDMRSMDFKSNSAVAWSNKSDFACANAFNNSIIPEDTFFPSPESSCDVKLVEKSFETD
TNLNFQNLSVIGFRILLLKVAGFNLLMTLRLWSSGSRAKRSGSGATNFSLLKQAGDVEENPGPRMSLGLLCCGVFSLLWA
GPVNAGVTQTPKFRVLKTGQSMTLLCAQDMNHEYMYWYRQDPGMGLRLIHYSVAEGTTAKGEVPDGYNVSRLKKQNFLLG
LESAAPSQTSVYFCASSFTDTQYFGPGTRLTVLEDLKNVFPPEVAVFEPSEAEISHTQKATLVCLATGFYPDHVELSWWV
NGKEVHSGVSTDPQPLKEQPALNDSRYCLSSRLRVSATFWQNPRNHFRCQVQFYGLSENDEWTQDRAKPVTQIVSAEAWG
RADCGFTSESYQQGVLSATILYEILLGKATLYAVLVSALVLMAMVKRKDSRG
SEQ ID NO: 60 (protein sequence of A2M10 placeholder TCR)

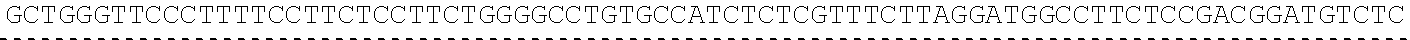

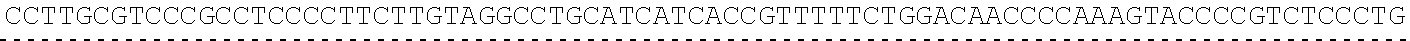

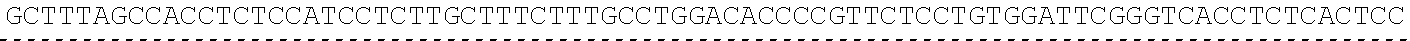

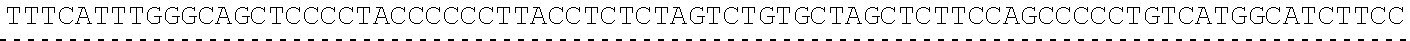

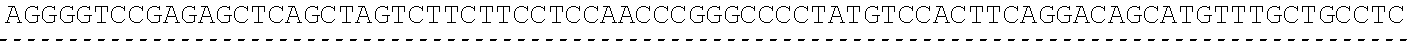

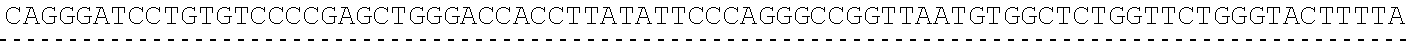

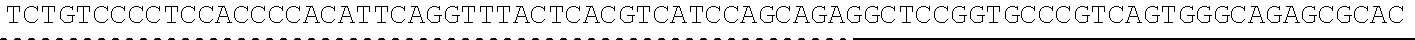

ATCGCCCACAGTCCCCGAGAAGITGGGGGGAGGGGTCGGCAATTGAACCGGTGCCTAGAGAAGGTGGCGCGGGGTAAACT
GGGAAAGTGATGTCGTGTACTGGCTCCGCCTTTTTCCCGAGGGTGGGGGAGAACCGTATATAAGTGCACTAGTCGCCGTG
AACGTTCTTTTTCGCAACGGGTTTGCCGCCAGAACACAGGTAAGTGCCGTGTGTGGTTCCCGCGGGCCTGGCCTCTTTAC
GGGTTATGGCCCTTGCGTGCCTTGAATTACTTCCACCTGGCTGCAGTACGTGATTCTTGATCCCGAGCTTCGGGTTGGAA
GTGGGTGGGAGAGTTCGTGGCCTTGCGCTTAAGGAGCCCCTTCGCCTCGTGCTTGAGTTCGTGGCCTGGCCTGGCGCTGC
GGCCGCCGCGTGCGAATCTGGTGGCACCTTCGCGCCTGTCTCGCTGCTTTCGATAAGTCTCTAGCCATTTAAAATTTTTG
ATGACCTGCTGCGACGCTTTTTTTCTGCGCAAGATAGTCTTGTAAATGCGGGCCAAGATCAGCACACTGGTATTCGGTTT
TTGGGGCCGCGGCGGCGACCGGGCGCCCGTGCCTCCCAGCGCACATGTTCGGCGAGGCGGCCTGCGAGCGCGGCCACCCG
AGAATCGGACGGGGGTAGTCTCAAGCTGCCCGGCCTGCTCTGGTGCCTGGCCTCGCGCCGCCGTGTATCGCCCCGCCCTG
GGCGGCAAGGCTGGCCCGGTCGGCACCAGTTGCGTGACGCGGAAAGATGGCCGCTTCCCGGCCCTGCTGCAGGAGCACAA
AATGGAGGACGCGGCGCTCGGAGAGCGGGCGCGCGTGAGTCACCCACACAAAGGAAAGGGCCTTTCCGTCCTCAGCCGTC
GCTTCATGTGACTCCACGGAGTACCGGGCGCCGTCCAGGCACCTCGATTAGTTCTCCAGCTTTGGAGTACCGTCGTCTTT
AGGTTGGGGGGAGGGGTTTATGCGATGGAGTTCCCCACACTGACTGCGCGTGGACACTGAAGTTAGGCCAGCTTGGCACT
TGATGTAATTCTCCTTGGAATTTGCCCTTTTTGAGTTTGGATCTTGGTTCATTCTCAAGCCTCAGACAGTGGTTCAAAGT
TTTTTTCTTCCATTTCAGGTCGTCGTGAAAACTACCCCTAAAGCCAAAAGATCTTTGTCGATCCTACCATCCACTCGACA
CACCCGCCAGCGGCCGCTGCCAAGCTTGCCGCCACCATCAACAAGCACCTGACCACCTTTCTCGTGATCCTGTGGCTGTA

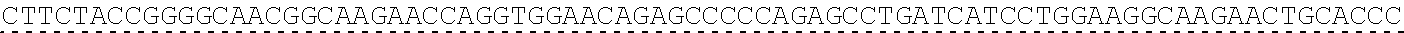

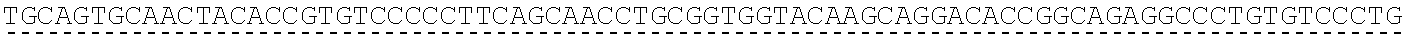

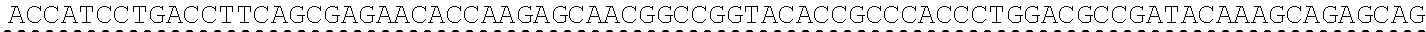

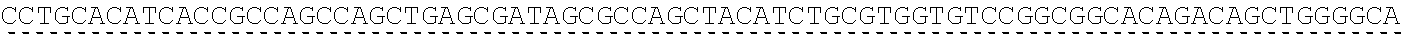

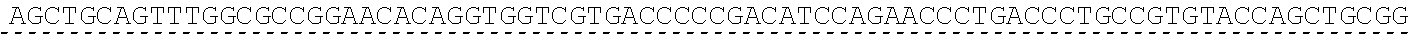

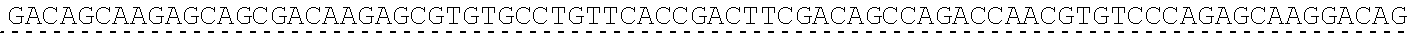

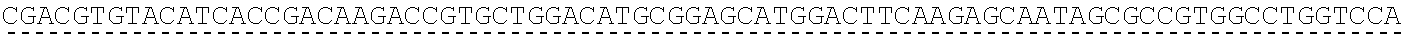

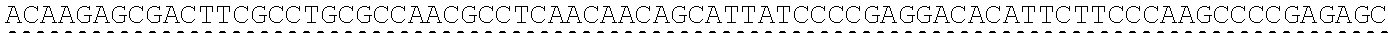

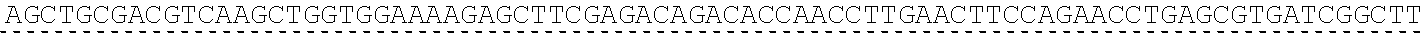

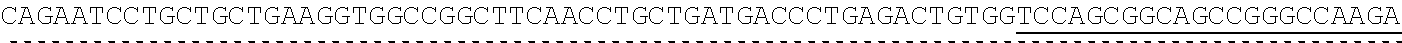

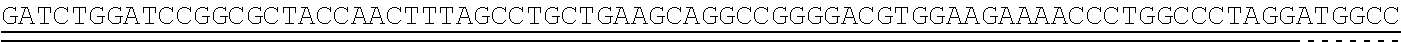

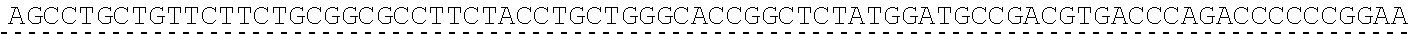

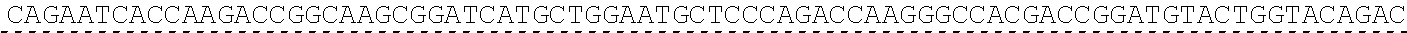

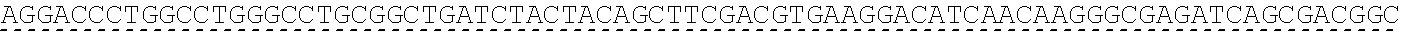

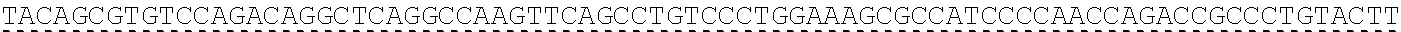

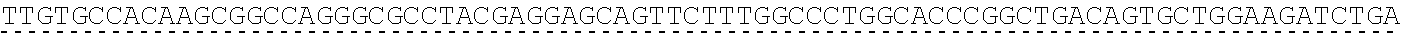

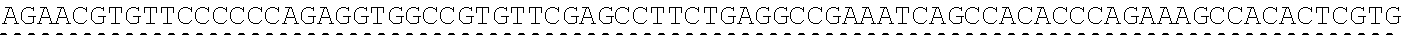

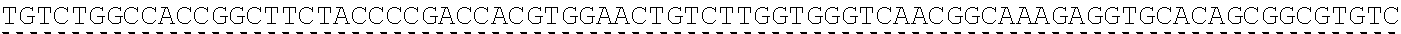

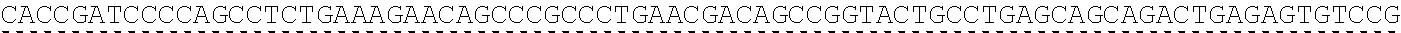

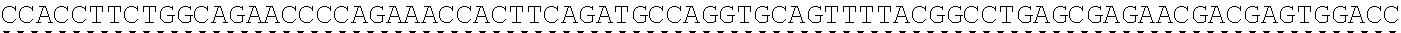

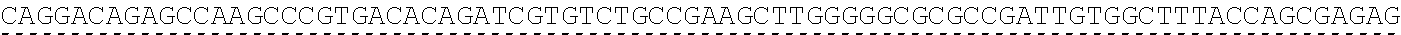

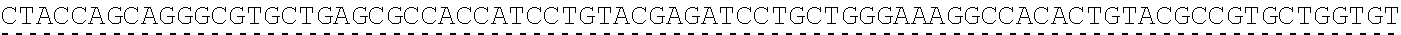

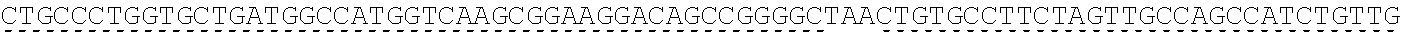

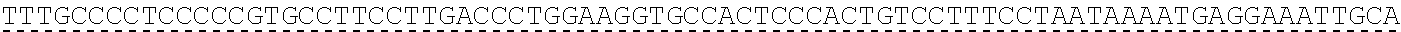

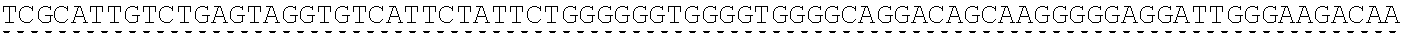

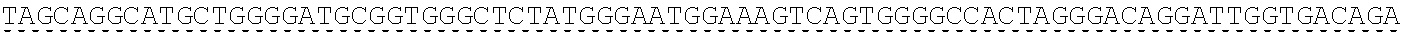

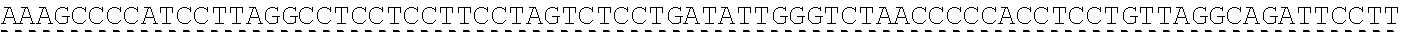

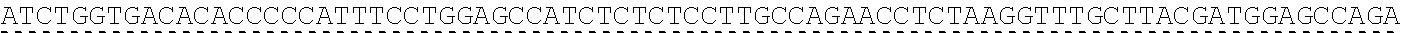

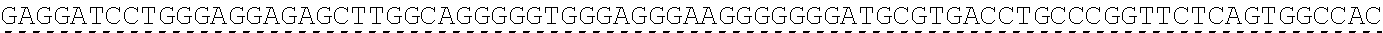

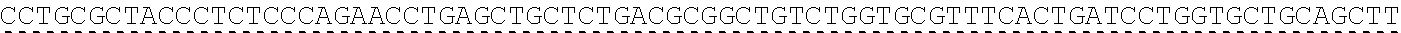

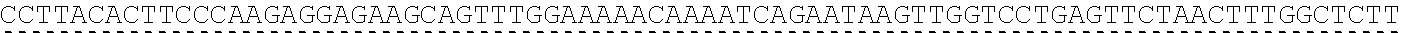

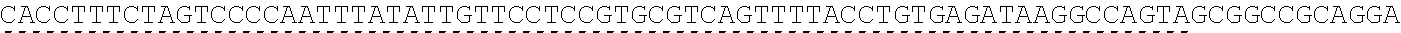

ACCCCTAGTGATGGAGTTGGCCACTCCCTCTCTGCGCGCTCGCTCGCTCACTGAGGCGGGCGACCAAAGGTCGCCCGAC
GCCCGGGCTTTGCCCGGGCGGCCTCAGTGAGCGAGCGAGCGCGCAGCTGCCTGCAGGGGCGCCTGATGCGGTATTTTCTC
CTTACGCATCTGTGCGGTATTTCACACCGCATACGTCAAAGCAACCATAGTACGCGCCCTGTAGCGGCGCATTAAGCGCG
GCGGGTGTGGTGGTTACGCGCAGCGTGACCGCTACACTTGCCAGCGCCCTAGCGCCCGCTCCTTTCGCTTTCTTCCCTTC
CTTTCTCGCCACGTTCGCCGGCTTTCCCCGTCAAGCTCTAAATCGGGGGCTCCCTTTAGGGTTCCGATTTAGTGCTTTAC
GGCACCTCGACCCCAAAAAACTTGATTTGGGTGATGGTTCACGTAGTGGGCCATCGCCCTGATAGACGGTTTTTCGCCCT
TTGACGTTGGAGTCCACGTTCTTTAATAGTGGACTCTTGTTCCAAACTGGAACAACACTCAACTCTATCTCGGGCTATTC
TTTTGATTTATAAGGGATTTTGCCGATTTCGGTCTATTGGTTAAAAAATGAGCTGATTTAACAAAAATTTAACGCGAATT
TTAACAAAATATTAACGTTTACAATTTTATGGTGCACTCTCAGTACAATCTGCTCTGATGCCGCATAGTTAAGCCAGCCC
CGACACCCGCCAACACCCGCTGACGCGCCCTGACGGGCTTGTCTGCTCCCGGCATCCGCTTACAGACAAGCTGTGACCGT
CTCCGGGAGCTGCATGTGTCAGAGGTTTTCACCGTCATCACCGAAACGCGCGAGACGAAAGGGCCTCGTGATACGCCTAT
TTTTATAGGTTAATGTCATGATAATAATGGTTTCTTAGACGTCAGGTGGCACTTTTCGGGGAAATGTGCGCGGAACCCCT
ATTTGTTTATTTTTCTAAATACATTCAAATATGTATCCGCTCATGAGACAATAACCCTGATAAATGCTTCAATAATATTG
AAAAAGGAAGAGTATGAGTATTCAACATTTCCGTGTCGCCCTTATTCCCTTTTTTGCGGCATTTTGCCTTCCTGTTTTTG
CTCACCCAGAAACGCTGGTGAAAGTAAAAGATGCTGAAGATCAGTTGGGTGCACGAGTGGGTTACATCGAACTGGATCTC
AACAGCGGTAAGATCCTTGAGAGTTTTCGCCCCGAAGAACGTTTTCCAATGATGAGCACTTTTAAAGTTCTGCTATGTGG
CGCGGTATTATCCCGTATTGACGCCGGGCAAGAGCAACTCGGTCGCCGCATACACTATTCTCAGAATGACTTGGTIGAGT
ACTCACCAGTCACAGAAAAGCATCTTACGGATGGCATGACAGTAAGAGAATTATGCAGTGCTGCCATAACCATGAGTGAT
AACACTGCGGCCAACTTACTTCTGACAACGATCGGAGGACCGAAGGAGCTAACCGCTTTTTTGCACAACATGGGGGATCA
TGTAACTCGCCTTGATCGTTGGGAACCGGAGCTGAATGAAGCCATACCAAACGACGAGCGTGACACCACGATGCCTGTAG
CAATGGCAACAACGTTGCGCAAACTATTAACTGGCGAACTACTTACTCTAGCTTCCCGGCAACAATTAATAGACTGGATG
GAGGCGGATAAAGTTGCAGGACCACTTCTGCGCTCGGCCCTTCCGGCTGGCTGGTTTATTGCTGATAAATCTGGAGCCGG
TGAGCGTGGGTCTCGCGGTATCATTGCAGCACTGGGGCCAGATGGTAAGCCCTCCCGTATCGTAGTTATCTACACGACGG
GGAGTCAGGCAACTATGGATGAACGAAATAGACAGATCGCTGAGATAGGTGCCTCACTGATTAAGCATTGGTAACTGTCA
GACCAAGTTTACTCATATATACTTTAGATTGATTTAAAACTTCATTTTTAATTTAAAAGGATCTAGGTGAAGATCCTTTT
TGATAATCTCATGACCAAAATCCCTTAACGTGAGTTTTCGTTCCACTGAGCGTCAGACCCCGTAGAAAAGATCAAAGGAT
CTTCTTGAGATCCTTTTTTTCTGCGCGTAATCTGCTGCTTGCAAACAAAAAAACCACCGCTACCAGCGGTGGTTTGTTTG
CCGGATCAAGAGCTACCAACTCTTTTTCCGAAGGTAACTGGCTTCAGCAGAGCGCAGATACCAAATACTGTCCTTCTAGT
GTAGCCGTAGTTAGGCCACCACTTCAAGAACTCTGTAGCACCGCCTACATACCTCGCTCTGCTAATCCTGTTACCAGTGG
CTGCTGCCAGTGGCGATAAGTCGTGTCTTACCGGGTTGGACTCAAGACGATAGTTACCGGATAAGGCGCAGCGGTCGGGC
TGAACGGGGGGTTCGTGCACACAGCCCAGCTTGGAGCGAACGACCTACACCGAACTGAGATACCTACAGCGTGAGCTATG
AGAAAGCGCCACGCTTCCCGAAGGGAGAAAGGCGGACAGGTATCCGGTAAGCGGCAGGGTCGGAACAGGAGAGCGCACGA
GGGAGCTTCCAGGGGGAAACGCCTGGTATCTTTATAGTCCTGTCGGGTTTCGCCACCTCTGACTTGAGCGTCGATTTTTG
TGATGCTCGTCAGGGGGGCGGAGCCTATGGAAAAACGCCAGCAACGCGGCCTTTTTACGGTTCCTGGCCTTTTGCTGGCC
TTTTGCTCACATGTCCTGCAGGCAGCTGCGCGCTCGCTCGCTCACTGAGGCCGCCCGGGCGTCGGGCGACCTTTGGTCGC
CCGGCCTCAGTGAGCGAGCGCGCAGAGAGGGAGTGGCCAACTCCATCACTAGGGGTTCCTGCGGCC
SEQ ID NO: 61 (plasmid containing the A2M4 exchange TCR cassette (ADB01032_026) Dotted
underline = homology arms; Full underline = EF-1x promoter; Dashed underline = A2M10 TCR;
Double underline = Furin cleavage site, P2A; Dot-dash underline = BGH polyA signal)
MKKHLTTFLVILWLYFYRGNGKNQVEQSPQSLIILEGKNCTLQCNYTVSPFSNLRWYKQDTGRGPVSLTILTFSENTKSN
GRYTATLDADTKQSSLHITASQLSDSASYICVVSGGTDSWGKLQFGAGTQVVVTPDIQNPDPAVYQLRDSKSSDKSVCLF
TDFDSQTNVSQSKDSDVYITDKTVLDMRSMDFKSNSAVAWSNKSDFACANAFNNSIIPEDTFFPSPESSCDVKLVEKSFE
TDTNLNFQNLSVIGFRILLLKVAGENLLMTLRLWSSGSRAKRSGSGATNFSLLKQAGDVEENPGPRMASLLFFCGAFYLL
GTGSMDADVTQTPRNRITKTGKRIMLECSQTKGHDRMYWYRQDPGLGLRLIYYSFDVKDINKGEISDGYSVSRQAQAKFS
LSLESAIPNQTALYFCATSGQGAYEEQFFGPGTRLTVLEDLKNVFPPEVAVFEPSEAEISHTQKATLVCLATGFYPDHVE
LSWWVNGKEVHSGVSTDPQPLKEQPALNDSRYCLSSRLRVSATFWQNPRNHFRCQVQFYGLSENDEWTQDRAKPVTQIVS
AEAWGRADCGFTSESYQQGVLSATILYEILLGKATLYAVLVSALVLMAMVKRKDSRG
SEQ ID NO: 62 (protein sequence of the A2M4 exchange TCR)
UCACGUCAUCCAGCAGAGAA
SEQ ID NO: 63 (CRISPR-Cas9 guide RNA)
CTGGGTTCCCTTTTCCTTCTCCTTCTGGGGCCTGTGCCATCTCTCGTTTCTTAGGATGGCCTTCTCCGACGGATGTCTCC
CTTGCGTCCCGCCTCCCCTTCTTGTAGGCCTGCATCATCACCGTTTTTCTGGACAACCCCAAAGTACCCCGTCTCCCTGG
CTTTAGCCACCTCTCCATCCTCTTGCTTTCTTTGCCTGGACACCCCGTTCTCCTGTGGATTCGGGTCACCTCTCACTCCT
TTCATTTGGGCAGCTCCCCTACCCCCCTTACCTCTCTAGTCTGTGCTAGCTCTTCCAGCCCCCTGTCATGGCATCTTCCA
GGGGTCCGAGAGCTCAGCTAGTCTTCTTCCTCCAACCCGGGCCCCTATGTCCACTTCAGGACAGCATGTTTGCTGCCTCC
AGGGATCCTGTGTCCCCGAGCTGGGACCACCTTATATTCCCAGGGCCGGTTAATGTGGCTCTGGTTCTGGGTACTTTTAT
CTGTCCCCTCCACCCCACA
SEQ ID NO: 64 - plasmid ADB00794_001 left homology arm (Chr 19: 55115774 to 55116272 Human
GRCh38. p14 Primary Assembly
GGCTCCGGTGCCCGTCAGTGGGCAGAGCGCACATCGCCCACAGTCCCCGAGAAGTTGGGGGGAGGGGTCGGCAATTGAAC
CGGTGCCTAGAGAAGGTGGCGCGGGGTAAACTGGGAAAGTGATGTCGTGTACTGGCTCCGCCTTTTTCCCGAGGGTGGGG
GAGAACCGTATATAAGTGCACTAGTCGCCGTGAACGTTCTTTTTCGCAACGGGTTTGCCGCCAGAACACAGGTAAGTGCC
GTGTGTGGTTCCCGCGGGCCTGGCCTCTTTACGGGTTATGGCCCTTGCGTGCCTTGAATTACTTCCACCTGGCTGCAGTA
CGTGATTCTTGATCCCGAGCTTCGGGTTGGAAGTGGGTGGGAGAGTTCGTGGCCTTGCGCTTAAGGAGCCCCTTCGCCTC
GTGCTTGAGTTGTGGCCTGGCCTGGGCGCTGGGGCCGCCGCGTGCGAATCTGGTGGCACCTTCGCGCCTGTCTCGCTGCT
TTCGATAAGTCTCTAGCCATTTAAAATTTTTGATGACCTGCTGCGACGCTTTTTTTCTGGCAAGATAGTCTTGTAAATGC
GGGCCAAGATCAGCACACTGGTATTTCGGTTTTTGGGGCCGCGGGCGGCGACGGGGCCCGTGCGTCCCAGCGCACATGTT
CGGCGAGGCGGGGCCTGCGAGCGCGGCCACCGAGAATCGGACGGGGGTAGTCTCAAGCTGCCCGGCCTGCTCTGGTGCCT
GGCCTCGCGCCGCCGTGTATCGCCCCGCCCTGGGCGGCAAGGCTGGCCCGGTCGGCACCAGTTGCGTGAGCGGAAAGATG
GCCGCTTCCCGGCCCTGCTGCAGGGAGCACAAAATGGAGGACGCGGCGCTCGGGAGAGCGGGCGGGTGAGTCACCCACAC
AAAGGAAAAGGGCCTTTCCGTCCTCAGCCGTCGCTTCATGTGACTCCACGGAGTACCGGGCGCCGTCCAGGCACCTCGAT
TAGTTCTCCAGCTTTTGGAGTACGTCGTCTTTAGGTTGGGGGGAGGGGTTTTATGCGATGGAGTTTCCCCACACTGAGTG
GGTGGAGACTGAAGTTAGGCCAGCTTGGCACTTGATGTAATTCTCCTTGGAATTTGCCCTTTTTGAGTTTGGATCTTGGT
TCATTCTCAAGCCTCAGACAGTGGTTCAAAGTTTTTTTCTTCCATTTCAGGTGTCGTGA
SEQ ID NO: 65-EF1 alpha promotor DNA sequence
ATGATGAAATCCTTGAGAGTTTTACTAGTGATCCTGTGGCTTCAGTTGAGCTGGGTTTGGAGCCAACAGAAGGAGGTGGA
GCAGAATTCTGGACCCCTCAGTGTTCCAGAGGGAGCCATTGCCTCTCTCAACTGCACTTACAGTGACCGAGGTTCCCAGT
CCTTCTTCTGGTACAGACAATATTCTGGGAAAAGCCCTGAGTTGATAATGTCCATATACTCCAATGGTGACAAAGAAGAT
GGAAGGTTTACAGCACAGCTCAATAAAGCCAGCCAGTATGTTTCTCTGCTCATCAGAGACTCCCAGCCCAGTGATTCAGC
CACCTACCTCTGTGCCGTGAGAGGCACGGGCAGGAGAGCACTTACTTTTGGGAGTGGAACAAGACTCCAAGTGCAACCAA
ATATCCAGAACCCTGACCCTGCCGTGTACCAGCTGAGAGACTCTAAATCCAGTGACAAGTCTGTCTGCCTATTCACCGAT
TTTGATTCTCAAACAAATGTGTCACAAAGTAAGGATTCTGATGTGTATATCACAGACAAAACTGTGCTAGACATGAGGTC
TATGGACTTCAAGAGCAACAGTGCTGTGGCCTGGAGCAACAAATCTGACTTTGCATGTGCAAACGCCTTCAACAACAGCA
TTATTCCAGAAGACACCTTCTTCCCCAGCCCAGAAAGTTCCTGTGATGTCAAGCTGGTCGAGAAAAGCTTTGAAACAGAT
ACGAACCTAAACTTTCAAAACCTGTCAGTGATTGGGTTCCGAATCCTCCTCCTGAAAGTGGCCGGGTTTAATCTGCTCAT
GACGCTGCGGCTGTGGTCCAGC
SEQ ID NO: 66-A2M10 c794 TCR alpha chain nucleotide sequence (from ADB00794_001)
MMKSLRVLLVILWLQLSWVWSQQKEVEQNSGPLSVPEGAIASLNCTYSDRGSQSFFWYRQYSGKSPELIMSIYSNGDKED
GRFTAQLNKASQYVSLLIRDSQPSDSATYLCAVRGTGRRALTFGSGTRLQVQPNIQNPDPAVYQLRDSKSSDKSVCLFTD
FDSQTNVSQSKDSDVYITDKTVLDMRSMDFKSNSAVAWSNKSDFACANAFNNSIIPEDTFFPSPESSCDVKLVEKSFETD
TNLNFQNLSVIGFRILLLKVAGFNLLMTLRLWSS
SEQ ID NO: 67-A2M10 c794 alpha chain amino acid sequence (from ADB00794_001)
GGCAGCCGGGCCAAGAGAAGCGGATCCGGC
SEQ ID NO: 68-Furin SG linker nucleotide sequence
GCCACCAACTTCAGCCTGCTGAAGCAGGCCGGCGACGTGGAGGAAAACCCTGGCCCTAGG
SEQ ID NO: 69-P2A skip like sequence nucleotide sequence
ATNFSLLKQAGDVEENPGPR
SEQ ID NO: 70-P2A skip like sequence amino acid sequence
ATGAGCCTCGGGCTCCTGTGCTGTGGGGTGTTTTCTCTCCTGTGGGCAGGTCCAGTGAATGCTGGTGTCACTCAGACCCC
AAAATTCCGGGTCCTGAAGACAGGACAGAGCATGACACTGCTGTGTGCCCAGGATATGAACCATGAATACATGTACTGGT
ATCGACAAGACCCAGGCATGGGGCTGAGGCTGATTCATTACTCAGTTGCCGAGGGTACAACTGCCAAAGGAGAGGTCCCT
GATGGCTACAATGTCTCCAGATTAAAAAAACAGAATTTCCTGCTGGGGTTGGAGTCGGCTGCTCCCTCCCAAACATCTGT
GTACTTCTGTGCCAGCAGTTTCACAGATACGCAGTATTTTGGCCCAGGCACCCGGCTGACAGTGCTCGAGGACCTGAAAA
ACGTGTTCCCACCCGAGGTCGCTGTGTTTGAGCCATCAGAAGCAGAGATCTCCCACACCCAAAAGGCCACACTGGTGTGC
CTGGCCACAGGCTTCTACCCCGACCACGTGGAGCTGAGCTGGTGGGTGAATGGGAAGGAGGTGCACAGTGGGGTCAGCAC
AGACCCGCAGCCCCTCAAGGAGCAGCCCGCCCTCAATGACTCCAGATACTGCCTGAGCAGCCGCCTGAGGGTCTCGGCCA
CCTTCTGGCAGAACCCCCGCAACCACTTCCGCTGTCAAGTCCAGTTCTACGGGCTCTCGGAGAATGACGAGTGGACCCAG
GATAGGGCCAAACCTGTCACCCAGATCGTCAGCGCCGAGGCCTGGGGTAGAGCAGACTGTGGCTTCACCTCCGAGTCTTA
CCAGCAAGGGGTCCTGTCTGCCACCATCCTCTATGAGATCTTGCTAGGGAAGGCCACCTTGTATGCCGTGCTGGTCAGTG
CCCTCGTGCTGATGGCCATGGTCAAGAGAAAGGATTCCAGAGGC
SEQ ID NO: 71-A2M10 c794 TCR beta chain nucleotide sequence (from ADB00794_001)
MSLGLLCCGVFSLLWAGPVNAGVTQTPKFRVLKTGQSMTLLCAQDMNHEYMYWYRQDPGMGLRLIHYSVAEGTTAKGEVP
DGYNVSRLKKQNFLLGLESAAPSQTSVYFCASSFTDTQYFGPGTRLTVLEDLKNVFPPEVAVFEPSEAEISHTQKATLVC
LATGFYPDHVELSWWVNGKEVHSGVSTDPQPLKEQPALNDSRYCLSSRLRVSATFWQNPRNHFRCQVQFYGLSENDEWTQ
DRAKPVTQIVSAEAWGRADCGFTSESYQQGVLSATILYEILLGKATLYAVLVSALVLMAMVKRKDSRG
SEQ ID NO: 72-A2M10 c794 TCR beta chain amino acid sequence (from ADB00794_001)
TAAGATACATTGATGAGTTTGGACAAACCACAACTAGAATGCAGTGAAAAAAATGCTTTATTTGTGAAATTTGTGATGCT
ATTGCTTTATTTGTAACCATTATAAGCTGCAATAAACAAGTT
SEQ ID NO: 73-SV40 polyA signal nucleotide sequence
GTGGGGCCACTAGGGACAGGATTGGTGACAGAAAAGCCCCATCCTTAGGCCTCCTCCTTCCTAGTCTCCTGATATTGGGT
CTAACCCCCACCTCCTGTTAGGCAGATTCCTTATCTGGTGACACACCCCCATTTCCTGGAGCCATCTCTCTCCTTGCCAG
AACCTCTAAGGTTTGCTTACGATGGAGCCAGAGAGGATCCTGGGAGGGAGAGCTTGGCAGGGGGTGGGAGGGAAGGGGGG
GATGCGTGACCTGCCCGGTTCTCAGTGGCCACCCTGCGCTACCCTCTCCCAGAACCTGAGCTGCTCTGACGCGGCTGTCT
GGTGCGTTTCACTGATCCTGGTGCTGCAGCTTCCTTACACTTCCCAAGAGGAGAAGCAGTTTGGAAAAACAAAATCAGAA
TAAGTTGGTCCTGAGTTCTAACTTTGGCTCTTCACCTTTCTAGTCCCCAATTTATATTGTTCCTCCGTGCGTCAGTTTTA
CCTGTGAGATAAGGCCAGTA
SEQ ID NO: 74-plasmid ADB00794_001 right homology arm (Chr 19: 55115274 to 55115773 Human
GRCh38. p14 Primary Assembly)
GACTTTCCATTCTCTGCTGGATGACGTGAGTAAACCTGAATGTGGGGTGGAGGGGACAG
SEQ ID NO: 75-reverse primer for amplification of the strategy 1 plasmid ADB00794_001
left homology arm (binding Chr 19: 55115774-55115792 GRCh38.p14).
Underlined-B2M sgRNA target site.
GGCTCCGGTGCCCGTCAGTGGGCAGAGCGCACATCGCCCACAGTCCCCGAGAAGTTGGGGGGAGGGGTCGGCAATTGAAC
CGGTGCCTAGAGAAGGTGGCGCGGGGTAAACTGGGAAAGTGATGTCGTGTACTGGCTCCGCCTTTTTCCCGAGGGTGGGG
GAGAACCGTATATAAGTGCACTAGTCGCCGTGAACGTTCTTTTTCGCAACGGGTTTGCCGCCAGAACACAGGTAAGTGCC
GTGTGTGGTTCCCGCGGGCCTGGCCTCTTTACGGGTTATGGCCCTTGCGTGCCTTGAATTACTTCCACCTGGCTGCAGTA
CGTGATTCTTGATCCCGAGCTTCGGGTTGGAAGTGGGTGGGAGAGTTCGTGGCCTTGCGCTTAAGGAGCCCCTTCG
ACTGGGTTCCCTTTTCCTTCTCCTTCTGGGGCCTGTGCCATCTCTCGTTTCTTAGGATGGCCTTCTCCGACGGATGTCTC
CCTTGCGTCCCGCCTCCCCTTCTTGTAGGCCTGCATCATCACCGTTTTTCTGGACAACCCCAAAGTACCCCGTCTCCCTG
GCTTTAGCCACCTCTCCATCCTCTTGCTTTCTTTGCCTGGACACCCCGTTCTCCTGTGGATTCGGGTCACCTCTCACTCC
TTTCATTTGGGCAGCTCCCCTACCCCCCTTACCTCTCTAGTCTGTGCTAGCTCTTCCAGCCCCCTGTCATGGCATCTTCC
AGGGTCCGAGAGCTCAGCTAGTCTTCTTCCTCCAACCCGGGCCCCTATGTCCACTTCAGCGACAGCATGTTTGCTGCCTC
CAGGGATCCTGTGTCCCCGAGCTGGGACCACCTTATATTCCCAGGGCCGGTTAATGTGGCTCTGGTTCTGGGTACTTTTA
TCTGTCCCCTCCACCCCACACCTCGTGCTTGAGTIGTGGCCTGGCCTGGGCGCTGGGGCCGCCGCGTGCGAATCTGGTGG
CACCTTCGCGCCTGTCTCGCTGCTTTCGATAAGTCTCTAGCCATTTAAAATTTTTGATGACCTGCTGCGACGCTTTTTTT
CTGGCAAGATAGTCTTGTAAATGCGGGCCAAGATCAGCACACTGGTATTTCGGTTTTTGGGGCCGCGGGCGGCGACGGGG
CCCGTGCGTCCCAGCGCACATGTTCGGCGAGGCGGGGCCTGCGAGCGCGGCCACCGAGAATCGGACGGGGGTAGTCTCAC
GCTGCCCGGCCTGCTCTGGTGCCTGCGCCTCGCGCCGCCGTGTATCGCCCGCCCTGGGCCGCAAGGCTGGCCCGGTCGGC
ACCAGTTGCGTGAGCGGAAAGATGGCCGCTTCCCGGCCCTGCTGCAGCCAGCACAAAATGGAGGACGCGGCGCTCGGGAG
AGCGGGCGGGTGAGTCACCCACACAAAGGAAAAGGGCCTTTCCGTCCTCAGCCGTCGCTTCATGTGACTCCACGGAGTAC
CGGGCGCCGTCCAGGCACCTCGATTAGTTCTCCAGCTTGGAGTACGTCGTCTTTAGGTTCGGGGCGAGGGCGTTTTATGC
GATGGAGTTTCCCCACACTGAGTGGGTGGAGACTGAAGTTAGGCCAGCTTGGCACTTGATGTAATTCTCCTTGGAATTTG
CCCTTTTTGAGTTTGGATCTTGGTTCATTCTCAAGCCTCAGACAGTGGTTCAAAGTTTTTTTCTTCCATTTCAGGTGTCG
TGAAAACTACCCCTAAAAGCCAAAAGATCTTTGTCGATCCTACCATCCACTCGACACACCCGCCAGCGGCCGCTGCCAAG

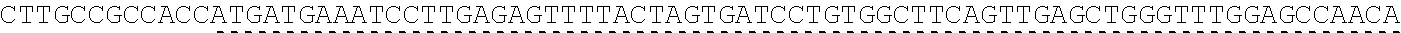

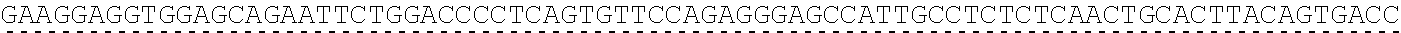

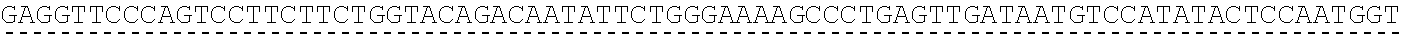

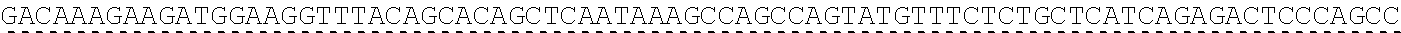

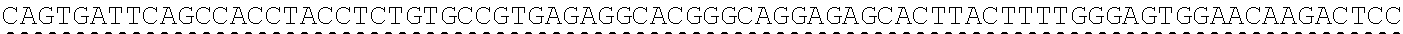

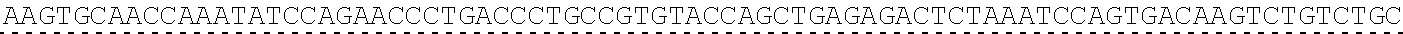

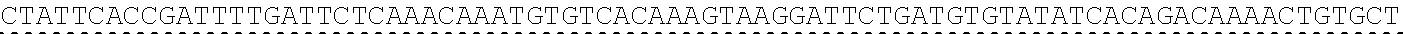

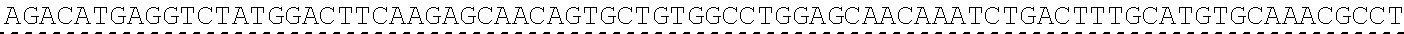

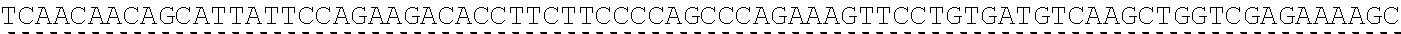

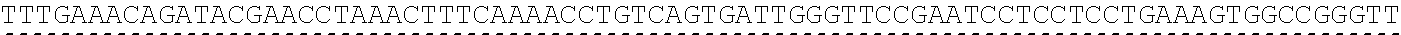

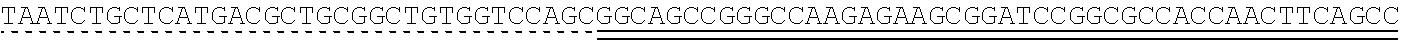

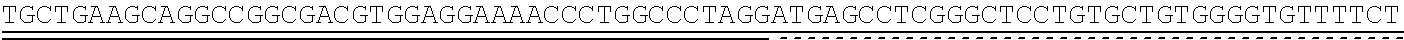

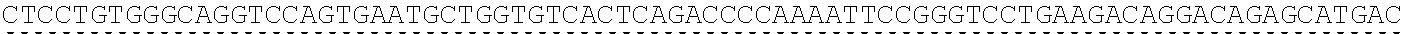

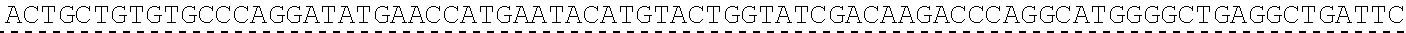

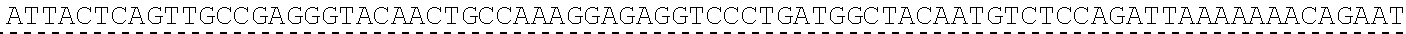

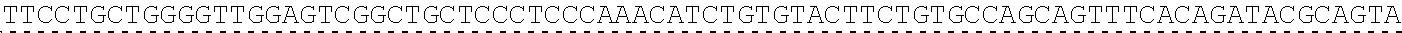

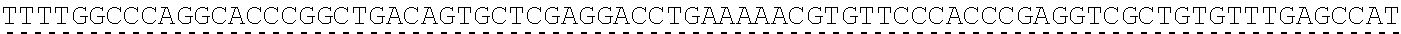

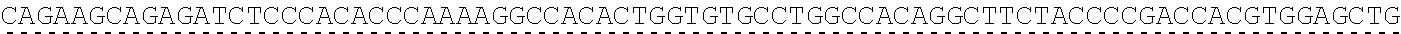

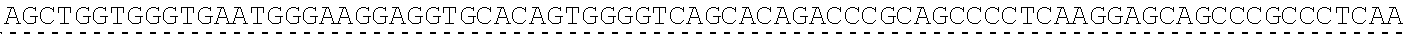

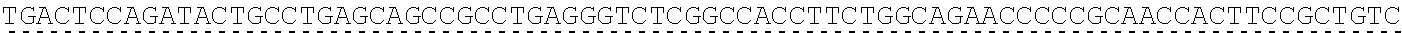

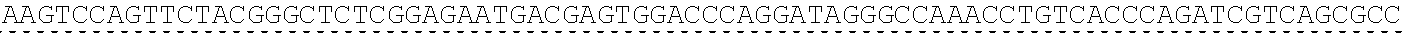

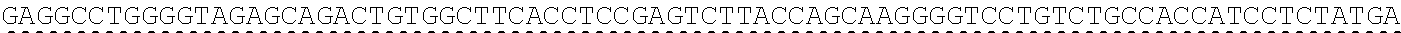

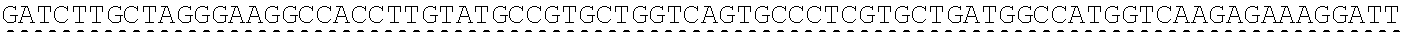

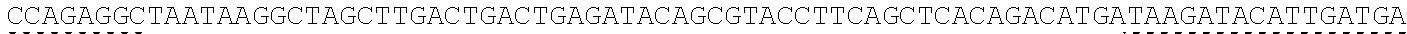

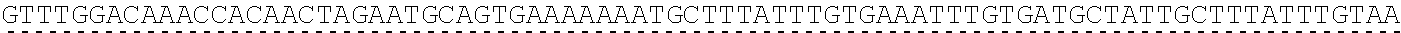

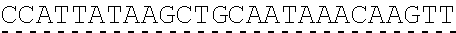

SEQ ID NO: 76-nucleotide sequence of the A2M10 TCR cassette. Underline = EF-1a promoter.
Dotted underline = the SV40 polyadenylation signal. Dashed underline = A2M10 TCR.
Double underline = Furin cleavage site, P2A
GCTGGGTTCCCTTTTCCTTCTCCTTCTGGGGCCTGTGCCATCTCTCGTTTCTTAGGATGGCCTTCTCCGACGGATGTCTC
CCTTGCGTCCCGCCTCCCCTTCTTGTAGGCCTGCATCATCACCGTTTTTCTGGACAACCCCAAAGTACCCCGTCTCCCTG
GCTTTAGCCACCTCTCCATCCTCTTGCTTTCTTTGCCTGGACACCCCGTTCTCCTGTGGATTCGGGTCACCTCTCACTCC
TTTCATTTGGGCAGCTCCCCTACCCCCCTTACCTCTCTAGTCTGTGCTAGCTCTTCCAGCCCCCTGTCATGGCATCTTCC
AGGGGTCCGAGAGCTCAGCTAGTCTTCTTCCTCCAACCCGGGCCCCTATGTCCACTTCAGGACAGCATGTTTGCTGCCTC
CAGGGATCCTGTGTCCCCGAGCTGGGACCACCTTATATTCCCAGGGCCGGTTAATGTGGCTCTGGTTCTGGGTACTTTTA
TCTGTCCCCTCCACCCCACATTCAGGTTTACTCACGTCATCCAGCAGA
SEQ ID NO: 77-plasmid ADB01032_026 left homology arm (Human Chr19: 55115774 to 55116273,
Chr15: 44715434 to 44715462 GRCh38. p14 Primary Assembly)
ATGAAGAAGCACCTGACCACCTTTCTCGTGATCCTGTGGCTGTACTTCTACCGGGGCAACGGCAAGAACCAGGTGGAACA
GAGCCCCCAGAGCCTGATCATCCTGGAAGGCAAGAACTGCACCCTGCAGTGCAACTACACCGTGTCCCCCTTCAGCAACC
TGCGGTGGTACAAGCAGGACACCGGCAGAGGCCCTGTGTCCCTGACCATCCTGACCTTCAGCGAGAACACCAAGAGCAAC
GGCCGGTACACCGCCACCCTGGACGCCGATACAAAGCAGAGCAGCCTGCACATCACCGCCAGCCAGCTGAGCGATAGCGC
CAGCTACATCTGCGTGGTGTCCGGCGGCACAGACAGCTGGGGCAAGCTGCAGTTTGGCGCCGGAACACAGGTGGTCGTGA
CCCCCGACATCCAGAACCCTGACCCTGCCGTGTACCAGCTGCGGGACAGCAAGAGCAGCGACAAGAGCGTGTGCCTGTTC
ACCGACTTCGACAGCCAGACCAACGTGTCCCAGAGCAAGGACAGCGACGTGTACATCACCGACAAGACCGTGCTGGACAT
GCGGAGCATGGACTTCAAGAGCAATAGCGCCGTGGCCTGGTCCAACAAGAGCGACTTCGCCTGCGCCAACGCCTTCAACA
ACAGCATTATCCCCGAGGACACATTCTTCCCAAGCCCCGAGAGCAGCTGCGACGTCAAGCTGGTGGAAAAGAGCTTCGAG
ACAGACACCAACCTGAACTTCCAGAACCTGAGCGTGATCGGCTTCAGAATCCTGCTGCTGAAGGTGGCCGGCTTCAACCT
GCTGATGACCCTGAGACTGTGG
SEQ ID NO: 78-A2M4 c1032 TCR alpha chain nucleotide sequence (from ADB01032_026)
MKKHLTTFLVILWLYFYRGNGKNQVEQSPQSLIILEGKNCTLQCNYTVSPFSNLRWYKQDTGRGPVSLTILTFSENTKSN
GRYTATLDADTKQSSLHITASQLSDSASYICVVSGGTDSWGKLQFGAGTQVVVTPDIQNPDPAVYQLRDSKSSDKSVCLF
TDFDSQTNVSQSKDSDVYITDKTVLDMRSMDFKSNSAVAWSNKSDFACANAFNNSIIPEDTFFPSPESSCDVKLVEKSFE
TDTNLNFQNLSVIGFRILLLKVAGENLLMTLRLW
SEQ ID NO: 79-A2M4 c1032 TCR alpha chain amino acid sequence (from ADB01032_026)
GCTACCAACTTTAGCCTGCTGAAGCAGGCCGGGGACGTGGAAGAAAACCCTGGCCCTAGG
SEQ ID NO: 80-P2A skip like sequence nucleotide sequence
ATGGCCAGCCTGCTGTTCTTCTGCGGCGCCTTCTACCTGCTGGGCACCGGCTCTATGGATGCCGACGTGACCCAGACCCC
CCGGAACAGAATCACCAAGACCGGCAAGCGGATCATGCTGGAATGCTCCCAGACCAAGGGCCACGACCGGATGTACTGGT
ACAGACAGGACCCTGGCCTGGGCCTGCGGCTGATCTACTACAGCTTCGACGTGAAGGACATCAACAAGGGCGAGATCAGC
GACGGCTACAGCGTGTCCAGACAGGCTCAGGCCAAGTTCAGCCTGTCCCTGGAAAGCGCCATCCCCAACCAGACCGCCCT
GTACTTTTGTGCCACAAGCGGCCAGGGCGCCTACGAGGAGCAGTTCTTTGGCCCTGGCACCCGGCTGACAGTGCTGGAAG
ATCTGAAGAACGTGTTCCCCCCAGAGGTGGCCGTGTTCGAGCCTTCTGAGGCCGAAATCAGCCACACCCAGAAAGCCACA
CTCGTGTGTCTGGCCACCGGCTTCTACCCCGACCACGTGGAACTGTCTTGGTGGGTCAACGGCAAAGAGGTGCACAGCGG
CGTGTCCACCGATCCCCAGCCTCTGAAAGAACAGCCCGCCCTGAACGACAGCCGGTACTGCCTGAGCAGCAGACTGAGAG
TGTCCGCCACCTTCTGGCAGAACCCCAGAAACCACTTCAGATGCCAGGTGCAGTTTTACGGCCTGAGCGAGAACGACGAG
TGGACCCAGGACAGAGCCAAGCCCGTGACACAGATCGTGTCTGCCGAAGCTTGGGGGCGCGCCGATTGTGGCTTTACCAG
CGAGAGCTACCAGCAGGGCGTGCTGAGCGCCACCATCCTGTACGAGATCCTGCTGGGAAAGGCCACACTGTACGCCGTGC
TGGTGTCTGCCCTGGTGCTGATGGCCATGGTCAAGCGGAAGGACAGCCGGGGCTAA
SEQ ID NO: 81-A2M4 c1032 TCR beta chain nucleotide sequence (from ADB01032_026)
MASLLFFCGAFYLLGTGSMDADVTQTPRNRITKTGKRIMLECSQTKGHDRMYWYRQDPGLGLRLIYYSEDVKDINKGEIS
DGYSVSRQAQAKFSLSLESAIPNQTALYFCATSGQGAYEEQFFGPGTRLTVLEDLKNVFPPEVAVFEPSEAEISHTQKAT
LVCLATGFYPDHVELSWWVNGKEVHSGVSTDPQPLKEQPALNDSRYCLSSRLRVSATFWQNPRNHERCOVOFYGLSENDE
WTQDRAKPVTQIVSAEAWGRADCGFTSESYQQGVLSATILYEILLGKATLYAVLVSALVLMAMVKRKDSRG
SEQ ID NO: 82-A2M4 c1032 TCR beta chain amino acid sequence (from ADB01032_026)
GAATGGAAAGTCAGTGGGGCCACTAGGGACAGGATTGGTGACAGAAAAGCCCCATCCTTAGGCCTCCTCCTTCCTAGTCT
CCTGATATTGGGTCTAACCCCCACCTCCTGTTAGGCAGATTCCTTATCTGGTGACACACCCCCATTTCCTGGAGCCATCT
CTCTCCTTGCCAGAACCTCTAAGGTTTGCTTACGATGGAGCCAGAGAGGATCCTGGGAGGGAGAGCTTGGCAGGGGGTGG
GAGGGAAGGGGGGGATGCGTGACCTGCCCGGTTCTCAGTGGCCACCCTGCGCTACCCTCTCCCAGAACCTGAGCTGCTCT
GACGCGGCTGTCTGGTGCGTTTCACTGATCCTGGTGCTGCAGCTTCCTTACACTTCCCAAGAGGAGAAGCAGTTTGGAAA
AACAAAATCAGAATAAGTTGGTCCTGAGTTCTAACTTTGGCTCTTCACCTTTCTAGTCCCCAATTTATATTGTTCCTCCG
TGCGTCAGTTTTACCTGTGAGATAAGGCCAGTA
SEQ ID NO: 83-plasmid ADB01032_026 right homology arm (Human Chr19: 55115274 to 55115775
GRCh38. p14 Primary Assembly)
GGCTCCGGTGCCCGTCAGTGGGC
SEQ ID NO: 84-forward primer for amplification of the EF1 alpha promoter
GGTGGCGGCAAGCTTGGCAGCGGC
SEQ ID NO: 85-reverse primer for amplification of the EF1 alpha promoter

1. A method for producing an immune cell expressing a therapeutic antigen receptor comprising;
(i) providing an immune cell comprising a heterologous expression cassette,
wherein the expression cassette comprises;
(a) a coding sequence for a production T cell receptor (TCR),
(b) a constitutive promoter operably linked to the coding sequence,
(c) a targeting site, and
(ii) replacing the heterologous expression cassette in the immune cell with an expression construct comprising a coding sequence for a therapeutic antigen receptor,
such that the therapeutic antigen receptor is expressed in the immune cell.

2. A method according to claim 1 wherein the therapeutic antigen receptor binds specifically to an MHC displaying a peptide fragment of a target antigen expressed by cells or specifically binds to a target antigen or peptide thereof expressed by cells independently of MHC presentation.

3. A method according to claim 2 wherein the therapeutic antigen receptor binds specifically to an MHC displaying a peptide fragment of a tumour antigen expressed by cancer cells or binds specifically to a tumour antigen or peptide fragment thereof expressed by cancer cells independently of MHC presentation.

4. A method according to any one of the preceding claims wherein the immune cell is a T cell.

5. A method according to any one of the preceding claims wherein the heterologous expression cassette is replaced in the immune cell by a method comprising;
introducing into the immune cell a nucleic acid molecule comprising the expression construct and a 5′ homology arm, wherein the 5′ homology arm is complementary to the 5′ targeting site of the heterologous expression cassette,
such that the expression construct replaces the expression cassette in the genome of the immune cell.

6. A method according to claim 5 wherein the method further comprises introducing into the immune cell a vector comprising a nucleic acid that encodes a CRISPR/Cas9 targeting the targeting site.

7. A method according to any one of the preceding claims wherein the targeting site is a 5′ targeting site.

8. A method according to claim 7 wherein the cassette further comprises a 3′ targeting site.

9. A method according to claim 8 wherein the heterologous expression cassette is replaced in the immune cell by a method comprising;
introducing into the immune cell a nucleic acid molecule, such as a DNA molecule, comprising the expression construct flanked by 5′ and 3′ homology arms, wherein the 5′ and 3′ homology arms are complementary to the 5′ and 3′ targeting sites of the heterologous expression cassette,
such that the expression construct replaces the expression cassette in the genome of the immune cell.

10. A method according to claim 9 wherein the method further comprises introducing into the immune cell a vector comprising a nucleic acid that encodes a CRISPR/Cas9 targeting the 5′ and 3′ targeting sites.

11. A method according to any one of the preceding claims wherein the therapeutic antigen receptor is a T cell receptor (TCR).

12. A method according to claim 11 wherein the therapeutic TCR specifically binds to cancer cells.

13. A method according to any one of the preceding claims wherein the expression cassette comprises 5′ and 3′ targeting sites and said targeting sites comprise a nucleotide sequence from the TCRα chain constant region.

14. A method according to claim 13 wherein the 3′ targeting site is located within the coding sequence for the production TCR.

15. A method according to any one of the preceding claims wherein the immune cell comprising the heterologous expression cassette is produced by a method comprising
(i) transfecting an iPSC with a nucleic acid comprising a heterologous expression cassette, such that the cassette is integrated into the genome of the iPSC,
wherein the expression cassette comprises
(a) a coding sequence for a production T cell receptor (TCR),
(b) a constitutive promoter operably linked to the coding sequence, and
(c) a targeting site, and
(ii) differentiating the iPSC into an immune cell comprising the heterologous expression cassette.

16. A method according to claim 15, wherein the targeting site is a 5′ targeting site.

17. A method according to claim 16 wherein the cassette further comprises a 3′ targeting site.

18. A method according to claim 15, 16 or 17 wherein the iPSCs are differentiated into immune cells by a method that comprises;
(i) differentiating the iPSCs into mesoderm cells,
(ii) differentiating the mesoderm cells into haemogenic endothelial cells,
(iii) differentiating the haemogenic endothelial cells into a population of HPCs,
(iv) differentiating the HPCs into immune cell progenitors; and
(v) maturing the population of progenitor immune cells to produce a population of immune cells.

19. A method according to claim 18 wherein the iPSCs are cultured sequentially in first, second and third mesoderm induction media to induce differentiation into mesoderm cells,
the first mesoderm induction medium consisting of a chemically defined nutrient medium supplemented with one or more differentiation factors, wherein the one or more differentiation factors consist of activin,
the second mesoderm induction medium consisting of a chemically defined nutrient medium supplemented with one or more differentiation factors, wherein the one or more differentiation factors consist of activin, BMP, and FGF, and
the third mesoderm induction medium consisting of a chemically defined nutrient medium supplemented with one or more differentiation factors, wherein the one or more differentiation factors consist of activin, BMP, FGF, and a GSK3 inhibitor.

20. A method according to claim 18 or 19 wherein the mesoderm cells are cultured in an HE induction medium to induce differentiation into HECs; said HE induction medium consisting of a chemically defined nutrient medium supplemented with one or more differentiation factors, wherein the one or more differentiation factors consist of SCF and VEGF.

21. A method according to any one of claims 18 to 20 wherein the HECs are cultured in an haematopoietic induction medium to induce differentiation into HPCs; said haematopoietic induction medium consisting of a chemically defined nutrient medium supplemented with one or more differentiation factors, wherein the one or more differentiation factors consist of VEGF, SCF, Thrombopoietin (TPO), Flt3 ligand (FIt3L), IL-3, IL-6, IL-7, IL-11, IGF-1, BMP, FGF, Sonic hedgehog (SHH), erythropoietin (EPO), angiotensin Il, and an angiotensin II type 1 receptor (AT1) antagonist.

22. A method according to any one of claims 18 to 21 wherein the HPCs are cultured in a lymphoid expansion medium to produce the progenitor immune cells; said lymphoid expansion medium consisting of a chemically defined nutrient medium supplemented with one or more differentiation factors, wherein the one or more differentiation factors consist of SCF, FLT3L, TPO and IL7.

23. A method according to any one of claims 18 to 22 wherein the progenitor immune cells are matured by a method comprising culturing the population of progenitor immune cells in a maturation medium to produce the immune cells; said maturation medium consisting of a chemically defined nutrient medium supplemented with one or more differentiation factors, wherein the one or more differentiation factors consist of SCF, FLT3L, and IL7.

24. A method according to any one of the preceding claims comprising concentrating the population of immune cells expressing the therapeutic antigen receptor.

25. A method according to according to any one of the preceding claims comprising storing the population of immune cells expressing the therapeutic antigen receptor.

26. A method according to any one of the preceding claims comprising formulating the population of immune cells expressing the therapeutic antigen receptor with a pharmaceutically acceptable excipient.

27. A population of immune cells expressing one or more therapeutic antigen receptors produced by a method according to any one of claims 1 to 26.

28. A pharmaceutical composition comprising a population of immune cells expressing one or more therapeutic antigen receptors produced by a method according to any one of claims 1 to 26 and a pharmaceutically acceptable excipient.

29. A population of immune cells expressing one or more therapeutic antigen receptors produced by a method according to any one of claims 1 to 26 for use in a method of treatment.

30. A population of immune cells expressing one or more therapeutic antigen receptors produced by a method according to any one of claims 1 to 26 for use in a method of treatment of cancer.

31. A method of treatment of cancer comprising administering a population of immune cells expressing one or more therapeutic antigen receptors produced by a method according to any one of claims 1 to 26 to an individual in need thereof.

32. Use of a population of immune cells expressing one or more therapeutic antigen receptors produced by a method according to any one of claims 1 to 26 in the manufacture of a medicament for use in treating cancer.

33. An immune cell comprising a heterologous expression cassette integrated into the genome thereof,
wherein the expression cassette comprises
(a) a coding sequence for a production T cell receptor (TCR),
(b) a constitutive promoter operably linked to the coding sequence, and
(c) a targeting site.

34. An immune cell according to claim 31 wherein the targeting site is a 5′ targeting site.

35. An immune cell according to claim 34 wherein the cassette further comprises a 3′ targeting site.

36. An iPSC comprising a heterologous expression cassette integrated into the genome thereof,
wherein the expression cassette comprises
(a) a coding sequence for a production T cell receptor (TCR),
(b) a constitutive promoter operably linked to the coding sequence, and
(c) a targeting site.

37. An iPSC according to claim 33 wherein the targeting site is a 5′ targeting site.

38. An iPSC according to claim 37 wherein the cassette further comprises a 3′ targeting site.


SEQUENCE LISTING (91 sequences)

SEQ ID NO: 1 - protein, 22 aa, synthetic construct
GSGATNFSLLKQAGDVEENPGP

SEQ ID NO: 2 - DNA, 66 bp, synthetic construct
  source 1..66: mol_type = other DNA
ggaagcggagctactaacttcagcctgctgaagcaggctggagacgtggaggagaaccctgggcct

SEQ ID NO: 3 - DNA, 230 bp, synthetic construct
  source 1..230: mol_type = other DNA
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 4 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 5 - RNA, 20 nt, Homo sapiens
  source 1..20: mol_type = other RNA
  ncRNA 1..20: ncRNA_class = guide_RNA
tctctcagctggtacacggc

SEQ ID NO: 6 - RNA, 20 nt, Homo sapiens
  source 1..20: mol_type = other RNA
  ncRNA 1..20: ncRNA_class = guide_RNA
agagtctctcagctggtaca

SEQ ID NO: 7 - RNA, 20 nt, Homo sapiens
  source 1..20: mol_type = other RNA
  ncRNA 1..20: ncRNA_class = guide_RNA
gtcccctccaccccacagtg

SEQ ID NO: 8 - RNA, 20 nt, Homo sapiens
  source 1..20: mol_type = other RNA
  ncRNA 1..20: ncRNA_class = guide_RNA
tcacgtcatccagcagagaa

SEQ ID NO: 9 - RNA, 20 nt, Homo sapiens
  source 1..20: mol_type = other RNA
  ncRNA 1..20: ncRNA_class = guide_RNA
gcaagtccagctttaaatca

SEQ ID NO: 10 - DNA, 122 bp, synthetic construct
  source 1..122: mol_type = other DNA
aacttgtttattgcagcttataatggttacaaataaagcaatagcatcacaaatttcacaaataaagcatttttttcactgcattctagttgtggtttgtccaaactcatcaatgtatctta

SEQ ID NO: 11 - DNA, 24 bp, synthetic construct
  source 1..24: mol_type = other DNA
cgggccaagagaagcggatccggc

SEQ ID NO: 12 - protein, 8 aa, synthetic construct
RAKRSGSG

SEQ ID NO: 13 - DNA, 48 bp, synthetic construct
  source 1..48: mol_type = other DNA
cagaaccctgaccctgccgtgtaccagctgagagactctaaatccagt

SEQ ID NO: 14 - DNA, 924 bp, synthetic construct
  source 1..924: mol_type = other DNA
atgtctctgggcctgctgtgctgtggcgtgttctccctgctgtgggccggacctgtgaatgccggcgtgacccagacccccaagttccgggtgctgaaaaccggccagagcatgacactgctgtgcgcccaggacatgaaccacgactacatgtattggtacagacaggaccccggcatgggcctgcggctgatccactattctgtgggcgagggcaccaccgccaagggcgaagtgcctgatggctacaacgtgtcccggctgaagaagcagaacttcctgctgggcctggaaagcgccgctcctagccagaccagcgtgtacttctgcgccagcagcttcaccgacacccagtacttcggccctggcaccagactgaccgtgctggaggacctgaagaacgtgttccccccagaggtggccgtgttcgagccctctgaggccgagatcagccacacccagaaagccaccctggtctgcctggccaccggcttctaccccgaccacgtggaactgtcttggtgggtgaacggcaaagaggtgcacagcggcgtcagcaccgaccctcagcccctgaaagagcagcccgccctgaacgacagccggtactgcctgagcagcagactgcgggtgtccgccaccttctggcagaacccccggaaccacttcagatgccaggtgcagttctacggcctgagcgagaacgacgagtggacccaggaccgggccaagcctgtgacccagatcgtgtctgccgaagcatgggggcgcgccgattgcggcttcacaagcgagagctaccagcagggcgtgctgagcgccaccatcctgtacgagatcctgctgggcaaggccaccctgtacgccgtgctggtgtccgctctggtgctgatggccatggtgaaacggaaggacagccggggc

SEQ ID NO: 15 - protein, 308 aa, synthetic construct
MSLGLLCCGVFSLLWAGPVNAGVTQTPKFRVLKTGQSMTLLCAQDMNHDYMYWYRQDPGMGLRLIHYSVGEGTTAKGEVPDGYNVSRLKKQNFLLGLESAAPSQTSVYFCASSFTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 16 - DNA, 822 bp, synthetic construct
  source 1..822: mol_type = other DNA
atgatgaagtccctgcgggtgctgctggtcatcctgtggctgcagctgtcctgggtctggtcccagcagaaagaggtggagcagaacagcggccctctgagcgtgcccgagggcgctatcgccagcctgaactgcacctacagcgacagaggcagccagagcttcttctggtacagacagtacagcggcaagagccccgagctgatcatgagcatctacagcaacggcgacaaagaggacggccggttcaccgcccagctgaacaaggccagccagtacgtgtccctgctgatccgggacagccagcccagcgacagcgccacctacctgtgcgccgtgagaggcacaggcagaagggccctgacatttggcagcggcaccagactgcaggtgcagcccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 17 - protein, 274 aa, synthetic construct
MMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVRGTGRRALTFGSGTRLQVQPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 18 - DNA, 1000 bp, Homo sapiens
  source 1..1000: mol_type = other DNA
caaattcacattgcaaagaaatgtggatacaggaaggaaaataagttttatattcttgtaatcgatctatcgtgtataccctctatgtggtagtaactgtagatggtcatctgggaattaatccttattcacagtgtaaacttaattactcactaaaatatataaagcttttaatcatgtatgatattgagatttcatatcttggtacttaaaaatgtatcaaatgcttgctatgtgctcttgctataaagagctaattggtatgagggaaagccaggtatttactaatcaatgtagtgagtaaaatgacagaaaaattataagaagaacatgaatgagggcatttaatttaaactttaggaatcaagaaacgcttctcgaagcagtgattcctgccctgattcttaaataatgtgtaggcattagacaggaggataagtacaaaacgtggcatcatgagcaaaggcatggaaatggcccatgagcggagtgaacactggtttggggttgctccaaggtaaagttcaaaaagtatcctgcagtcaaccctttagcaccataaagaaactaaattatttagatgtttttatgagaacatatcaaaaagtacttttctgtcatccaatacttccacaaataaatcattagttcttgctaatcttcatctggcataaaaataatgacatcaactttcttcatgtaatttcccacttaattcctttactaggagcaatatcaattcctatatgacgtcattgccagcacctaccctgctcagaatggacaagtaaagaaaaacaaccatcaagaagataaaattgaatttgataatgaagtggacaaagtaaagcaggatgctaattgtgttaatccacttggtgccccagaaaagctccctgaagcaaaggaacaggctgaaggttctgaacccacgagtggcactgaggggccagaacattctgtcaatggtcctgctagccctgcattgaaccaaggttca

SEQ ID NO: 19 - DNA, 1000 bp, Homo sapiens
  source 1..1000: mol_type = other DNA
gaaaagacataaatgaggaaactccaaacctcctgttagctgttatttctatttttgtagaagtaggaagtgaaaataggtatacagtggattaattaaatgcagcgaaccaatatttgtagaagggttatattttactactgtggaaaaatatttaagatagttttgccagaacagtttgtacagacgtatgcttattttaaaattttatctcttattcagtaaaaaacaacttctttgtaatcgttatgtgtgtatatgtatgtgtgtatgggtgtgtgtttgtgtgagagacagagaaagagagagaattctttcaagtgaatctaaaagcttttgcttttcctttgtttttatgaagaaaaaatacattttatattagaagtgttaacttagcttgaaggatctgtttttaaaaatcataaactgtgtgcagactcaataaaatcatgtacatttctgaaatgacctcaagatgtcctccttgttctactcatatatatctatcttatatagtttactattttacttctagagatagtacataaaggtggtatgtgtgtgtatgctactacaaaaaagttgttaactaaattaacattgggaaatcttatattccatatattagcatttagtccaatgtctttttaagcttatttaattaaaaaatttccagtgagcttatcatgctgtctttacatggggttttcaattttgcatgctcgattattccctgtacaatatttaaaatttattgcttgatacttttgacaacaaattaggttttgtacaattgaacttaaataaatgtcattaaaataaataaatgcaatatgtattaatattcattgtataaaaatagaagaatacaaacatatttgttaaatatttacatatgaaatttaatatagctatttttatggaatttttcattgatatgaaaaatatgatattgcatatgcatagttcccatgttaaatcccattcataactttcatta

SEQ ID NO: 20 - DNA, 1000 bp, Homo sapiens
  source 1..1000: mol_type = other DNA
gctcccatagctcagtctggtctatctgcctggccctggccattgtcactttgcgctgccctcctctcgcccccgagtgcccttgctgtgccgccggaactctgccctctaacgctgccgtctctctcctgagtccggaccactttgagctctactggcttctgcgccgcctctggcccactgtttccccttcccaggcaggtcctgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccacccca

SEQ ID NO: 21 - DNA, 1001 bp, Homo sapiens
  source 1..1001: mol_type = other DNA
cagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgttcccctgcatcccccttcccctgcatcccccagaggccccaggccacctacttggcctggaccccacgagaggccaccccagccctgtctaccaggctgccttttgggtggattctcctcca

SEQ ID NO: 22 - DNA, 1500 bp, Homo sapiens
  source 1..1500: mol_type = other DNA
ttaattttctttccttcactcctgtatcgatttgtgttgtgtaacaaaccacccccaaatttgggagcctaaacaaataacatttattatggttcagtagtctaatgatatgctggaatgttctgctggtgccctcaggctcaggcaatagagaccaggctgactcatgtgtctgccttcagctgatgtgtgccctacagtttggctggtctaaagtgacctacactgccagtaggctggcatgtggctgccatttagctatggcaacaacagtgagtgggccacatgtccctcctcatccagaagactagcccaggcctattcacattaaagcagcaagttccacaagggaaagaagacttgtgtgagaccacttgaggcccaggcttaaaagtgacacacatgtcttcttctgtatgttattagccaaataaataagtcataaagcctgcccagattcaaggggtagggaaatagactccacttcttgagagggcctgcaaattcacattgcaaagaaatgtggatacaggaaggaaaataagttttatattcttgtaatcgatctatcgtgtataccctctatgtggtagtaactgtagatggtcatctgggaattaatccttattcacagtgtaaacttaattactcactaaaatatataaagcttttaatcatgtatgatattgagatttcatatcttggtacttaaaaatgtatcaaatgcttgctatgtgctcttgctataaagagctaattggtatgagggaaagccaggtatttactaatcaatgtagtgagtaaaatgacagaaaaattataagaagaacatgaatgagggcatttaatttaaactttaggaatcaagaaacgcttctcgaagcagtgattcctgccctgattcttaaataatgtgtaggcattagacaggaggataagtacaaaacgtggcatcatgagcaaaggcatggaaatggcccatgagcggagtgaacactggtttggggttgctccaaggtaaagttcaaaaagtatcctgcagtcaaccctttagcaccataaagaaactaaattatttagatgtttttatgagaacatatcaaaaagtacttttctgtcatccaatacttccacaaataaatcattagttcttgctaatcttcatctggcataaaaataatgacatcaactttcttcatgtaatttcccacttaattcctttactaggagcaatatcaattcctatatgacgtcattgccagcacctaccctgctcagaatggacaagtaaagaaaaacaaccatcaagaagataaaattgaatttgataatgaagtggacaaagtaaagcaggatgctaattgtgttaatccacttggtgccccagaaaagctccctgaagcaaaggaacaggctgaaggttctgaacccacgagtggcactgaggggccagaacattctgtcaatggtcctgctagccctgcattgaaccaaggttca

SEQ ID NO: 23 - DNA, 1621 bp, synthetic construct
  source 1..1621: mol_type = other DNA
cagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaaaagacataaatgaggaaactccaaacctcctgttagctgttatttctatttttgtagaagtaggaagtgaaaataggtatacagtggattaattaaatgcagcgaaccaatatttgtagaagggttatattttactactgtggaaaaatatttaagatagttttgccagaacagtttgtacagacgtatgcttattttaaaattttatctcttattcagtaaaaaacaacttctttgtaatcgttatgtgtgtatatgtatgtgtgtatgggtgtgtgtttgtgtgagagacagagaaagagagagaattctttcaagtgaatctaaaagcttttgcttttcctttgtttttatgaagaaaaaatacattttatattagaagtgttaacttagcttgaaggatctgtttttaaaaatcataaactgtgtgcagactcaataaaatcatgtacatttctgaaatgacctcaagatgtcctccttgttctactcatatatatctatcttatatagtttactattttacttctagagatagtacataaaggtggtatgtgtgtgtatgctactacaaaaaagttgttaactaaattaacattgggaaatcttatattccatatattagcatttagtccaatgtctttttaagcttatttaattaaaaaatttccagtgagcttatcatgctgtctttacatggggttttcaattttgcatgctcgattattccctgtacaatatttaaaatttattgcttgatacttttgacaacaaattaggttttgtacaattgaacttaaataaatgtcattaaaataaataaatgcaatatgtattaatattcattgtataaaaatagaagaatacaaacatatttgttaaatatttacatatgaaatttaatatagctatttttatggaatttttcattgatatgaaaaatatgatattgcatatgcatagttcccatgttaaatcccattcataactttcatta

SEQ ID NO: 24 - DNA, 36 bp, synthetic construct
  source 1..36: mol_type = other DNA
agttcaggttcaagagctaaaaggagcggatcaggt

SEQ ID NO: 25 - DNA, 30 bp, synthetic construct
  source 1..30: mol_type = other DNA
ggcagccgggccaagagatctggatccggc

SEQ ID NO: 26 - protein, 11 aa, synthetic construct
SSGSRAKRSGS

SEQ ID NO: 27 - protein, 10 aa, synthetic construct
GSRAKRSGSG

SEQ ID NO: 28 - DNA, 54 bp, synthetic construct
  source 1..54: mol_type = other DNA
gagggcagaggcagcctgctgacatgtggcgacgtggaagaaaaccctggccct

SEQ ID NO: 29 - protein, 18 aa, synthetic construct
EGRGSLLTCGDVEENPGP

SEQ ID NO: 30 - DNA, 57 bp, synthetic construct
  source 1..57: mol_type = other DNA
gctaccaactttagcctgctgaagcaggccggggacgtggaagaaaaccctggccct

SEQ ID NO: 31 - protein, 19 aa, synthetic construct
ATNFSLLKQAGDVEENPGP

SEQ ID NO: 32 - DNA, 933 bp, synthetic construct
  source 1..933: mol_type = other DNA
atggccagcctgctgttcttctgcggcgccttctacctgctgggcaccggctctatggatgccgacgtgacccagaccccccggaacagaatcaccaagaccggcaagcggatcatgctggaatgctcccagaccaagggccacgaccggatgtactggtacagacaggaccctggcctgggcctgcggctgatctactacagcttcgacgtgaaggacatcaacaagggcgagatcagcgacggctacagcgtgtccagacaggctcaggccaagttcagcctgtccctggaaagcgccatccccaaccagaccgccctgtacttttgtgccacaagcggccagggcgcctacaacgagcagttctttggccctggcacccggctgacagtgctggaagatctgaagaacgtgttccccccagaggtggccgtgttcgagccttctgaggccgaaatcagccacacccagaaagccacactcgtgtgtctggccaccggcttctaccccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaaccccagaaaccacttcagatgccaggtgcagttttacggcctgagcgagaacgacgagtggacccaggacagagccaagcccgtgacacagatcgtgtctgccgaagcttgggggcgcgccgattgtggctttaccagcgagagctaccagcagggcgtgctgagcgccaccatcctgtacgagatcctgctgggaaaggccacactgtacgccgtgctggtgtctgccctggtgctgatggccatggtcaagcggaaggacagccggggc

SEQ ID NO: 33 - DNA, 831 bp, synthetic construct
  source 1..831: mol_type = other DNA
atgaagaagcacctgaccacctttctcgtgatcctgtggctgtacttctaccggggcaacggcaagaaccaggtggaacagagcccccagagcctgatcatcctggaaggcaagaactgcaccctgcagtgcaactacaccgtgtcccccttcagcaacctgcggtggtacaagcaggacaccggcagaggccctgtgtccctgaccatcgtgaccttcagcgagaacaccaagagcaacggccggtacaccgccaccctggacgccgatacaaagcagagcagcctgcacatcaccgccagccagctgagcgatagcgccagctacatctgcgtggtgtccggcggcacagacagctggggcaagctgcagtttggcgccggaacacaggtggtcgtgacccccgacatccagaaccctgaccctgcagtatatcagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 34 - protein, 737 aa, synthetic construct
EQYQFLYDVIASTYPAQNGQVKKNNHQEDKIEFDNEVDKVKQDANCVNPLGAPEKLPEAKEQAEGSEPTSGTEGPEHSVNGPASPALNQGSSSGSRAKRSGSGEGRGSLLTCGDVEENPGPMASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSRAKRSGSGATNFSLLKQAGDVEENPGPMKKHLTTFLVILWLYFYRGNGKNQVEQSPQSLIILEGKNCTLQCNYTVSPFSNLRWYKQDTGRGPVSLTIVTFSENTKSNGRYTATLDADTKQSSLHITASQLSDSASYICVVSGGTDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 35 - DNA, 1500 bp, synthetic construct
  source 1..1500: mol_type = other DNA
cccgcgccgtgctggactccaccaacgccgacggtatcagcgccctgcaccaggtcagcgccccccgcccggcgtctcccggggccaggtccaccctctgctgcgccacctggggcatcctccttccccgttgccagtctcgatccgccccgtcgttcctggccctgggctttgccaccctatgctgacaccccgtcccagtcccccttaccattccccttcgaccaccccacttccgaattggagccgcttcaactggccctgggcttagccactctgtgctgaccactctgccccaggcctccttaccattccccttcgacctactctcttccgcattggagtcgctttaactggccctggctttggcagcctgtgctgacccatgcagtcctccttaccatccctccctcgacttcccctcttccgatgttgagcccctccagccggtcctggactttgtctccttccctgccctgccctctcctgaacctgagccagctcccatagctcagtctggtctatctgcctggccctggccattgtcactttgcgctgccctcctctcgcccccgagtgcccttgctgtgccgccggaactctgccctctaacgctgccgtctctctcctgagtccggaccactttgagctctactggcttctgcgccgcctctggcccactgtttccccttcccaggcaggtcctgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccacccca

SEQ ID NO: 36 - DNA, 230 bp, synthetic construct; identical to SEQ ID NO: 3
  source 1..230: mol_type = other DNA

SEQ ID NO: 37 - DNA, 933 bp, synthetic construct; identical to SEQ ID NO: 32
  source 1..933: mol_type = other DNA

SEQ ID NO: 38 - DNA, 831 bp, synthetic construct; identical to SEQ ID NO: 33
  source 1..831: mol_type = other DNA

SEQ ID NO: 39 - protein, 616 aa, synthetic construct
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSRAKRSGSGATNFSLLKQAGDVEENPGPMKKHLTTFLVILWLYFYRGNGKNQVEQSPQSLIILEGKNCTLQCNYTVSPFSNLRWYKQDTGRGPVSLTIVTFSENTKSNGRYTATLDADTKQSSLHITASQLSDSASYICVVSGGTDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 40 - DNA, 225 bp, synthetic construct; identical to SEQ ID NO: 4
  source 1..225: mol_type = other DNA

SEQ ID NO: 41 - DNA, 1622 bp, synthetic construct
  source 1..1622: mol_type = other DNA
cagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgttcccctgcatcccccttcccctgcatcccccagaggccccaggccacctacttggcctggaccccacgagaggccaccccagccctgtctaccaggctgccttttgggtggattctcctcca

SEQ ID NO: 42 - DNA, 58 bp, synthetic construct
  source 1..58: mol_type = other DNA
ttcaggtttactcacgtcatccagcagagaatggaaagtcaggctccggtgcccgtca

SEQ ID NO: 43 - DNA, 67 bp, synthetic construct
  source 1..67: mol_type = other DNA
tgactttccattctctgctggatgacgtgagtaaacctgaaaacttgtttattgcagcttataatgg

SEQ ID NO: 44 - DNA, 100 bp, synthetic construct
  source 1..100: mol_type = other DNA
cgacctttggtcgcccggcctcagtgagcgagcgcgcagagagggagtggccaactccatcactaggggttcctgcggccgctgggttcccttttccttc

SEQ ID NO: 45 - DNA, 59 bp, synthetic construct
  source 1..59: mol_type = other DNA
gactttccattctctgctggatgacgtgagtaaacctgaatgtggggtggaggggacag

SEQ ID NO: 46 - DNA, 64 bp, synthetic construct
  source 1..64: mol_type = other DNA
tcaggtttactcacgtcatccagcagagaatggaaagtcagtggggccactagggacaggattg

SEQ ID NO: 47 - DNA, 61 bp, synthetic construct
  source 1..61: mol_type = other DNA
gagggagtggccaactccatcactaggggttcctgcggccgctactggccttatctcacag

SEQ ID NO: 48 - DNA, 93 bp, synthetic construct
  source 1..93: mol_type = other DNA
gtcgatcctaccatccactcgacacacccgccagcggccgctgccaagcttgccgccaccatgaagaagcacctgaccacctttctcgtgatc

SEQ ID NO: 49 - DNA, 62 bp, synthetic construct
  source 1..62: mol_type = other DNA
ccaatcctgtccctagtggccccactgactttccattcccatagagcccaccgcatccccag

SEQ ID NO: 50 - DNA, 100 bp, synthetic construct; identical to SEQ ID NO: 44
  source 1..100: mol_type = other DNA

SEQ ID NO: 51 - DNA, 88 bp, synthetic construct
  source 1..88: mol_type = other DNA
gtgggcgatgtgcgctctgcccactgacgggcaccggagcctctgctggatgacgtgagtaaacctgaatgtggggtggaggggacag

SEQ ID NO: 52 - DNA, 38 bp, synthetic construct
  source 1..38: mol_type = other DNA
gaatggaaagtcagtggggccactagggacaggattgg

SEQ ID NO: 53 - DNA, 100 bp, synthetic construct
  source 1..100: mol_type = other DNA
tttggtcgcccggcctcagtgagcgagcgagcgcgcagagagggagtggccaactccatcactaggggttcctgcggccgctactggccttatctcacag

SEQ ID NO: 54 - DNA, 20 bp, synthetic construct
  source 1..20: mol_type = other DNA
ggatgctctttccggagcac

SEQ ID NO: 55 - DNA, 20 bp, synthetic construct
  source 1..20: mol_type = other DNA
gcaccggttcaattgccgac

SEQ ID NO: 56 - DNA, 20 bp, synthetic construct
  source 1..20: mol_type = other DNA
tggtgaacacctaggacgca

SEQ ID NO: 57 - DNA, 19 bp, synthetic construct
  source 1..19: mol_type = other DNA
ggctctcggagaatgacga

SEQ ID NO: 58 - DNA, 3282 bp, synthetic construct
  source 1..3282: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcactagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaaaagatctttgtcgatcctaccatccactcgacacacccgccagcggccgctgccaagcttgccgccaccatgatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgagaggcacgggcaggagagcacttacttttgggagtggaacaagactccaagtgcaaccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggcagccgggccaagagaagcggatccggcgccaccaacttcagcctgctgaagcaggccggcgacgtggaggaaaaccctggccctaggatgagcctcgggctcctgtgctgtggggtgttttctctcctgtgggcaggtccagtgaatgctggtgtcactcagaccccaaaattccgggtcctgaagacaggacagagcatgacactgctgtgtgcccaggatatgaaccatgaatacatgtactggtatcgacaagacccaggcatggggctgaggctgattcattactcagttgccgagggtacaactgccaaaggagaggtccctgatggctacaatgtctccagattaaaaaaacagaatttcctgctggggttggagtcggctgctccctcccaaacatctgtgtacttctgtgccagcagtttcacagatacgcagtattttggcccaggcacccggctgacagtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctaataaggctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 59 - DNA, 7242 bp, synthetic construct
  source 1..7242: mol_type = other DNA
cctgcaggcagctgcgcgctcgctcgctcactgaggccgcccgggcgtcgggcgacctttggtcgcccggcctcagtgagcgagcgcgcagagagggagtggccaactccatcactaggggttcctgcggccactgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacattcaggtttactcacgtcatccagcagagaatggaaagtcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcactagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaaaagatctttgtcgatcctaccatccactcgacacacccgccagcggccgctgccaagcttgccgccaccatgatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgagaggcacgggcaggagagcacttacttttgggagtggaacaagactccaagtgcaaccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggcagccgggccaagagaagcggatccggcgccaccaacttcagcctgctgaagcaggccggcgacgtggaggaaaaccctggccctaggatgagcctcgggctcctgtgctgtggggtgttttctctcctgtgggcaggtccagtgaatgctggtgtcactcagaccccaaaattccgggtcctgaagacaggacagagcatgacactgctgtgtgcccaggatatgaaccatgaatacatgtactggtatcgacaagacccaggcatggggctgaggctgattcattactcagttgccgagggtacaactgccaaaggagaggtccctgatggctacaatgtctccagattaaaaaaacagaatttcctgctggggttggagtcggctgctccctcccaaacatctgtgtacttctgtgccagcagtttcacagatacgcagtattttggcccaggcacccggctgacagtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctaataaggctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttttcaggtttactcacgtcatccagcagagaatggaaagtcagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 60 - protein, 612 aa, synthetic construct
MMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVRGTGRRALTFGSGTRLQVQPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSRAKRSGSGATNFSLLKQAGDVEENPGPRMSLGLLCCGVFSLLWAGPVNAGVTQTPKFRVLKTGQSMTLLCAQDMNHEYMYWYRQDPGMGLRLIHYSVAEGTTAKGEVPDGYNVSRLKKQNFLLGLESAAPSQTSVYFCASSFTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 61 - DNA, 7266 bp, synthetic construct
  source 1..7266: mol_type = other DNA
gctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacattcaggtttactcacgtcatccagcagaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcactagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaaaagatctttgtcgatcctaccatccactcgacacacccgccagcggccgctgccaagcttgccgccaccatgaagaagcacctgaccacctttctcgtgatcctgtggctgtacttctaccggggcaacggcaagaaccaggtggaacagagcccccagagcctgatcatcctggaaggcaagaactgcaccctgcagtgcaactacaccgtgtcccccttcagcaacctgcggtggtacaagcaggacaccggcagaggccctgtgtccctgaccatcctgaccttcagcgagaacaccaagagcaacggccggtacaccgccaccctggacgccgatacaaagcagagcagcctgcacatcaccgccagccagctgagcgatagcgccagctacatctgcgtggtgtccggcggcacagacagctggggcaagctgcagtttggcgccggaacacaggtggtcgtgacccccgacatccagaaccctgaccctgccgtgtaccagctgcgggacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaatagcgccgtggcctggtccaacaagagcgacttcgcctgcgccaacgccttcaacaacagcattatccccgaggacacattcttcccaagccccgagagcagctgcgacgtcaagctggtggaaaagagcttcgagacagacaccaacctgaacttccagaacctgagcgtgatcggcttcagaatcctgctgctgaaggtggccggcttcaacctgctgatgaccctgagactgtggtccagcggcagccgggccaagagatctggatccggcgctaccaactttagcctgctgaagcaggccggggacgtggaagaaaaccctggccctaggatggccagcctgctgttcttctgcggcgccttctacctgctgggcaccggctctatggatgccgacgtgacccagaccccccggaacagaatcaccaagaccggcaagcggatcatgctggaatgctcccagaccaagggccacgaccggatgtactggtacagacaggaccctggcctgggcctgcggctgatctactacagcttcgacgtgaaggacatcaacaagggcgagatcagcgacggctacagcgtgtccagacaggctcaggccaagttcagcctgtccctggaaagcgccatccccaaccagaccgccctgtacttttgtgccacaagcggccagggcgcctacgaggagcagttctttggccctggcacccggctgacagtgctggaagatctgaagaacgtgttccccccagaggtggccgtgttcgagccttctgaggccgaaatcagccacacccagaaagccacactcgtgtgtctggccaccggcttctaccccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaaccccagaaaccacttcagatgccaggtgcagttttacggcctgagcgagaacgacgagtggacccaggacagagccaagcccgtgacacagatcgtgtctgccgaagcttgggggcgcgccgattgtggctttaccagcgagagctaccagcagggcgtgctgagcgccaccatcctgtacgagatcctgctgggaaaggccacactgtacgccgtgctggtgtctgccctggtgctgatggccatggtcaagcggaaggacagccggggctaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaatggaaagtcagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcgtcgggcgacctttggtcgcccggcctcagtgagcgagcgcgcagagagggagtggccaactccatcactaggggttcctgcggcc

SEQ ID NO: 62 - protein, 617 aa, synthetic construct
MKKHLTTFLVILWLYFYRGNGKNQVEQSPQSLIILEGKNCTLQCNYTVSPFSNLRWYKQDTGRGPVSLTILTFSENTKSNGRYTATLDADTKQSSLHITASQLSDSASYICVVSGGTDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSRAKRSGSGATNFSLLKQAGDVEENPGPRMASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYEEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 63 - RNA, 20 nt, synthetic construct
  source 1..20: mol_type = other RNA
  ncRNA 1..20: ncRNA_class = guide_RNA
tcacgtcatccagcagagaa

SEQ ID NO: 64 - DNA, 499 bp, Homo sapiens
  source 1..499: mol_type = other DNA
ctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccaca

SEQ ID NO: 65 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcactagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 66 - DNA, 822 bp, synthetic construct
  source 1..822: mol_type = other DNA
atgatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgagaggcacgggcaggagagcacttacttttgggagtggaacaagactccaagtgcaaccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 67 - protein, 274 aa, synthetic construct; identical to SEQ ID NO: 17

SEQ ID NO: 68 - DNA, 30 bp, synthetic construct
  source 1..30: mol_type = other DNA
ggcagccgggccaagagaagcggatccggc

SEQ ID NO: 69 - DNA, 60 bp, synthetic construct
  source 1..60: mol_type = other DNA
gccaccaacttcagcctgctgaagcaggccggcgacgtggaggaaaaccctggccctagg

SEQ ID NO: 70 - protein, 20 aa, synthetic construct
ATNFSLLKQAGDVEENPGPR

SEQ ID NO: 71 - DNA, 924 bp, synthetic construct
  source 1..924: mol_type = other DNA
atgagcctcgggctcctgtgctgtggggtgttttctctcctgtgggcaggtccagtgaatgctggtgtcactcagaccccaaaattccgggtcctgaagacaggacagagcatgacactgctgtgtgcccaggatatgaaccatgaatacatgtactggtatcgacaagacccaggcatggggctgaggctgattcattactcagttgccgagggtacaactgccaaaggagaggtccctgatggctacaatgtctccagattaaaaaaacagaatttcctgctggggttggagtcggctgctccctcccaaacatctgtgtacttctgtgccagcagtttcacagatacgcagtattttggcccaggcacccggctgacagtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 72 - protein, 308 aa, synthetic construct
MSLGLLCCGVFSLLWAGPVNAGVTQTPKFRVLKTGQSMTLLCAQDMNHEYMYWYRQDPGMGLRLIHYSVAEGTTAKGEVPDGYNVSRLKKQNFLLGLESAAPSQTSVYFCASSFTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 73 - DNA, 122 bp, synthetic construct
  source 1..122: mol_type = other DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 74 - DNA, 500 bp, Homo sapiens
  source 1..500: mol_type = other DNA
gtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagta

SEQ ID NO: 75 - DNA, 59 bp, synthetic construct; identical to SEQ ID NO: 45
  source 1..59: mol_type = other DNA

SEQ ID NO: 76 - DNA, 3782 bp, synthetic construct
  source 1..3782: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcactagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgactgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacacctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaaaagatctttgtcgatcctaccatccactcgacacacccgccagcggccgctgccaagcttgccgccaccatgatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgagaggcacgggcaggagagcacttacttttgggagtggaacaagactccaagtgcaaccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggcagccgggccaagagaagcggatccggcgccaccaacttcagcctgctgaagcaggccggcgacgtggaggaaaaccctggccctaggatgagcctcgggctcctgtgctgtggggtgttttctctcctgtgggcaggtccagtgaatgctggtgtcactcagaccccaaaattccgggtcctgaagacaggacagagcatgacactgctgtgtgcccaggatatgaaccatgaatacatgtactggtatcgacaagacccaggcatggggctgaggctgattcattactcagttgccgagggtacaactgccaaaggagaggtccctgatggctacaatgtctccagattaaaaaaacagaatttcctgctggggttggagtcggctgctccctcccaaacatctgtgtacttctgtgccagcagtttcacagatacgcagtattttggcccaggcacccggctgacagtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctaataaggctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 77 - DNA, 528 bp, Homo sapiens
  source 1..528: mol_type = other DNA
gctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacattcaggtttactcacgtcatccagcaga

SEQ ID NO: 78 - DNA, 822 bp, synthetic construct
  source 1..822: mol_type = other DNA
atgaagaagcacctgaccacctttctcgtgatcctgtggctgtacttctaccggggcaacggcaagaaccaggtggaacagagcccccagagcctgatcatcctggaaggcaagaactgcaccctgcagtgcaactacaccgtgtcccccttcagcaacctgcggtggtacaagcaggacaccggcagaggccctgtgtccctgaccatcctgaccttcagcgagaacaccaagagcaacggccggtacaccgccaccctggacgccgatacaaagcagagcagcctgcacatcaccgccagccagctgagcgatagcgccagctacatctgcgtggtgtccggcggcacagacagctggggcaagctgcagtttggcgccggaacacaggtggtcgtgacccccgacatccagaaccctgaccctgccgtgtaccagctgcgggacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaatagcgccgtggcctggtccaacaagagcgacttcgcctgcgccaacgccttcaacaacagcattatccccgaggacacattcttcccaagccccgagagcagctgcgacgtcaagctggtggaaaagagcttcgagacagacaccaacctgaacttccagaacctgagcgtgatcggcttcagaatcctgctgctgaaggtggccggcttcaacctgctgatgaccctgagactgtgg

SEQ ID NO: 79 - protein, 274 aa, synthetic construct
MKKHLTTFLVILWLYFYRGNGKNQVEQSPQSLIILEGKNCTLQCNYTVSPFSNLRWYKQDTGRGPVSLTILTFSENTKSNGRYTATLDADTKQSSLHITASQLSDSASYICVVSGGTDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLW

SEQ ID NO: 80 - DNA, 60 bp, synthetic construct
  source 1..60: mol_type = other DNA
gctaccaactttagcctgctgaagcaggccggggacgtggaagaaaaccctggccctagg

SEQ ID NO: 81 - DNA, 936 bp, synthetic construct
  source 1..936: mol_type = other DNA
atggccagcctgctgttcttctgcggcgccttctacctgctgggcaccggctctatggatgccgacgtgacccagaccccccggaacagaatcaccaagaccggcaagcggatcatgctggaatgctcccagaccaagggccacgaccggatgtactggtacagacaggaccctggcctgggcctgcggctgatctactacagcttcgacgtgaaggacatcaacaagggcgagatcagcgacggctacagcgtgtccagacaggctcaggccaagttcagcctgtccctggaaagcgccatccccaaccagaccgccctgtacttttgtgccacaagcggccagggcgcctacgaggagcagttctttggccctggcacccggctgacagtgctggaagatctgaagaacgtgttccccccagaggtggccgtgttcgagccttctgaggccgaaatcagccacacccagaaagccacactcgtgtgtctggccaccggcttctaccccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaaccccagaaaccacttcagatgccaggtgcagttttacggcctgagcgagaacgacgagtggacccaggacagagccaagcccgtgacacagatcgtgtctgccgaagcttgggggcgcgccgattgtggctttaccagcgagagctaccagcagggcgtgctgagcgccaccatcctgtacgagatcctgctgggaaaggccacactgtacgccgtgctggtgtctgccctggtgctgatggccatggtcaagcggaaggacagccggggctaa

SEQ ID NO: 82 - protein, 311 aa, synthetic construct
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYEEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 83 - DNA, 513 bp, Homo sapiens
  source 1..513: mol_type = other DNA
gaatggaaagtcagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagta

SEQ ID NO: 84 - DNA, 23 bp, synthetic construct
  source 1..23: mol_type = other DNA
ggctccggtgcccgtcagtgggc

SEQ ID NO: 85 - DNA, 24 bp, synthetic construct
  source 1..24: mol_type = other DNA
ggtggcggcaagcttggcagcggc

SEQ ID NO: 86 - protein, 10 aa, synthetic construct
GVYDGREHTV

SEQ ID NO: 87 - DNA, 20 bp, synthetic construct
  source 1..20: mol_type = other DNA
tgtaccagctgagagactct

SEQ ID NO: 88 - DNA, 41 bp, synthetic construct
  source 1..41: mol_type = other DNA
ttcaggtttactcacgtcatccagcagagaatggaaagtca

SEQ ID NO: 89 - DNA, 23 bp, synthetic construct
  source 1..23: mol_type = other DNA
cctgccgtgtaccagctgagaga

SEQ ID NO: 90 - DNA, 110 bp, synthetic construct
  source 1..110: mol_type = other DNA
accttatattcccagggccggttaatgtggctctggttctgggtacttttatctgtcccctccaccccacagtggggccactagggacaggattggtgacagaaaagccc

SEQ ID NO: 91 - DNA, 106 bp, synthetic construct
  source 1..106: mol_type = other DNA
ggctctggttctgggtacttttatctgtcccctccaccccacattcaggtttactcacgtcatccagcagagaatggaaagtcaggctccggtgcccgtcagtggg